(19) United States
(12) Patent Application Publication
(10) Pub. No.: US 2024/0401129 A1
(43) Pub. Date: Dec. 5, 2024

(54) METHODS FOR DOUBLE-STRANDED SEQUENCING BY SYNTHESIS

(71) Applicant: ILLUMINA, INC., San Diego, CA (US)

(72) Inventors: Philip Balding, Cambridge (GB); Raphaëlle Hours, Northstowe (GB); Esther Musgrave-Brown, Cambridge (GB); Ida Ertmanska, Cambridge (GB); Niall Gormley, Cambridge (GB); Yuan Heng Trevor Ho, Cambridge (GB)

(73) Assignee: ILLUMINA, INC., San Diego, CA (US)

(21) Appl. No.: 18/678,592

(22) Filed: May 30, 2024

Related U.S. Application Data
(60) Provisional application No. 63/470,050, filed on May 31, 2023.

(51) Int. Cl. C12Q 1/6874

(52) U.S. Cl. CPC C12Q 1/6874

(57) ABSTRACT

Nucleotide sequencing methods for sequencing an oligonucleotide strand while the oligonucleotide strand is a part of a double-stranded complex. At least one strand of the double-stranded complex is immobilized on a surface. Sequencing primers may not be immobilized on a surface. Double stranded sequencing methods may employ an enzyme that has nick-translation activity.

### CROSS REFERENCE TO RELATED APPLICATIONS

This application claims the benefit of U.S. Provisional Patent Application No. 63/470,050, filed May 31, 2023, which is incorporated herein by reference in its entirety.

### SEQUENCE LISTING

This application contains a Sequence Listing electronically submitted to the United States Patent and Trademark Office via Patent Center as an XML file entitled “0531 002596US01” having a size of 10.3 kilobytes and created on May 28, 2024. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### FIELD

The present disclosure relates to, among other things, double-stranded sequencing method of oligonucleotides.

### INTRODUCTION

Many sequencing methods, such as sequencing by synthesis methods, use a single-stranded oligonucleotide as a template. Typically, such single-stranded oligonucleotides may be rapidly and accurately sequenced. However, single-stranded oligonucleotides having sequences that may self-hybridize to form secondary structures present unique oligonucleotide sequencing challenges. For example, single-stranded oligonucleotides that form secondary structures such as G-quadruplexes, stem loops, hairpins, or other self-hybridizing structures are difficult to sequence. The formation of the secondary structure in a single stranded oligonucleotide can result in sequencing errors and/or the inability to sequence that portion of the polynucleotide. As such, it would be desirable to develop sequencing methodologies that allow for the accurate and robust sequencing of oligonucleotides that are capable of forming secondary structures.

### SUMMARY

Presented herein, among other things, are methods and compositions for sequencing at least a portion of a strand of a double-stranded oligonucleotide complex. The presence of a second strand in a double-stranded oligonucleotide complex can prevent the other strand from self-hybridizing and forming secondary structures that can present challenges to sequencing. However, the presence of the second strand in oligonucleotide complex can also result in steric, or other, hinderances to sequencing. Strategies for sequencing double stranded oligonucleotide complexes are described herein.

In one aspect, the present disclosure describes a first method for sequencing a polynucleotide template. The method includes (a) providing a surface-bound double-stranded oligonucleotide complex. The double-stranded oligonucleotide complex includes a first oligonucleotide strand, a second oligonucleotide strand hybridized to the first oligonucleotide strand, and a primer hybridized to the first oligonucleotide strand. The first oligonucleotide strand has a 5′ end bound to the surface. The primer has a free 3′ end that is hybridized to a nucleotide of the first oligonucleotide strand that is 3′ of a nucleotide of the first oligonucleotide strand to which a 5′ end of the second oligonucleotide strand is hybridized. The method further includes (b) extending the primer from the free 3′ end using the first oligonucleotide strand as a template and sequencing at least a portion of the first oligonucleotide strand via sequencing by synthesis as the primer is extended. The method further includes (c) nicking the second strand to remove a 5′ portion of the second strand before, during, or after one or more nucleotides are added to extend the primer in step (b).

In another aspect, the present disclosure describes a second method for sequencing a polynucleotide template. The method includes (a) providing a first surface-bound double-stranded oligonucleotide complex. The double-stranded oligonucleotide complex includes a first oligonucleotide strand and a second oligonucleotide strand. The first oligonucleotide strand has a 5′ end bound to a surface. The method further includes (b) exposing the first surface-bound double-stranded oligonucleotide complex to a nuclease to cleave the second oligonucleotide strand and produce a cleaved first portion and a cleaved second portion of the second oligonucleotide strand. The cleaved first and second portions are hybridized to the first oligonucleotide strand and the cleaved first portion comprises a free 3′ end. The method further includes (c) extending the cleaved first portion from the free 3′ end using the first oligonucleotide strand as a template and sequencing at least a portion of the first oligonucleotide strand as the cleaved first portion is extended. In some embodiments, the second oligonucleotide strand has a 5′ end bound to the surface. In some embodiments, the cleaved first portion of the second oligonucleotide strand has a 5′ end bound to the surface. In some such embodiments, extending the surface bound cleaved first portion generates a second surface-bound double-stranded oligonucleotide complex comprising the first oligonucleotide strand and a nascent third oligonucleotide strand comprising the surface bound first portion.

In another aspect, the present disclosure describes a third method for sequencing a polynucleotide template. The method includes (a) providing a first oligonucleotide strand having a 5′ end bound to a surface. The method further includes (b) hybridizing an extension primer to a portion of the first oligonucleotide strand, the primer having a free 3′ end, a free 5′ end, and a cleavage site. The method further includes (c) extending the extension primer from the free 3′ end using the first oligonucleotide strand as a template to produce a second oligonucleotide strand hybridized to the surface-bound first oligonucleotide strand, the second oligonucleotide strand comprising the extension primer. The method further includes (d) cleaving the extension primer of the second oligonucleotide strand at the cleavage site to produce cleaved first and second portions of the second oligonucleotide strand. The cleaved first and second portions are hybridized to the first oligonucleotide strand and the cleaved first portion has a free 3′ end. The method further includes (e) extending the cleaved first portion from the free 3′ end using the first oligonucleotide strand as a template and sequencing at least a portion of the first oligonucleotide strand as the cleaved first portion is extended. In some embodiments, the first oligonucleotide strand has a 3′ portion, and the method further includes providing a surface oligonucleotide having a free 3′ end and having a 5′ end bound to the surface, the 3′ portion of the first oligonucleotide hybridized to at least a portion of the surface oligonucleotide.

In another aspect, the present disclosure describes a fourth method for sequencing a polynucleotide template. The method includes (a) providing a first oligonucleotide strand having a 5′ end bound to a surface. The method further includes (b) hybridizing an extension primer to a portion of the first oligonucleotide strand, the extension primer having two or more cleavage sites and comprising a free 3′ end. The method further includes (c) extending the extension primer from the free 3′ end using the first oligonucleotide strand as a template to produce a second oligonucleotide strand hybridized to the surface-bound first strand, the second oligonucleotide strand including the extension primer and an extended second portion. The method further includes (d) cleaving the extension primer of the second oligonucleotide at the two or more cleavage sites to produce multiple fragments and a second portion of the second oligonucleotide strand. The second portion of the second oligonucleotide strand includes the extended second portion and the second portion is hybridized to the first oligonucleotide strand. The method further includes (e) hybridizing a sequencing primer to the first oligonucleotide strand. At least a portion of the sequencing primer hybridizes to a region of the first oligonucleotide strand to which at least a portion of the first primer hybridized. The sequencing primer has a free 3′ end. The method further includes (f) extending the sequencing primer from the free 3′ end using the first oligonucleotide strand as a template and sequencing at least a portion of the first oligonucleotide strand as the sequencing primer is extended. In some embodiments, the first oligonucleotide strand has a 3′ portion and the method further includes providing a surface oligonucleotide strand having a free 3′ end and a 5′ end bound to the surface where the 3′ portion of the first oligonucleotide strand is hybridized to at least a portion of the surface oligonucleotide.

The details of one or more embodiments are set forth in the accompanying drawings and the description below. Other features, objects, and advantages will be apparent from the description and drawings, and from the claims.

It is to be understood that both the foregoing general description and the following detailed description present embodiments of the subject matter of the present disclosure and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### BRIEF DESCRIPTION OF DRAWINGS

The following detailed description of specific embodiments of the present disclosure may be best understood when read in conjunction with the following drawings.

FIG. 1 is a flow diagram illustrating an overview of double-stranded sequencing method using nick translation consistent with some embodiments of the present disclosure.

FIG. 2 is a flow diagram illustrating an overview of a first double-stranded sequencing method consistent with some embodiments of the present disclosure.

FIG. 3 is a flow diagram illustrating an overview of a second double-stranded sequencing method consistent with some embodiments of the present disclosure.

FIG. 4 is a flow diagram illustrating an overview of a third double-stranded sequencing method consistent with some embodiments of the present disclosure.

FIG. 5A is a schematic drawing illustrating strand displacement and nick translation.

FIG. 5B is a schematic drawing illustrating a sequencing by synthesis technique consistent with some embodiments of the present disclosure.

FIGS. 6A, 6B, and 6C are schematic drawings illustrating a first sequencing workflow consistent with some embodiments of the present disclosure.

FIGS. 7A and 7B are schematic drawings illustrating a second sequencing workflow consistent with some embodiments of the present disclosure.

FIGS. 8A and 8B are schematic drawings illustrating a third sequencing workflow consistent with some embodiments of the present disclosure.

FIGS. 9A and 9B are example synthetic schemes showing a cleavage reaction at the allyl-T of an oligonucleotide strand using Pd(0) (A) and OsO₄ (B).

FIG. 10 is a first plot showing sequencing performance at a G quadruplex region using sequencing methods consistent with the present disclosure.

FIG. 11 is a second plot showing sequencing performance at a G quadruplex region using sequencing methods consistent with the present disclosure.

FIG. 12 is a schematic diagram illustrating the predicted cleave site of the CRISPR/Cas9 complex used in Example 1.

FIG. 13 is an image of a polyacrylamide gel after a DNA template was sequenced via nick translation using various flap nuclease constructs.

FIG. 14 are plots of sequencing metrics after a DNA template was sequenced via nick translation using various flap nuclease constructs.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### Definitions

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. Thus, for example, reference to a “oligonucleotide strand” includes examples having two or more such “oligonucleotide strands” unless the context clearly indicates otherwise.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

As used herein, “have”, “has”, “having”, “include”, “includes”, “including”, “comprise”, “comprises”, “comprising” or the like are used in their open-ended inclusive sense, and generally mean “include, but not limited to”, “includes, but not limited to”, or “including, but not limited to”.

“Optional” or “optionally” means that the subsequently described event, circumstance, or component, can or cannot occur, and that the description includes instances where the event, circumstance, or component, occurs and instances where it does not.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising an incorporation step, a detection step, a deprotection step, and one or more wash steps includes embodiments where the method consists of enumerated steps and embodiments where the method consists essentially of the enumerated.

As used herein, “providing” in the context of a compound, composition, or article means making the compound, composition, or article; purchasing the compound, composition or article; or otherwise obtaining the compound, composition or article.

As used herein, the term “chain extending enzyme” is an enzyme that produces a copy replicate of a polynucleotide using the polynucleotide as a template strand. For example, the chain extending enzyme may be an enzyme having polymerase activity. Typically, DNA polymerases bind to the template strand and then move down the template strand sequentially adding nucleotides to the free hydroxyl group at the 3′ end of a growing strand of nucleic acid. DNA polymerases typically synthesize complementary DNA molecules from DNA templates and RNA polymerases typically synthesize RNA molecules from DNA templates (transcription). The polymerase may be linked to another protein or domain of a protein such as, for example, a flap nuclease. Polymerases may use a short RNA or DNA strand, called a primer, to begin strand growth. Some polymerases may displace the strand upstream of the site where they are adding bases to a chain. Such polymerases are said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity. Any suitable polymerase may be used with the methods and/or compositions (e.g., kits) of the present disclosure. In some embodiments, the polymerase is a polymerase described in U.S. Provisional Patent Application No. 63/412,241, US Patent Application Number U.S. Ser. No. 16/703,569 (U.S. Ser. No. 11/001,816B2), PCT Application Number PCT/US2013/03169 (WO2014142921A1) all of which are hereby incorporated by reference in its entirety.

The terms “polynucleotide” and “oligonucleotide” and “oligonucleotide strand” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). As used herein, “amplified target sequences” and its derivatives, refers generally to a polynucleotide sequence produced by the amplifying the target sequences using target-specific primers and the methods provided herein. The amplified target sequences may be either of the same sense (e.g., the positive strand) or antisense (i.e., the negative strand) with respect to the target sequences.

The term “polynucleotide template” or “template polynucleotide” refer to a polymeric form of a nucleotide that includes a target nucleic acid and an adaptor on one or both ends.

Suitable nucleotides for use in the provided methods include, but are not limited to, deoxynucleotide triphosphates, deoxyadenosine triphosphate (dATP), deoxythymidine triphosphate (dTTP), deoxycytidine triphosphate (dCTP), and deoxyguanosine triphosphate (dGTP). Optionally, the nucleotides used in the provided methods, whether labeled or unlabeled, can include a blocking moiety such as a reversible terminator moiety that inhibits chain extension. Suitable labels for use on the labeled nucleotides include, but are not limited to, haptens, radionucleotides, enzymes, fluorescent labels, chemiluminescent labels, and chromogenic agents.

A polynucleotide will generally contain phosphodiester bonds, although in some cases nucleic acid analogs can have alternate backbones, comprising, for example, phosphoramidite (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Polynucleotides containing one or more carbocyclic sugars are also included within the definition of polynucleotides (see Jenkins et al., Chem. Soc. Rev. (1995) pg. 169-176). Several polynucleotide analogs are described in Rawls, C & E News Jun. 2, 1997 page 35. All these references are hereby expressly incorporated by reference. These modifications of the ribose-phosphate backbone may be done to facilitate the addition of labels, or to increase the stability and half-life of such molecules in physiological environments.

A polynucleotide will generally contain a specific sequence of four nucleotide bases: adenine (A); cytosine (C); guanine (G); and thymine (T). Uracil (U) can also be present, for example, as a natural replacement for thymine when the nucleic acid is RNA. Uracil can also be used in DNA (dU). A polynucleotide may also include native or non-native bases. In this regard, a native deoxyribonucleic acid polynucleotide may have one or more bases selected from the group consisting of adenine, thymine, cytosine, or guanine and a ribonucleic acid may have one or more bases selected from the group consisting of uracil, adenine, cytosine, or guanine. It will be understood that a deoxyribonucleic acid polynucleotide used in the methods or compositions set forth herein may include, for example, uracil bases and a ribonucleic acid can include, for example, a thymine base. Exemplary non-native bases that may be included in a nucleic acid, whether having a native backbone or analog structure, include, without limitation, inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like.

Optionally, isocytosine and isoguanine may be included in a nucleic acid in order to reduce non-specific hybridization, as generally described in U.S. Pat. No. 5,681,702, which is incorporated by reference herein in its entirety.

A non-native base used in a polynucleotide may have universal base pairing activity such that it is capable of base pairing with any other naturally occurring base. Exemplary bases having universal base pairing activity include 3-nitropyrrole and 5-nitroindole. Other bases that can be used include those that have base pairing activity with a subset of the naturally occurring bases such as inosine, which base pairs with cytosine, adenine or uracil.

Incorporation of a nucleotide into a polynucleotide strand refers to joining of the nucleotide to a free 3′ hydroxyl group of the polynucleotide strand via formation of a phosphodiester linkage with the 5′ phosphate group of the nucleotide. The polynucleotide template to be sequenced can be DNA or RNA, or even a hybrid molecule that includes both deoxynucleotides and ribonucleotides. The polynucleotide can include naturally occurring and/or non-naturally occurring nucleotides and natural or non-natural backbone linkages.

The terms “primer oligonucleotide”, “oligonucleotide primer”, and “primer” are used throughout interchangeably and are polynucleotide sequences that are capable of annealing specifically to one or more polynucleotide templates to be amplified or sequenced. Generally, primer oligonucleotides are single-stranded or partially single-stranded. Primers may also contain a mixture of non-natural bases, non-nucleotide chemical modifications or non-natural backbone linkages so long as the non-natural entities do not interfere with the function of the primer. Typically, the primer functions as a substrate onto which nucleotides may be polymerized by a polymerase; in some embodiments, however, the primer may become incorporated into the synthesized polynucleotide strand and provide a site to which another primer may hybridize to prime synthesis of a new strand that is complementary to the synthesized nucleic acid molecule. The primer may include any combination of nucleotides or analogs thereof. In some embodiments, the primer is a single-stranded oligonucleotide or polynucleotide.

As used herein, the term “double-stranded,” when used in reference to a nucleic acid molecule, means that substantially all of the nucleotides in the nucleic acid molecule are hydrogen bonded to a complementary nucleotide. A partially double-stranded nucleic acid can have at least 10%, at least 25%, at least 50%, at least 60%, at least 70%, at least 80%, at least 90% or at least 95% of its nucleotides hydrogen bonded to a complementary nucleotide.

A “double-stranded oligonucleotide complex” means complex comprising, consisting of, or consisting essentially of a double-stranded oligonucleotide or a partially double stranded oligonucleotide. In embodiments, at least at least 50%, at least 60%, at least 70%, at least 80%, at least 90% or at least 95% of the nucleotides of a double stranded oligonucleotide complex are hydrogen bonded to a complementary nucleotide.

As defined herein, “sample” and its derivatives is used in its broadest sense and includes any specimen, culture and the like that is suspected of including a target nucleic acid. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual; a collection of nucleic acid samples from genetically related members; nucleic acid samples from genetically unrelated members; nucleic acid samples (matched) from a single individual such as a tumor sample and normal tissue sample; or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

As used herein, the term “adapter” and its derivatives, e.g., universal adapter, refers generally to any linear oligonucleotide which can be ligated to a target nucleic acid. In some embodiments, the adapter is substantially non-complementary to the 3′ end or the 5′ end of any target sequence present in a sample. In some embodiments, suitable adapter lengths are in the range of about 10-100 nucleotides, about 12-60 nucleotides and about 15-50 nucleotides in length. Generally, the adapter can include any combination of nucleotides and/or nucleic acids. In some embodiments, the adapter can include one or more cleavable groups at one or more locations. In some embodiments, the adapter can include a sequence that is substantially identical, or substantially complementary, to at least a portion of a primer. In some embodiments, the adapter can include a sequence that is substantially identical, or substantially complementary, to at least a portion of a surface oligonucleotide. In some embodiments, the adapter can include a barcode, also referred to as an index or tag, to assist with downstream error correction, identification, or sequencing. The terms “adaptor” and “adapter” are used interchangeably.

The term “ally-dNTP,” such as ally-thymine (ally-T), ally-cytosine (ally-C), ally-guanine (ally-G), and ally-adenine (ally-A) refer to a nucleotide that has an ally group at the 5′ carbon of the ribose or deoxyribose sugar. An ally-dNTP can be incorporated at any point in an oligonucleotide or nucleic acid. An example structure of a dinucleotide that includes an ally-T is shown below.

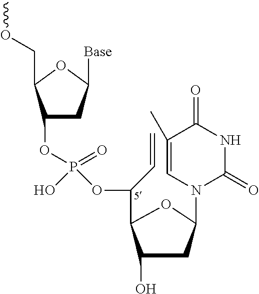

The term “surface oligonucleotide” refers to a polymeric form of a nucleotide that is attached to a surface. In some embodiments, the surface oligonucleotide is attached through the surface at the 5′ end and has a free 3′ end. The terms “P5” (SEQ ID NO: 1), “P7” (SEQ ID NO: 2), “P15” (SEQ ID NO: 3), and “P17” (SEQ ID NO: 4) may be used when referring to a surface oligonucleotide. P5, P7, P15, and P17 are described in US Patent Pub. No. US 2019/0352327. The terms “P5′” (P5 prime), “P7′” (P7 prime), “P15′” (P15 prime), and “P17′” (P17 prime) refer to the complement of P5, P7, P15, and P17 respectively. It will be understood that any suitable surface oligonucleotide can be used in the methods presented herein, and that the use of P5, P7, P15, and P17 are exemplary embodiments only. Uses of surface oligonucleotide such as P5, P7,P15, P17 on flowcells is known in the art, as exemplified by the disclosures of WO 2007/010251, WO 2006/064199, WO 2005/065814, WO 2015/106941, WO 1998/044151, and WO 2000/018957. In view of the general knowledge available and the teachings of the present disclosure, one of skill in the art will understand how to design and use sequences that are suitable for surface oligonucleotides.

As used herein, the term “universal sequence” refers to a region of sequence that is common to two or more target nucleic acids, where the molecules also have regions of sequence that differ from each other. A universal sequence that is present in different members of a collection of molecules can allow capture of multiple different nucleic acids using a population of capture nucleic acids that are complementary to a portion of the universal sequence, e.g., a universal capture binding sequence. Non-limiting examples of universal capture binding sequences include sequences that are identical to or complementary to P5 and P7 primers. Similarly, a universal sequence present in different members of a collection of molecules can allow the replication or amplification of multiple different nucleic acids using a population of universal primers that are complementary to a portion of the universal sequence, e.g., a universal primer binding site. Target nucleic acid molecules may be modified to attach universal adapters (also referred to herein as adapters), for example, at one or both ends of the different target sequences, as described herein.

As used herein, the term “different,” when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different from each other while also having a universal sequence region that are the same as each other.

As used herein, the term “nucleic acid” is intended to be consistent with its use in the art and includes naturally occurring nucleic acids and functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from adenine, thymine, cytosine or guanine and a ribonucleic acid can have one or more bases selected from uracil, adenine, cytosine or guanine. Useful non-native bases that can be included in a nucleic acid are known in the art. The term “target,” when used in reference to a nucleic acid (e.g, “nucleic acid target” or “target nucleic acid”) is intended as a semantic identifier for the nucleic acid in the context of a method or composition set forth herein and does not necessarily limit the structure or function of the nucleic acid beyond what is otherwise explicitly indicated. A “target nucleic acid” having an adapter at one or more ends, is referred to as a polynucleotide template.

In addition, the recitations herein of numerical ranges by endpoints include all numbers subsumed within that range (e.g., 1 to 5 includes 1, 1.5, 2, 2.75, 3, 3.80, 4, 5, etc.). Where a range of values is “greater than”, “less than”, etc. a particular value, that value is included within the range.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. However, it will be understood that a presented order is one embodiment of an order by which the method may carried out. Any recited single or multiple feature or aspect in any one claim may be combined or permuted with any other recited feature or aspect in any other claim or claims.

### DETAILED DESCRIPTION

Reference will now be made in greater detail to various embodiments of the subject matter of the present disclosure, some embodiments of which are illustrated in the accompanying drawings.

Presented herein are methods relating to sequencing oligonucleotides. Specifically, the present disclosure provides methods for double-stranded sequencing. “Double-stranded sequencing” refers to the sequencing of a first oligonucleotide strand of a double-stranded oligonucleotide complex. The portion of the first oligonucleotide strand being sequenced is in proximity to second strand oligonucleotide strand that is hybridized to the first strand.

Double-stranded sequencing methods may decrease the likelihood of the formation of secondary structures such as G-quadraplexes that may form when the polynucleotide template is in single strand form. As such, double-stranded sequencing methods may advantageously allow for higher sequencing accuracy relative to single stranded sequencing methods when single stranded nucleotide sequences form secondary structures that may be detrimental to sequencing.

In some embodiments, the sequencing methods of the present disclosure are particularly useful for next generation sequencing, also called massively parallel sequencing. Next generation sequencing allows many target nucleic acids to be sequenced simultaneously.

Preparation of target nucleic acids for sequencing may include one or more of (i) preparing a library of oligonucleotide templates from target nucleic acids, (ii) immobilizing the library of polynucleotide templates onto a surface, and (iii) amplifying the immobilized polynucleotide templates. The amplified polynucleotide templates may be sequenced according to the methods described herein to determine the sequence of at least a portion of the target nucleic acids.

### Preparing a Library of Polynucleotide Templates

Libraries of polynucleotide templates may be prepared in any suitable manner. In some embodiments, preparing a library of polynucleotide templates includes obtaining the target nucleic acids and ligating adapters to the target nucleic acids to create polynucleotide templates.

As used herein, the term “target nucleic acid” refers to a nucleic acid molecule where identification of at least a portion of its nucleotide sequence is desired. The target nucleic acid may be essentially any nucleic acid of known or unknown sequence. The sequence of two or more target nucleic acids in the population of target nucleic acids may be the same or different.

Sequencing may result in the determination of the sequence of a part of the target nucleic acid or the entire target nucleic acid. The target nucleic acid or a population of target nucleic acids can be derived from one or more primary nucleic acid samples. A primary nucleic acid sample may originate in double-stranded DNA (dsDNA) form (e.g., genomic DNA fragments, PCR and amplification products, and the like) or may originate in single-stranded form, as DNA or RNA that may been converted to dsDNA.

A primary target nucleic acid may be obtained from any biological sample using known, routine methods. Suitable biological samples include, but are not limited to, a blood sample, biopsy specimen, tissue explant, organ culture, biological fluid, or any other tissue or cell preparation, or fraction thereof, or derivative thereof, or isolated therefrom. In some embodiments, a primary target nucleic acid may be obtained as a sample from a human, an animal, a bacterium, a fungus, or a virus.

The target nucleic acid or a population of target nucleic acids can be derived from a primary nucleic acid sample that has been sequence specifically fragmented or randomly fragmented. For example, a fragment of genomic DNA or cDNA may be used as a target nucleic acid or a population of target nucleic acids. Random fragmentation refers to the fragmentation of a nucleic acid from a primary nucleic acid sample in a non-ordered fashion by enzymatic, chemical, or mechanical methods. Such fragmentation methods are known in the art and use standard methods (e.g., see Sambrook and Russell, Molecular Cloning, A Laboratory Manual, third edition).

Once the target nucleic acid or population of target nucleic acids are obtained, a library of polynucleotide templates for use in the provided sequencing methods may be prepared using a variety of standard techniques available and known in the art. The term “library” refers to the collection of polynucleotide templates containing known common sequences at their 3′ and/or 5′ ends, for example, by attachment of adapters. Each polynucleotide template of the library includes one or more target nucleic acids. Exemplary methods of polynucleotide template preparation include, but are not limited to, those described in Bentley et al., Nature 456:49-51 (2008); U.S. Pat. No. 7,115,400; and U.S. Patent Application Publication Nos. 2007/0128624; 2009/0226975; 2005/0100900; 2005/0059048; 2007/0110638; and 2007/0128624, each of which is herein incorporated by reference in its entirety.

For the sequencing methods of the present disclosure, the polynucleotide templates include adapters that are ligated to the 5′ and/or 3′ ends of the target nucleic acid. Methods for attaching adapters to one or both ends of a target nucleic acid are known to the person skill in the art. The attachment can be through standard library preparation techniques using, for example, ligation (U.S. Pat. Pub. No. 2018/0305753), or tagmentation using transposase complexes (Gunderson et al., WO 2016/130704).

Adapters include one or more known sequences. When the polynucleotide template includes adapters with known sequences on the 5′ and/or 3′ ends, the known sequences may be the same or different. Consistent with the methods of present disclosure, known adapter sequence located on the 5′ and/or 3′ ends of the polynucleotide templates are capable of hybridizing to one or more surface oligonucleotides that are immobilized on a surface. For instance, for use with a surface that includes P5 and P7 surface oligonucleotides, the adapters may include P5′ or a P7′ sequence or derivative thereof. The P5 surface oligonucleotide may hybridize with the P5′ adapter sequence and the P7 surface oligonucleotide may hybridize with the P7′ adapter sequence. Optionally, polynucleotide templates may include one or more detectable labels. The one or more detectable labels may be attached to the polynucleotide template at the 5′ end, at the 3′ end, and/or at any nucleotide position within the polynucleotide template, for example, within the adapter sequence.

The adapters may further include one or more universal sequences. A universal sequence is a region of nucleotide sequence that is common to, e.g., shared by, two or more polynucleotide templates, where the two or more polynucleotide templates also have regions of sequence differences (e.g., the target nucleic acid). A universal sequence that may be present in different members of a library of polynucleotide templates may allow the replication or amplification of multiple different sequences using a single universal primer that is complementary to the universal sequence. Similarly, at least one, two (e.g., a pair), or more universal sequences that may be present in different members of a library of polynucleotide templates may allow the replication or amplification of multiple different sequences using at least one, two (e.g., a pair), or more single universal primers that are at least partially complementary to the universal sequences. Thus, a universal primer includes a sequence that may hybridize specifically to such a universal sequence.

The adapters may also include one or more index sequences. An index can be used as a marker characteristic of the source of particular target nucleic acid (U.S. Pat. No. 8,053,192). Generally, the index is a synthetic sequence of nucleotides that is part of the adapter which is added to the target nucleic acids as part of the library preparation step. Accordingly, an index is a nucleic acid sequence which is attached to each of the target nucleic acids of a particular sample, the presence of which is indicative of, or is used to identify, the sample or source from which the target nucleic acids were isolated. In some embodiments, a dual index system may be used. In a dual index system, the adapter attached to target nucleic acids includes two different index sequences, for example as described in U.S. Pat. Nos. 10,975,430; 10,995,369; 10,934,584; and U.S. Pat. Pub. No. 2018/0305753.

In some embodiments, the adapters comprise a cleavage site. The adapters may include any suitable cleavage site. Examples of suitable cleavage sites include abasic cleavage sites, chemical cleavage sites, ribonucleotide cleavage sites, photochemical cleavage sites, hemimethylated DNA cleavage sites, nicking endonuclease cleavage sites, and restriction enzyme cleavage sites.

The polynucleotide templates may also be modified to include any nucleic acid sequence desirable using standard, known methods. The modifications may be incorporated as a part of the adapter or separately, for example, prior to adapter ligation. Such additional sequences may include, but are not limited to, restriction enzyme sites, non-natural nucleotides, modified nucleic acids, and combinations thereof. Examples of unnatural or modified nucleic acids include, but are not limited to, deoxyuridine (U), 8-oxo-guanine (8-oxo-G), hemimethylated sequences, ally-dNTPs (e.g., ally-T, ally-C, ally-G, and ally-A), and deoxyinosine.

In some embodiments, the polynucleotide templates may include one or more modified nucleotides that enhance base pair binding, relative to a natural nucleotide, to a nucleotide of the template polynucleotide. The modifications may be incorporated as a part of the adapter or separately, for example, prior to adapter ligation. Modified nucleotides are known and include, for example, locked nucleotides (LNAs) and bridged nucleotides (BNAs). LNAs and BNAs, as well as oligonucleotides containing LNAs and BNAs, are commercially available. The following publications provide additional information regarding BNAs: (1) Obika, S., et al., (1997), “Synthesis of 2′-0,4′-C-methyleneuridine and -cytidine. Novel bicyclic nucleosides having a fixed C3, -endo sugar puckering,” Tetrahedron Letters. 38 (50): 8735; (2) Obika, S., et al., (2001), “3′-amino-2′, 4′-BNA: Novel bridged nucleic acids having an N3′-->P5′ phosphoramidate linkage,” Chemical communications (Cambridge, England) (19): 1992-1993; (3) Obika, S., et al., (2001), “A 2′, 4′-Bridged Nucleic Acid Containing 2-Pyridone as a Nucleobase: Efficient Recognition of a C-G Interruption by Triplex Formation with a Pyrimidine Motif,” Angewandte Chemie International Edition. 40 (11): 2079; (4) Morita, K., et al., (2001), “2′-O,4′-C-ethylene-bridged nucleic acids (ENA) with nuclease-resistance and high affinity for RNA,” Nucleic Acids Research. Supplement. 1 (1): 241-242; (5) Hari, Y., et al., (2003), “Selective recognition of CG interruption by 2′, 4′-BNA having 1-isoquinolone as a nucleobase in a pyrimidine motif triplex formation,” Tetrahedron. 59 (27): 5123; (6) Rahman, S. M. A., et al., (2007), “Highly Stable Pyrimidine-Motif Triplex Formation at Physiological pH Values by a Bridged Nucleic Acid Analogue,” Angewandte Chemie International Edition. 46 (23): 4306-4309. LNAs monomers include an additional bridge that connects the 2′ oxygen and the 4′ carbon of a ribose moiety to “lock” the ribose in the 3′-endo conformation. Preferably, the modified nucleotides form standard Watson-Crick base pairs. For example, LNA bases form standard Watson-Crick base pairs but the locked configuration increases the rate and stability of the base pairing (Jepsen et al., Oligonucleotides, 14, 130-146 (2004)).

In some embodiments, the polynucleotide templates may include non-natural backbone linkages such as a diol or disulfide; photo-cleavable spacer group; or any combination thereof. The modifications may be incorporated as a part of the adapter, or separately prior to adapter ligation.

In some embodiments, prior to or after adapter ligation, the polynucleotides templates are amplified. Amplification may be accomplished through any known amplification process known in the art, for example, solid-phase amplification, polony amplification, colony amplification, polymerase chain reaction (PCR) such as emulsion PCR, bead rolling circle amplification (RCA), surface RCA, or surface exponential strand displacement (SDA). Amplification can be thermal or isothermal.

Immobilization of the Library of Polynucleotide Templates onto a Surface

As used herein the term surface refers to a substrate for attaching nucleic acids. A surface is made of material that has a rigid or semi-rigid structure to which a polynucleotide can be attached or upon which nucleic acids can be synthesized and/or modified. Surfaces can include any resin, gel, bead, well, column, chip, flow cell, membrane, matrix, plate, filter, glass, controlled pore glass (CPG), polymer support, membrane, paper, plastic, plastic tube or tablet, plastic bead, glass bead, slide, ceramic, silicon chip, multi-well plate, nylon membrane, fiber optic, and PVDF membrane. In some embodiments, the surface is within or a part of a flow cell.

The surface includes a population of surface oligonucleotides that are immobilized on the surface. The surface oligonucleotides may be covalently attached to the surface. The surface oligonucleotides are generally configured to bind or hybridize to a portion of a polynucleotide template, particularly to a portion of the adapter of the polynucleotide template. The surface oligonucleotides are attached to the surface at the 5′ end and have a free 3′ end. The population of surface oligonucleotides may include a population of a first surface oligonucleotide and a population of a second surface oligonucleotide where the first surface oligonucleotide and the second surface oligonucleotide have different sequences. In some embodiments, the first surface oligonucleotide includes the sequence of P7 (SEQ ID NO. 1). In some embodiments, the second surface oligonucleotide includes the sequence of P5 (SEQ ID NO. 2). In some embodiments, the second surface oligonucleotide includes the sequence of P15 (SEQ ID NO. 3). The P7, P5, and P15 surface oligonucleotides are configured to hybridize with the P7′, P5′, and P15′ sequences of adapters attached to template polynucleotides. Uses of surface oligonucleotides such as P5 and P7 on flow cells is known in the art, as exemplified by the disclosures of WO 2007/010251, WO 2006/064199, WO 2005/065814, WO 2015/106941, WO 1998/044151, and WO 2000/018957. P7, P5, and P15 surface oligonucleotides are also described in, for example, US 2019/0352327, which is hereby incorporated by reference in its entirety. In some embodiments, additional populations of surface oligonucleotides having sequences different from the first and second surface oligonucleotides may be present. Attachment of the surface oligonucleotides to the surface can be accomplished through any method known in the art, for example, such as those described in U.S. Pat. No. 8,895,249, WO 2008/093098, and US. Pat. Pub. No. 2011/0059865 A1, amongst others. In some embodiments, the surface oligonucleotides may include one or more unnatural or modified nucleic acids, unnatural backbone linkages, restriction enzyme sequences, or any combination thereof, such as those described elsewhere herein.

The polynucleotide templates are immobilized on the surface through hybridization of the adapter portion that is configured to bind to at least one surface oligonucleotide. For example, if the population of first surface oligonucleotides includes the P5 sequence, polynucleotide templates that include the P5′ sequence in the adapter region may hybridize to the first surface oligonucleotide. If the population of first surface oligonucleotides includes the P7 sequence, polynucleotide templates that include the P7′ sequence in the adapter region may hybridize to the first surface oligonucleotide. If the population of first surface oligonucleotides includes the P15 sequence, polynucleotide templates that include the P15′ sequence in the adapter region may hybridize to the first surface oligonucleotide.

The surface oligonucleotides may be used as primers for chain extension or amplification using as templates the hybridized polynucleotide templates.

### Surface Amplification of the Polynucleotide Templates

The polynucleotide templates may be amplified on the surface to which they are immobilized. Polynucleotide template amplification includes the process of amplifying or increasing the numbers of a polynucleotide templates and/or of a complement thereof, by producing one or more copies of the template and/or its complement. Amplification may be carried out by a variety of known methods under conditions including, but not limited to, thermocycling amplification or isothermal amplification. For example, methods for carrying out amplification are described in U.S. Pat. Pub. No. 2009/0226975; WO 98/44151; WO 00/18957; WO 02/46456; WO 06/064199; and WO 07/010251; which are incorporated by reference herein in their entireties.

Briefly, amplification may occur on the surface to which the polynucleotide templates are immobilized. This type of amplification can be referred to as solid phase amplification, which when used in reference to polynucleotide templates, refers to any polynucleotide template amplification reaction carried out on or in association with a surface. Typically, all or a portion of the amplified products are synthesized by extension of a primer that is immobilized on the surface.

Solid-phase amplification may include a polynucleotide template amplification reaction including only one species of surface oligonucleotide immobilized to a surface. Alternatively, the surface may comprise a plurality of first and second different immobilized surface oligonucleotide species. Solid phase polynucleotide template amplification reactions generally include at least one of two different types of nucleic acid amplification, interfacial or surface (or bridge) amplification. For instance, in interfacial amplification the surface includes a polynucleotide template that is indirectly immobilized to the solid support by hybridization to an immobilized surface oligonucleotide, the immobilized surface oligonucleotide may be extended in the course of a polymerase-catalyzed, template-directed elongation reaction (e.g., primer extension) to generate an immobilized polynucleotide that remains attached to the solid support. After the extension phase, the polynucleotides (e.g., polynucleotide template and its complementary product) may be denatured such that the template polynucleotide is released into solution and made available for hybridization to another immobilized primer. The polynucleotide template may be made available in 1, 2, 3, 4, 5 or more rounds of primer extension or may be washed out of the reaction after 1, 2, 3, 4, 5 or more rounds of primer extension.

In surface (or bridge) amplification, an immobilized polynucleotide template hybridizes to a surface oligonucleotide immobilized on a surface. The 3′ end of the immobilized polynucleotide template provides the template for a polymerase-catalyzed, template-directed elongation reaction (e.g., primer extension) extending from the immobilized surface oligonucleotide. The resulting double-stranded product “bridges” the two surface oligonucleotides and both strands are covalently attached to the support. In the next cycle, following denaturation that yields a pair of single strands (the immobilized polynucleotide template and the extended-primer product) immobilized to the surface, both immobilized strands can serve as templates for new primer extension. Examples of bridge amplification can be found in U.S. Pat. Nos. 7,790,418; 7,972,820; WO 2000/018957; U.S. Pat. No. 7,790,418; and Adessi et al., Nucleic Acids Research (2000): 28(20): E87).

In some embodiments, after bridge amplification and while the double-stranded bridge complex exists, the surface may be treated with an exonuclease. The exonuclease will remove at least a portion of surface oligonucleotides that are not participating in a double-stranded bridged structure. The exonuclease may completely remove individual surface oligonucleotides or remove portions of individual surface oligonucleotides. Treating the surface with an exonuclease prior to applying the sequencing methods of the present disclosure may result in a lower background signal during sequencing.

Any suitable exonuclease may be used. Examples of suitable exonucleases include Exonuclease I, Exonuclease T, and Exonuclease VII (all are available from New England Biolabs, MA). Preferably, the exonuclease has a high specificity for single stranded DNA over double-stranded DNA.

Amplification may be used to produce colonies of immobilized polynucleotide templates. For example, the methods can produce clustered arrays of polynucleotide template colonies, analogous to those described in U.S. Pat. Nos. 7,115,400; 7,985,565; WO 00/18957; and WO 98/44151, which are incorporated by reference herein in their entireties. “Clusters” and “colonies” are used interchangeably and refer to a plurality of copies of a polynucleotide template having the same sequence and/or complements thereof attached to a surface. Typically, the cluster comprises a plurality of copies of a polynucleotide template having the same sequence and/or complements thereof, attached via their 5′ end to the surface. The copies of polynucleotide templates making up the clusters may be in a single or double-stranded form.

The plurality of polynucleotide templates may be in a cluster, each cluster containing polynucleotide templates of the same sequence. A plurality of clusters can be sequenced, each cluster comprising polynucleotide templates of the same sequence. Optionally, the sequence of the polynucleotide templates in a first cluster is different from the sequence of the polynucleotide templates of a second cluster. Optionally, the cluster is formed by annealing a polynucleotide template to a primer on a surface and amplifying the polynucleotide template under conditions to form the cluster that includes the plurality of polynucleotide templates of the same sequence. Amplification can be thermal or isothermal.

Each colony may include a plurality of polynucleotide templates of the same sequences. In some embodiments, the sequence of the polynucleotide templates of one colony is different from the sequence of the polynucleotide templates of another colony. Thus, each colony comprises polynucleotide templates having different target nucleic acid sequences. All the immobilized polynucleotide templates in a colony are typically produced by amplification of the same polynucleotide template. In some embodiments, it is possible that a colony of immobilized polynucleotide templates includes one or more primers without an immobilized polynucleotide template to which another polynucleotide of different sequence may bind upon additional application of solutions containing free or unbound polynucleotide templates.

### Double-Stranded Sequence of Target Nucleic Acids

The present disclosure is directed to, among other things, methods for sequencing oligonucleotide strands that contain one or more target nucleic acids. Particularly, the present disclosure is directed at double-stranded sequencing of at least one oligonucleotide strand of a double-stranded oligonucleotide complex. In some embodiments, at least one of the oligonucleotide strands of the double-stranded oligonucleotide complex is immobilized on a surface. Accordingly, the sequencing methods may be carried out on oligonucleotides that are polynucleotide templates that have been immobilized to a surface and amplified as described above. The oligonucleotides may be polynucleotide templates and include adapter nucleic acid sequences on the 5′ end, 3′ end, or both.

For double-stranded sequencing, a sequencing complex is provided. Sequencing complexes include a double-stranded oligonucleotide complex and a sequencing primer. The double-stranded oligonucleotide complex includes a first oligonucleotide strand bound to the surface and a second oligonucleotide strand at least partially hybridized to a first portion of the first oligonucleotide strand, the first oligonucleotide strand including the target nucleic acid. The sequencing primer is hybridized to a second portion of the first oligonucleotide strand. In some embodiments, the double-stranded oligonucleotide complex is, for example, a double-stranded bridge structure created during bridge amplification. In other embodiments, the double-stranded complex is created independently of bridge amplification. In some embodiments, the sequencing primer is derived from the second oligonucleotide strand. In other embodiments, the sequencing primer is independent of the second oligonucleotide strand.

The sequencing methods of the present disclosure preferably use sequencing by synthesis (SBS) to elucidate the nucleotide sequence of regions of interest on the polynucleotide templates. SBS techniques include, but are not limited to, ISEQ sequencing systems, the MINISEQ sequencing systems, the MISEQ sequencing systems, and the NEXTSEQ sequencing systems (Illumina Inc., San Diego, CA); and the True Single Molecule Sequencing (tSMS)™ systems (Helicos BioSciences Corporation, Cambridge, MA). In the SBS technique, a number of sequencing by synthesis reactions are used to elucidate the identity of a plurality of bases at target positions within a target sequence. In some embodiments that include SBS, the reactions rely on the use of a target nucleic acid sequence having at least two domains; a first domain to which a sequencing primer will hybridize; and an adjacent second domain, for which sequence information is desired.

After formation of an initial sequencing complex, a chain extension enzyme may be used to add deoxynucleotide triphosphates (dNTPs) to the sequencing primer, and each addition of dNTPs may be read to determine the identity of the added dNTP. This may proceed for many cycles. The sequence for which the nucleotide identity is determined is generally termed a “read.” Read lengths may be greater than 5, greater than 10, greater than 20, greater than 50, greater than 100, greater than 200, greater than 300, or greater than 400 nucleotides in length.

In some SBS embodiments, an oligonucleotide strand hybridizes with a sequencing primer and incubated in the presence of a polymerase and one or more labeled nucleotides that include a 3′ blocking group. Examples of labeled nucleotides that include a blocking group can be found in WO 2004/018497. The sequencing primer is extended such that the labeled nucleotide is incorporated. The presence of the blocking group permits only one round of incorporation, that is, the incorporation of a single nucleotide. The presence of the label permits identification of the incorporated nucleotide. In some embodiments, the label is a fluorescent label. A plurality of homogenous single nucleotide bases can be added during each cycle, such as used in the True Single Molecule Sequencing (tSMS)™ systems (Helicos BioSciences Corporation, Cambridge, MA). Alternatively, all four nucleotide bases can be added during each cycle simultaneously, such as used in the ISEQ sequencing systems, the MINISEQ sequencing systems, the MISEQ sequencing systems, and the NEXTSEQ sequencing systems (Illumina Inc., San Diego, CA), particularly when each base is associated with a distinguishable label. After identifying the incorporated nucleotide by its corresponding label, both the label and the blocking group can be removed, thereby allowing a subsequent round of incorporation and identification. Determining the identity of the added nucleotide base includes, in some embodiments, repeated exposure of the newly added labeled bases to a light source that can induce a detectable emission due to the addition of a specific nucleotide. In some embodiments, the label is a fluorescent label.

In some embodiments, the nucleotides used in SBS do not include a label, for example when pyrosequencing is used. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Because the incorporation of any dNTP into a growing chain releases pyrophosphate, the four dNTP bases must be added to the system in separate steps. Useful fluidic systems, detectors, and procedures that can be used for application of pyrosequencing to arrays of the present disclosure are described, for example, in WO2012058096A1; US Pat. Pub. No. 2005/0191698 A1; U.S. Pat. Nos. 7,595,883; and 7,244,559.

Sequencing-by-ligation SBS reactions such as those described in Shendure et al. Science 309:1728-1732 (2005); U.S. Pat. Nos. 5,599,675; and 5,750,341 may also be used. Some embodiments can include sequencing-by-hybridization procedures as described, for example, in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977. In both sequencing-by-ligation and sequencing-by-hybridization procedures, oligonucleotides that are present at sites of an array are subjected to repeated cycles of oligonucleotide delivery and detection. Fluidic systems for SBS methods can be readily adapted for delivery of reagents for sequencing-by-ligation or sequencing-by-hybridization procedures. Typically, the oligonucleotides are fluorescently labeled and can be detected using fluorescence detectors similar to those described with regard to SBS procedures herein or in references cited herein.

Some embodiments can use methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008).

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in U.S. Pat. Nos. 8,262,900; 7,948,015; US Pat. Pub. 2010/0137143 A1; or U.S. Pat. No. 8,349,167.

The sequencing methods disclosed herein are particularly useful when used in conjunction with SBS. In addition, the sequencing methods described herein may be particularly useful for sequencing from an array clusters of oligonucleotides, where multiple sequences can be read simultaneously from multiple clusters on the array since each nucleotide at each position can be identified based on its identifiable label. Exemplary methods are described in U.S. Pat. Nos. 7,754,429; 7,785,796; and 7,771,973, each of which is incorporated herein by reference.

In some embodiments, where the oligonucleotide includes one or more index sequences, the index sequences may be sequenced using SBS.

In some embodiments, SBS involves several rounds of incorporation of nucleotides for which the identity of the incorporated nucleotides are not determined. Such rounds of incorporation may be referred to as “dark cycles.” Dark cycling involves the sequential incorporation of nucleotides containing a 5′ blocking group and subsequent blocking group removal. Dark cycles may be used to skip the reading of index sequences, universal sequences, and/or any other sequence where the identity is not desired to be determined. Each cycle of a dark cycle includes the incorporation of a nucleotide. Any suitable number of dark cycles of incorporation may be performed to effectively reach the portion of the polynucleotide template where determining the nucleotide sequence is desired. For example, 2 to 150 dark incorporation cycles may be performed, such as 3 to 100, 5 to 50, or 6 to 25 dark cycles. The sequence of the polynucleotide template strand to which the extended sequencing primer is complementary during the dark cycles is preferably known. Once the appropriate number of dark cycles of incorporation are performed, SBS (determining the identity of the nucleotides incorporated in subsequent cycles) may be performed.

### Strand Displacement and Nick Translation

Double-stranded sequencing is the sequencing of an oligonucleotide strand of a double-stranded oligonucleotide complex. Sequencing of the oligonucleotide strand of the double-stranded oligonucleotide complex may proceed via strand displacement, nick translation, a combination thereof, or any other suitable mechanism.

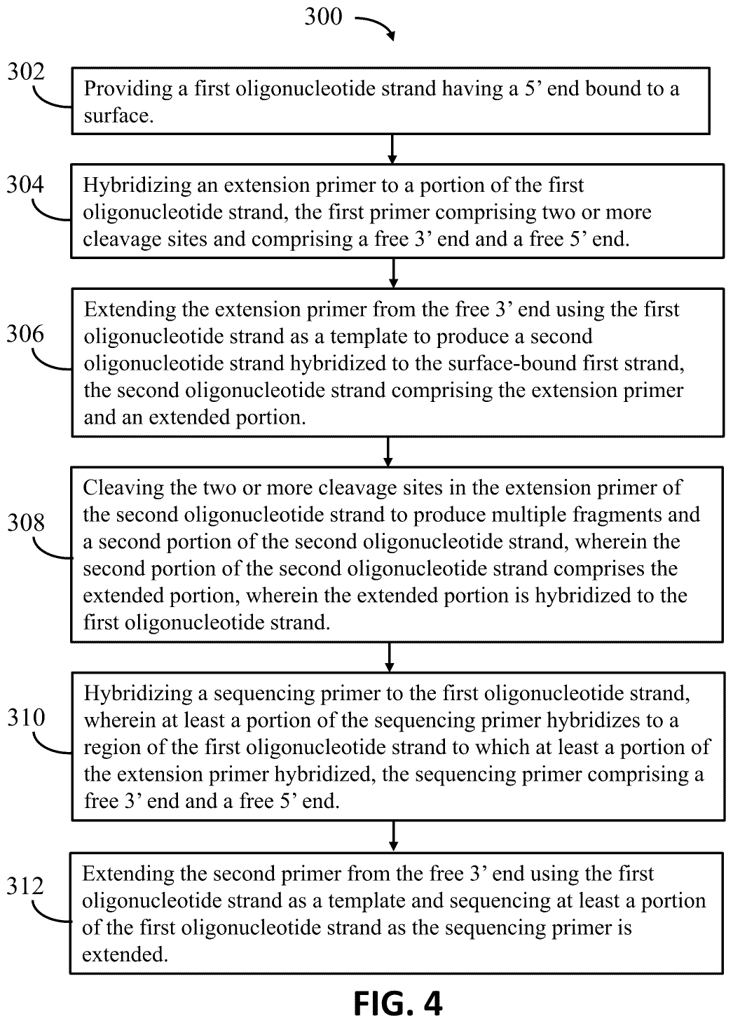

Double-stranded sequencing via strand displacement involves de-hybridizing a second oligonucleotide strand from a first oligonucleotide strand of a double-stranded oligonucleotide complex as a nascent third oligonucleotide strand is formed. FIG. 4 illustrates sequencing via strand displacement from a sequencing complex SC. The sequencing complex includes a first oligonucleotide strand AA, a second oligonucleotide strand BB, and a primer EE. The second oligonucleotide strand BB has a free 5′ and at least a portion of the second oligonucleotide strand BB is hybridized to at least a portion of the first oligonucleotide strand AA in a double-stranded oligonucleotide complex. The primer EE is hybridized to a portion of the first oligonucleotide strand AA and has a free 3′ end. During SBS, the primer EE is extended in the 5′ to 3′ direction using the first oligonucleotide strand AA as a template, thereby creating a third oligonucleotide strand FF. As the third oligonucleotide strand FF is extended, it displaces a 5′ portion of the second oligonucleotide strand BB. The displaced portion CC of the second oligonucleotide strand BB may be referred to as a flap or an overhang.

Double-stranded sequencing via nick translation involves removing nucleotides from a second oligonucleotide strand of a double-stranded oligonucleotide complex and replacing the removed nucleotides with newly incorporated nucleotides on a growing third oligonucleotide strand, using the first oligonucleotide strand as a template. FIG. 4 illustrates sequencing via nick translation from the sequencing complex SC. In nick translation, the second oligonucleotide strand BB is nicked by a nickase. Following nicking, nucleotides are removed from a 5′ end portion of the nicked second oligonucleotide strand GG. The removed nucleotides are replaced with newly incorporated nucleotides DD on the 3′ end of a sequencing primer EE or of a growing third oligonucleotide strand FF in which the first strand AA is used as a template. In some embodiments, a 5′ end of the sequencing primer EE is attached to a surface to which a 3′ end of the first oligonucleotide strand is attached. In some embodiments, the sequencing primer EE has a free 5′ end; that is, the sequencing primer is not attached to a surface. In nick translation, the growing third oligonucleotide strand FF and the nicked second oligonucleotide strand GG form a double-stranded complex with the unnicked first oligonucleotide strand AA. The growing third oligonucleotide strand FF and the nicked second oligonucleotide strand GG are hybridized to the first oligonucleotide strand AA.

In nick translation, the 3′ end of the growing third oligonucleotide strand FF and the 5′ end of the nicked second oligonucleotide strand GG are zero to fifty (e.g., zero to one, zero to two, zero to three, zero to four, zero to five, zero to ten, zero to fifteen, etc.) nucleotides apart as measured by the nucleotides on the unnicked first oligonucleotide strand AA. The 5′ end of the nicked second oligonucleotide strand GG and the 3′ end of the growing third oligonucleotide strand FF are considered to be zero nucleotides apart when only a nick, with no intervening gap or with no flap, in the strand separates 5′ end of the nicked second oligonucleotide strand GG and the 3′ end of the growing third oligonucleotide strand FF.

In embodiments where the number of nucleotides between 5′ end of the nicked second oligonucleotide strand GG and the 3′ end of the growing third oligonucleotide strand FF is greater than zero, a flap nuclease may remove the nucleotides from the 5′ end portion of the nicked second oligonucleotide strand GG. A flap nuclease is a nuclease that prevents formation of a flap or cleaves or removes at least a portion of a flap that is or would otherwise be formed due to the addition of nucleotides to the 3′ end of the growing third oligonucleotide strand FF. In some embodiments, the flap nuclease has 5′ to 3′ exonuclease activity. In some embodiments, the flap nuclease has endonuclease activity. In some embodiments, the flap nuclease having endonuclease or exonuclease activity recognizes a nick or break in a single strand of the double-stranded complex and introduces a nick in the nicked strand at a location 3′ of the recognized nick or break. A polymerase may be used to introduce the newly incorporated nucleotides to the 3′ end of a primer EE or a 3′ end of the growing third oligonucleotide strand FF. The nicked second strand GG may be nicked before, during, or after incorporation of a nucleotide to the growing third oligonucleotide strand FF. In some embodiments, the nicked second strand GG is nicked before, during, or after incorporation of each nucleotide to the growing third oligonucleotide strand FF. In some embodiments, the nicked second strand GG is nicked after incorporation of multiple nucleotides to the growing third oligonucleotide strand FF has occurred.

During nick translation, a flap nuclease (i.e., a domain or protein having flap nuclease activity) may be added or may be present during certain steps of the method (e.g., during sequencing and/or chain extension that is independent from sequencing) to facilitate removal of the nucleotides on the impeding strand (i.e., the nicked second oligonucleotide strand GG). As used herein, a “flap nuclease” is a protein, or domain thereof, that can introduce a break in, or remove nucleotides from, one strand of a double-stranded oligonucleotide complex. A flap nuclease may be a flap nicking enzyme. In some embodiments, the flap nuclease comprises a domain of a protein than includes domains having other enzymatic activity.

The flap nuclease may have exonuclease or endonuclease activity. In some embodiments, the flap nuclease has exonuclease activity. In some embodiments, the flap nuclease has 5′ to 3′ exonuclease activity. The use of a flap nuclease having 5′ to 3′ exonuclease activity may allow for the sequential 5′ to 3′ removal of nucleotides on the impeding strand. In some embodiments, the flap nuclease has endonuclease activity. In some embodiments, the flap nuclease having endonuclease activity removes two or more nucleotides from the impeding strand simultaneously.

Any suitable flap nuclease may be used. A flap nuclease may be a flap nuclease that is found in nature or a synthetically evolved protein that is designed to have flap nuclease activity. Examples of naturally occurring flap nuclease includes full-length or small subunits from the PolA family of DNA polymerases such as Taq DNA polymerase (e.g., amino acids 1-305 or amino acids 1-292; Bst DNA polymerase (e.g., amino acids 1-304); Flap Endonuclease I (FEN1); GINS-associated nuclease (GAN); RecJ family of exonucleases; lambda exonuclease; and combinations thereof. Examples of evolved flap nucleases include, for example RecJF. Table 1 gives examples of flap nucleases. Although a specific organism is shown for some of the flap nucleases in Table 1, the same or similar flap nuclease may be isolated from a different organism. In some embodiments, the flap nuclease includes the Taq DNA polymerase or a portion thereof that has flap nuclease activity. In some embodiments, the flap nuclease includes the Bst DNA polymerase or a portion thereof that has flap nuclease activity. In some embodiments, the flap nuclease includes FEN1 or a portion thereof that has flap nuclease activity. In some embodiments, the flap nuclease includes GAN or a portion thereof that has flap nuclease activity.

TABLE 1

| | | Protein Data Bank | |
|---|---|---|---|
| Flap | UniProt | Number or relevant | |
| Nuclease | Number | reference | Available From |
| Taq DNA | P19821 | 1TAQ | New England |
| polymerase | | | Biolabs |
| Bst DNA | P52026 | 6MU4 | New England |
| polymerase | | | Biolabs |
| FEN1 | C5A639 | — | New England |
| | (Thermococcus | | Biolabs |
| | Gammatolerans) | | |
| GAN | Q5JGLO | 5GHS | — |
| | (Thermococcus | | |
| | Kodakarensis) | | |
| GAN | NCBI number | — | — |
| | AHL22101 | | |
| | (Thermococcus | | |
| | Nautili) | | |
| RecJ | Q5SJ47 | 2ZXO | — |
| | (Thermus | | |
| | Thermophilus) | | |
| Lambda | P03697 | 4WUZ | New England |
| exonuclease | | | Biolabs |
| RecJF | — | Lovett, S. T., Kolodner, | New England |
| | | R. D. (1989). Proc. Natl. | Biolabs |
| | | Acad. Sci. USA. 86, | |
| | | 2627-2631. | |
| Bst 2.0 | — | — | New England |
| | | | Biolabs |

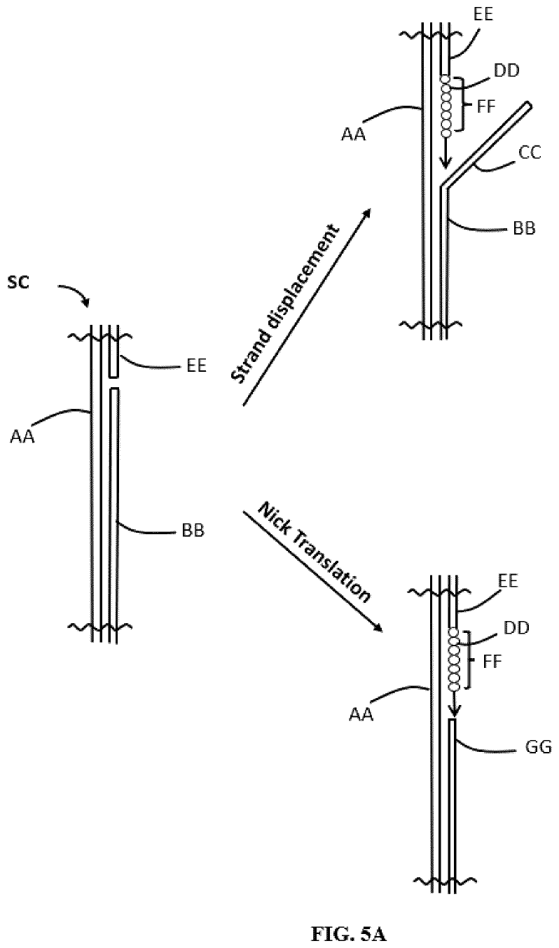

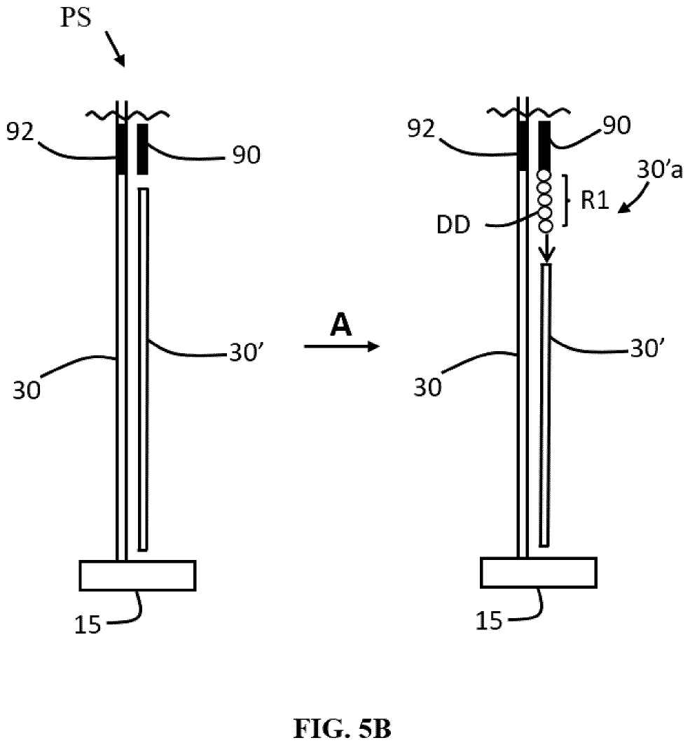

In some embodiments of nick translation described herein, a flap nuclease is added or is present at a step during each sequencing cycle. In embodiments, a flap nuclease is added after a number of sequencing cycles. In embodiments, a protein comprising polymerase activity for use in a sequencing cycle also comprises flap nuclease activity. In some embodiments, a protein comprising DNA polymerase activity also includes flap nuclease activity. In some embodiments, the protein comprising polymerase activity and a flap nuclease activity is a naturally occurring protein. In some embodiments, the protein comprising polymerase activity and a flap nuclease activity is a naturally occurring protein that has been modified to eliminate or reduce active domains that might otherwise interfere with a nick translation process described herein. In some embodiments, the protein comprising polymerase activity and a flap nuclease activity is a protein in which one or more domains comprising polymerase activity are coupled to one or more domains having flap nuclease activity. In some embodiments, the protein comprising polymerase activity and a flap nuclease activity is a fusion protein. In some embodiments, a flap nuclease may prevent the formation and/or remove an impeding strand flap from the oligonucleotide strand that was a part of the double-stranded oligonucleotide complex of the sequencing complex but is not being sequenced (e.g., the nicked second oligonucleotide strand GG in FIG. 5A). In some such embodiments, a flap nuclease is added or is present during each SBS cycle. In some such embodiments for every SBS incorporation cycle where a nucleotide is added onto the growing third oligonucleotide strand FF, a flap nuclease removes one or more nucleotide on the nicked second oligonucleotide strand GG. Thus, a flap nuclease may be used to avoid the formation of a displaced strand (i.e., a flap), which forms in the double-stranded surface sequencing by displacement methods of the present disclosure.

In some embodiments, the flap nuclease is added after a number of SBS cycles. In such embodiments, a portion of the second oligonucleotide strand BB is displaced by the growing third oligonucleotide strand FF prior to cleavage and removal; that is, a small flap is allowed to form. For example, several cycles of SBS incorporation may be run via the double-stranded sequencing via displacement methods of the present disclosure. After a predetermined number of SBS cycles where a flap has formed, a flap nuclease may be introduced to nick the second oligonucleotide strand BB forming a nicked second oligonucleotide strand GG such that the displaced portion of the second oligonucleotide strand BB is cleaved. A flap nuclease may be introduced at any suitable interval during the SBS process. For example, a flap nuclease may be introduced after or during every 2 SBS cycles, every 4 SBS cycles, every 6 SBS cycles, every 8 SBS cycles, every 10 SBS cycles, every 20 SBS cycles, every 30 SBS cycles, every 40 SBS cycles, every 50 SBS cycles, and so on.

In some embodiments, a flap nuclease is operably linked to a second protein. In embodiments, where the flap nuclease is operably linked to a second protein, the molecule may be referred to as a flap nuclease—second protein construct, or simply, construct. In some embodiments, the second protein may be, for example, a polymerase, a DNA binding domain, or both. As used herein, the term “operably linked” refers to a direct or indirect covalent linking between the second protein and the flap nuclease. Thus, a flap nuclease and a second protein that are operably linked may be directly covalently coupled to one another. Conversely, a flap nuclease and a second protein that are operably linked may be connected by mutual covalent linking to an intervening component (e.g., a flanking sequence or linker).

The flap nuclease and the second protein may be operably linked through one or more linkers. The term “linker” as used herein refers to any bond, small molecule, peptide sequence, or other vehicle that covalently links the flap nuclease and the second protein. Linkers are classified based on the presence of one or more chemical motifs such as, for example, including a disulfide group, a hydrazine group or peptide (cleavable), or a thioester group (non-cleavable). Linkers also include charged linkers, and hydrophilic forms thereof as known in the art.

Suitable linkers for linking the flap nuclease and the second protein include a peptide linker such as a natural linker, an empirical linker, or a combination of natural and/or empirical linkers. Natural linkers are derived from the amino acid linking sequence of multi-domain proteins, which are naturally present between protein domains. Properties of natural linkers such as, for example, length, hydrophobicity, amino acid residues, and/or secondary structure can be exploited to confer desirable properties to a multi-domain compound that includes natural linkers connecting the flap nuclease and the second protein. In some embodiments, the linker is an empirical linker. In some embodiments, the empirical linkers comprise a flexible linker, a rigid linker, or a cleavable linker. Flexible linkers can provide a certain degree of movement or interaction at the joined components. Flexible linkers typically include small, non-polar (e.g., Gly) or polar (e.g., Ser or Thr) amino acids, which provide flexibility, and allow for mobility of the connected components. Rigid linkers can successfully keep a fixed distance between the flap nuclease and the second protein to maintain their independent functions, which can provide efficient separation of the flap nuclease and the second protein and/or sufficiently reduce interference between the flap nuclease and the second protein. Examples of peptide linkers include GGGGSGGGGSGGGGS (SEQ ID NO. 5), AALGGAAAAAAS (SEQ ID NO. 6), ALEEAPWPPPWGA (SEQ ID NO. 7), and GCGGCATTAGGTGGTGCAGCAGCCGCGGCAGCGTCG (SEQ ID NO: 10). In some embodiments, the linker is SEQ ID NO: 5. In some embodiments, the linker is SEQ ID NO: 6. In some embodiments, the linker is SEQ ID NO: 7. In some embodiments, the linker is SEQ ID NO: 10. In some embodiments, the flap nuclease and second protein construct include a linker of the sequence of SEQ ID NO. 5, SEQ ID NO. 6, SEQ ID NO. 7, or SEQ ID NO.10.

In some embodiments, the natural linker or empirical linker is covalently attached to the second protein, the flap nuclease, or both, using bioconjugation chemistries. Bioconjugation chemistries are well known in the art and include but are not limited to, click reactions, NHS-ester ligation, isocyanate ligation, isothiocyanate ligation, benzoyl fluoride ligation, maleimide conjugation, iodoacetamide conjugation, 2-thiopyridine disulfide exchange, 3-arylpropiolonitrile conjugation, diazonium salt conjugation, PTAD conjugation, and Mannich ligation.

In some embodiments, the natural linker or empirical linker, the flap nuclease, the second protein, or any combinations thereof, may include one or more unnatural amino acids that allow for bioorthogonal conjugation reactions. As used herein, “bioorthogonal conjugation” refers to a conjugation reaction that uses one or more unnatural amino acids or modified amino acids as a starting reagent. Examples of bioorthogonal conjugation reactions include but are not limited to, Staudinger ligation, copper-catalyzed azide-alkyne cycloaddition, strain promoted [3+2] cycloadditions, tetrazine ligation, metal-catalyzed coupling reactions, or oxime-hydrazone ligations. Examples of non-natural amino acids include, but are not limited to, azidohomoalanine, 2-homopropargylglycine, 3-homoallylglycine, 4-p-acetyl-Phe, 5-p-azido-Phe, 3-(6-acetylnaphthalen-2-ylamino)-2-aminopropanoic acid, N^{ε}-(cyclooct-2-yn-1-yloxy)carbonyl)L-lysine, N^{ε}-2-azideoethyloxycarbonyl-L-lysine, N∈-p-azidobenzyloxycarbonyl lysine, propargyl-L-lysine, or trans-cyclooct-2-ene lysine.

In some embodiments, the linker is derived from a small molecule, such as a polymer. Example polymer linkers include but are not limited to, poly-ethylene glycol, poly(N-isopropylacrylamide), and N,N′-dimethylacrylamide)-co-4-phenylazophenyl acrylate. The small molecule linkers generally include one or more reactive handles allowing conjugation to the second protein, the flap nuclease, or both. In some embodiments, the reactive handle allows for a bioconjugation or bioorthogonal conjugation. In some embodiments, the reactive handle allows for any organic reaction compatible with conjugating a linker to the second protein, the flap nuclease, or both.

The linker may be conjugated at any amino acid location of the second protein, the flap nuclease, or both. For example, the linker may be conjugated to the N-terminus, C-terminus, or any amino acid between of the flap nuclease, the second protein, or both. In some embodiments, the linker is conjugated to the N terminus of the flap nuclease and the N terminus of the second protein. In some embodiments, the linker is conjugated to the C terminus of the flap nuclease and the C terminus of the second protein. In some embodiments, the linker is conjugated to the C terminus of the flap nuclease and the N terminus of the second protein. In some embodiments, the linker is conjugated to the N terminus of the flap nuclease and the C terminus of the second protein.

In embodiments, where the flap nuclease and the second protein are operably linked by a peptide linker, the flap nuclease-second protein construct may be referred to as a fusion construct. Stated differently in some embodiments, a flap nuclease-second protein construct may be a fusion construct. Fusion constructs can be produced by expression in a host cell (e.g., recombinant expression).

In some embodiments, the second protein may be a polymerase. In some embodiments, the flap nuclease may be operably linked to a second protein that may be a polymerase. Any suitable polymerase may be used in a flap nuclease-polymerase construct. Examples of suitable polymerases may be found in US Patent Application Number U.S. Ser. No. 16/703,569 (U.S. Ser. No. 11/001,816B2), PCT Application Number PCT/US2013/03169 (WO2014142921A1), all of which are hereby incorporated by reference in their entireties. In some embodiments, the polymerase has strand displacing activity. In some embodiments, the polymerase does not have strand displacing activity.

In some embodiments, the flap nuclease-polymerase construct includes the Taq DNA polymerase or a portion thereof that has flap nuclease activity. In some embodiments, the flap nuclease-polymerase construct includes the Bst DNA polymerase or a portion thereof that has flap nuclease activity. In some embodiments, the flap nuclease-polymerase construct includes FEN1 or a portion thereof that has flap nuclease activity. In some embodiments, the flap nuclease-polymerase construct includes GAN or a portion thereof that has flap nuclease activity. In some embodiments, the flap nuclease-polymerase includes the Bst DNA polymerase or a portion thereof that has flap nuclease activity, and the linker includes the sequence of SEQ ID NO. 5, SEQ ID NO. 6, SEQ ID NO. 7, or SEQ ID NO. 10. In some embodiments, the flap nuclease-polymerase includes the Taq DNA polymerase or a portion thereof that has flap nuclease activity, and the linker includes the sequence of SEQ ID NO. 5, SEQ ID NO. 6, SEQ ID NO. 7, or SEQ ID NO. 10. In some embodiments, the flap nuclease-polymerase includes FEN1 or a portion thereof that has flap nuclease activity, and the linker includes the sequence of SEQ ID NO. 5, SEQ ID NO. 6, SEQ ID NO. 7, or SEQ ID NO. 10. In some embodiments, the flap nuclease-polymerase includes GAN or a portion thereof that has flap nuclease activity, and the linker includes the sequence of SEQ ID NO. 5, SEQ ID NO. 6, SEQ ID NO. 7, or SEQ ID NO.10.

In some embodiments, the second protein of a flap nuclease-second protein construct may be a DNA binding domain. In some embodiments, the flap nuclease of the construct may be operably linked to a second protein that may be a DNA binding domain. Any suitable DNA binding protein or domain may be used in a flap nuclease-DNA binding domain construct. A DNA binding domain is a protein or a portion of a protein that has an affinity for DNA, RNA, or both. In some embodiments, the DNA binding domain is a dsDNA binding domain. The terms “dsDNA binding domain” and “dsDNA binding protein,” as used herein refer generally to a protein or a domain of a protein that has an affinity to double stranded DNA. In some embodiments, the DNA binding domain is a sequence-nonspecific DNA binding domain. The terms “sequence-nonspecific DNA binding protein” and “sequence-nonspecific DNA binding domain” as used herein refer generally to DNA binding domains that do not have any preference for specific nucleotide sequences. A sequence-nonspecific dsDNA binding domain fused to a flap nuclease may stabilize the association between the flap nuclease and the nicked oligonucleotide strand (e.g., GG from FIG. 5A), thereby increasing the flap nuclease's efficacy in preventing the formation of and/or removing an impeding strand flap of strand (e.g., strand GG) during sequencing.

Examples of DNA binding domains and proteins include transcription factors, DNA repair proteins, and other types of proteins having an affinity for DNA, RNA, or both. A DNA binding domain may be from any source, such as, for example, eukaryotes, archaea, prokaryotes, or fungi. A DNA binding domain may be synthetic or derived from a natural DNA binding domain. For example, DNA binding domain from a natural source can be mutated to form a DNA binding domain derived from the natural source. The mutated DNA binding domain may have increased DNA binding affinity, decreased DNA binding affinity, increased DNA sequence specificity, decreased DNA sequence specificity, or any combination thereof. Table 2 gives examples of DNA binding proteins. Although a specific source is shown for DNA binding proteins in Table 2, the same or similar DNA binding protein may be isolated from a different source. In some embodiments, the DNA binding protein is a DNA binding protein from Table 2, or a portion thereof that has DNA binding ability.

TABLE 2

| DNA | | | |
|---|---|---|---|
| Binding | Type of binding | Source of | |
| domain | domain | domain | Reference |
| dsRNA | RNA binding | RNase III, | Cher Ling Tong, Nisha Kanwar, |
| binder | domain | Escherichia coli | Dana J Morrone, Burckhard Seelig, |
| | | | Nature-inspired engineering of an |
| | | | artificial ligase enzyme by domain |
| | | | fusion, Nucleic Acids Research, |
| | | | Volume 50, Issue 19, 28 Oct. |
| | | | 2022, Pages 11175-11185, |
| | | | doi.org/10.1093/nar/gkac858 |
| Zinc | RNA binding | NC, Human | Cher Ling Tong, Nisha Kanwar, |
| knuckle | domain | immunodeficiency | Dana J Morrone, Burckhard Seelig, |
| | | virus 1 | Nature-inspired engineering of an |
| | | | artificial ligase enzyme by domain |
| | | | fusion, Nucleic Acids Research, |
| | | | Volume 50, Issue 19, 28 Oct. |
| | | | 2022, Pages 11175-11185, |
| | | | doi.org/10.1093/nar/gkac858 |
| Helix, | RNA binding | NC, Human | Cher Ling Tong, Nisha Kanwar, |
| Zinc | domain | immunodeficiency | Dana J Morrone, Burckhard Seelig, |
| knuckle | | virus 1 | Nature-inspired engineering of an |
| | | | artificial ligase enzyme by domain |
| | | | fusion, Nucleic Acids Research, |
| | | | Volume 50, Issue 19, 28 Oct. |
| | | | 2022, Pages 11175-11185, |
| | | | doi.org/10.1093/nar/gkac858 |
| Helix- | DNA binding | Engrailed, | Cher Ling Tong, Nisha Kanwar, |
| turn- | domain | Drosophila | Dana J Morrone, Burckhard Seelig, |
| helix | | melanogaster | Nature-inspired engineering of an |
| | | | artificial ligase enzyme by domain |
| | | | fusion, Nucleic Acids Research, |
| | | | Volume 50, Issue 19, 28 Oct. |
| | | | 2022, Pages 11175-11185, |
| | | | doi.org/10.1093/nar/gkac858 |
| Zinc | DNA binding | TFIIIA, Xenopus | Cher Ling Tong, Nisha Kanwar, |
| finger | domain | laevis | Dana J Morrone, Burckhard Seelig, |
| | | | Nature-inspired engineering of an |
| | | | artificial ligase enzyme by domain |
| | | | fusion, Nucleic Acids Research, |
| | | | Volume 50, Issue 19, 28 Oct. |
| | | | 2022, Pages 11175-11185, |
| | | | doi.org/10.1093/nar/gkac858 |
| Di-RGG | Arginine-rich | hnRNP K, Homo | Cher Ling Tong, Nisha Kanwar, |
| box | peptides | sapiens | Dana J Morrone, Burckhard Seelig, |
| | | | Nature-inspired engineering of an |
| | | | artificial ligase enzyme by domain |
| | | | fusion, Nucleic Acids Research, |
| | | | Volume 50, Issue 19, 28 Oct. |
| | | | 2022, Pages 11175-11185, |
| | | | doi.org/10.1093/nar/gkac858 |
| Tri- | Arginine-rich | hnRNP U, Homo | Cher Ling Tong, Nisha Kanwar, |
| RGG | peptides | sapiens | Dana J Morrone, Burckhard Seelig, |
| box | | | Nature-inspired engineering of an |
| | | | artificial ligase enzyme by domain |
| | | | fusion, Nucleic Acids Research, |
| | | | Volume 50, Issue 19, 28 Oct. |
| | | | 2022, Pages 11175-11185, |
| | | | doi.org/10.1093/nar/gkac858 |
| NF-kB | Eukaryotic | Homo sapiens | Robert H. Wilson, Susan K. Morton, |
| p50 | transcription | | Heather Deiderick, Monica L. Gerth, |
| | factors | | Hayden A. Paul, Ilana Gerber, |
| | | | Ankita Patel, Andrew D. Ellington, |
| | | | Scott P. Hunicke-Smith, Wayne M. |
| | | | Patrick, Engineered DNA ligases |
| | | | with improved activities in |
| | | | vitro, Protein Engineering, Design |
| | | | and Selection, Volume 26, Issue 7, |
| | | | July 2013, Pages 471- |
| | | | 478, doi.org/10.1093/protein/gzt024 |
| NFAT | Eukaryotic | Mus musculus | Robert H. Wilson, Susan K. Morton, |
| | transcription | | Heather Deiderick, Monica L. Gerth, |
| | factors | | Hayden A. Paul, Ilana Gerber, |
| | | | Ankita Patel, Andrew D. Ellington, |
| | | | Scott P. Hunicke-Smith, Wayne M. |
| | | | Patrick, Engineered DNA ligases |
| | | | with improved activities in |
| | | | vitro, Protein Engineering, Design |
| | | | and Selection, Volume 26, Issue 7, |
| | | | July 2013, Pages 471- |
| | | | 478, doi.org/10.1093/protein/gzt024 |
| cTF | Eukaryotic | M. musculus/ | Robert H. Wilson, Susan K. Morton, |
| | transcription | H. sapiens | Heather Deiderick, Monica L, Gerth, |
| | factors | | Hayden A. Paul, Ilana Gerber, |
| | | | Ankita Patel, Andrew D. Ellington, |
| | | | Scott P. Hunicke-Smith, Wayne M. |
| | | | Patrick, Engineered DNA ligases |
| | | | with improved activities in |
| | | | vitro, Protein Engineering, Design |
| | | | and Selection, Volume 26, Issue 7, |
| | | | July 2013, Pages 471- |
| | | | 478, doi.org/10.1093/protein/gzt024 |
| PprA | Bacterial DNA | Synthetic | Robert H. Wilson, Susan K. Morton, |
| | repair proteins | | Heather Deiderick, Monica L. Gerth, |
| | | | Hayden A. Paul, Ilana Gerber, |
| | | | Ankita Patel, Andrew D. Ellington, |
| | | | Scott P. Hunicke-Smith, Wayne M. |
| | | | Patrick, Engineered DNA ligases |
| | | | with improved activities in |
| | | | vitro, Protein Engineering, Design |
| | | | and Selection, Volume 26, Issue 7, |
| | | | July 2013, Pages 471- |
| | | | 478, doi.org/10.1093/protein/gzt024 |
| Ku | Bacterial DNA | Strain H37Rv, | Robert H. Wilson, Susan K. Morton, |
| | repair proteins | genomic DNA | Heather Deiderick, Monica L. Gerth, |
| | | | Hayden A. Paul, Ilana Gerber, |
| | | | Ankita Patel, Andrew D. Ellington, |
| | | | Scott P. Hunicke-Smith, Wayne M. |
| | | | Patrick, Engineered DNA ligases |
| | | | with improved activities in |
| | | | vitro, Protein Engineering, Design |
| | | | and Selection, Volume 26, Issue 7, |
| | | | July 2013, Pages 471- |
| | | | 478, doi.org/10.1093/protein/gzt024 |
| [(HhH)2]2 | Archaeal DNA- | Methanopyrus | Robert H. Wilson, Susan K. Morton, |
| | binding domains | kandleri | Heather Deiderick, Monica L. Gerth, |
| | | | Hayden A. Paul, Ilana Gerber, |
| | | | Ankita Patel, Andrew D. Ellington, |
| | | | Scott P. Hunicke-Smith, Wayne M. |
| | | | Patrick, Engineered DNA ligases |
| | | | with improved activities in |
| | | | vitro, Protein Engineering, Design |
| | | | and Selection, Volume 26, Issue 7, |
| | | | July 2013, Pages 471- |
| | | | 478, doi.org/10.1093/protein/gzt024 |
| DNA | DNA-binding | Pyrococcus | Spibida M, Krawczyk B, Zalewska- |
| Ligase | domain | furiosus | Piatek B, Piatek R, Wysocka M, |
| | | | Olszewski M. Fusion of DNA- |
| | | | binding domain of Pyrococcus |
| | | | furiosus ligase with TaqStoffel DNA |
| | | | polymerase as a useful tool in PCR |
| | | | with difficult targets. Appl |
| | | | Microbiol Biotechnol. 2018 |
| | | | January; 102(2):713-721. doi: |
| | | | 10.1007/s00253-017-8560-6. Epub |
| | | | 2017 Nov. 4. PMID: 29103168; |
| | | | PMCID: PMC5756566. |
| DNA | DNA-Binding | Pyrococcus | Spibida M, Krawczyk B, Zalewska- |
| Ligase | Domain | abyssi | Piatek B, Piatek R, Wysocka M, |
| | | | Olszewski M. Fusion of DNA- |
| | | | binding domain of Pyrococcus |
| | | | furiosus ligase with TaqStoffel DNA |
| | | | polymerase as a useful tool in PCR |
| | | | with difficult targets. Appl |
| | | | Microbiol Biotechnol. 2018 |
| | | | January;102(2):713-721. doi: |
| | | | 10.1007/s00253-017-8560-6. Epub |
| | | | 2017 Nov. 4, PMID: 29103168; |
| | | | PMCID: PMC5756566. |
| NeqSSB- | DNA-Binding | Nanoarchaeum | Spibida M, Krawczyk B, Zalewska- |
| like | Domain | equitans | Piatek B, Piatek R, Wysocka M, |
| | | | Olszewski M. Fusion of DNA- |
| | | | binding domain of Pyrococcus |
| | | | furiosus ligase with TaqStoffel DNA |
| | | | polymerase as a useful tool in PCR |
| | | | with difficult targets. Appl |
| | | | Microbiol Biotechnol. 2018 |
| | | | January; 102(2):713-721. doi: |
| | | | 10.1007/s00253-017-8560-6. Epub |
| | | | 2017 Nov. 4. PMID: 29103168; |
| | | | PMCID: PMC5756566. |

In some embodiments, the DNA binding domain includes Sso7d, or a portion thereof having affinity for DNA. Sso7d is a sequence-nonspecific dsDNA binding domain that is commercially available (Aviva Systems Biology; Cusabio; Biorbyt). In some embodiments, Sso7d can be encoded by SEQ ID NO. 10 (ATGGCAACCGTCAAGTTTAAATACAAAGGCGAGGAGAAGGAGGTGGACATC AGCAAAATCAAAAAAGTATGGCGTGTCGGGAAAATGATTTCGTTTACCTACG ACGAGGGCGGGGGGAAGACCGGACGTGGAGCAGTATCAGAAAAGGATGCCC CGAAAGAACTTTTGCAAATGCTTGAAAAACAGAAAAAG) or a portion thereof. When fused to a polymerase, Sso7d may stabilize the polymerase's association to the nucleotide template, resulting in a greater processivity, as expressed by a greater number of nucleotides incorporated per binding of the polymerase to the template. Other examples of DNA binding proteins include HMG1/2-like protein; HMG-D; Sac7d; FK506-binding protein 25 (FKBP25); mycobacterium DNA binding protein Dps1; and MukB

In some embodiments, the flap nuclease-DNA binding domain construct includes a flap nuclease and a DNA binding domain linked through a linker of sequence SEQ ID NO. 5, SEQ ID NO. 6, SEQ ID NO. 7, or SEQ ID NO.10. In some embodiments, the flap nuclease-polymerase construct includes a flap nuclease and Sso7D linked through a linker of sequence SEQ ID NO. 5, SEQ ID NO. 6, SEQ ID NO. 7, or SEQ ID NO.10. In some embodiments, the flap nuclease-polymerase construct includes GAN and Sso7D linked through a linker of the sequence SEQ ID NO. 5, SEQ ID NO. 6, SEQ ID NO. 7, or SEQ ID NO.10. In some embodiments, the flap nuclease-polymerase construct includes GAN and Sso7D linked through a linker of sequence SEQ ID NO.10.

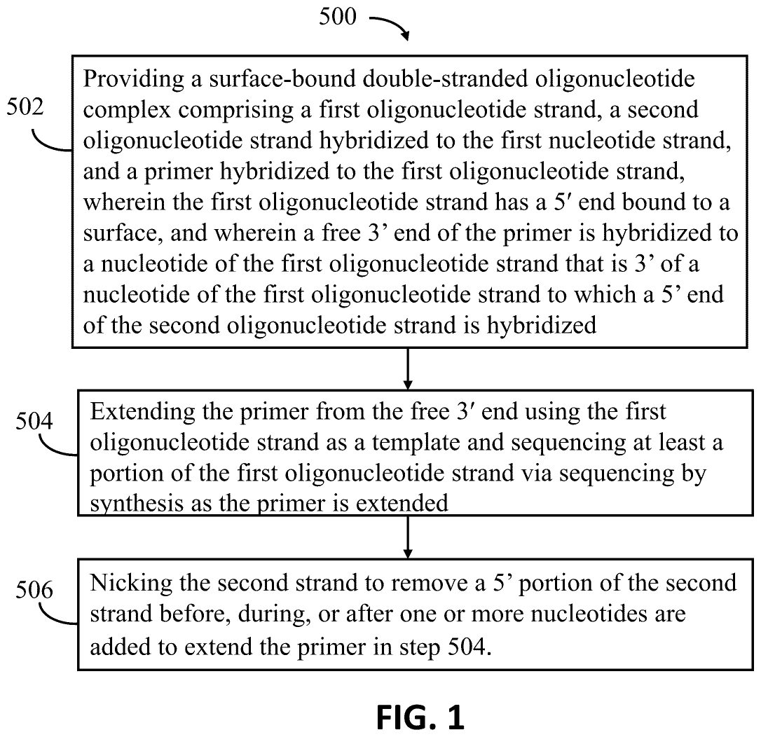

FIGS. 1, 2, 3, 4, 5B, 6A, 6B, 6C, 7A, 7B, 8A, and 8B are referenced to illustrate embodiments consistent with the present disclosure. For clarity, the description of each element and step in the figures is described in the singular. However, it should be understood that the sequencing and pre-sequencing methods described herein may be applied to arrays or cluster of polynucleotides provided as described previously in order to accomplish massively parallel sequencing.

FIG. 1 is a flow chart illustrating an overview of a double-stranded sequencing method using nick translation (500) consistent with some embodiments of the present disclosure. The method includes providing a surface-bound double-stranded oligonucleotide complex (502). The double-stranded oligonucleotide complex includes a first oligonucleotide strand, a second oligonucleotide strand hybridized to at least a portion of the first oligonucleotide strand, and a primer hybridized to the first nucleotide strand. The first oligonucleotide strand has a 5′ end bound to a surface. The primer has a free 3′end. In some embodiments, the primer has a free 5′ end. The free 3′ end of the primer is hybridized to a nucleotide of the first oligonucleotide strand this is 3′ of a nucleotide of the first oligonucleotide strand to which a 5′ end of the second oligonucleotide strand is hybridized. The method includes extending the primer from the free 3′ end using the first oligonucleotide as a template and sequencing at least a portion of the first oligonucleotide strand via sequencing by synthesis as the primer is extended (504). The method further includes nicking the second oligonucleotide strand to remove a 5′ portion of the second oligonucleotide strand before, during, or after one or more nucleotides are added to the extended primer in step 504 (506). In some embodiments, the second strand is nicked in step 506 before, during, or after each nucleotide is added to extend the primer in step 504. In other embodiments, the second oligonucleotide strand is nicked in step 506 after more than one nucleotide is added to extend the primer in step 504.

The sequencing by synthesis and nick translation method 500 may be used and is consistent with other sequencing methods/workflows described herein.

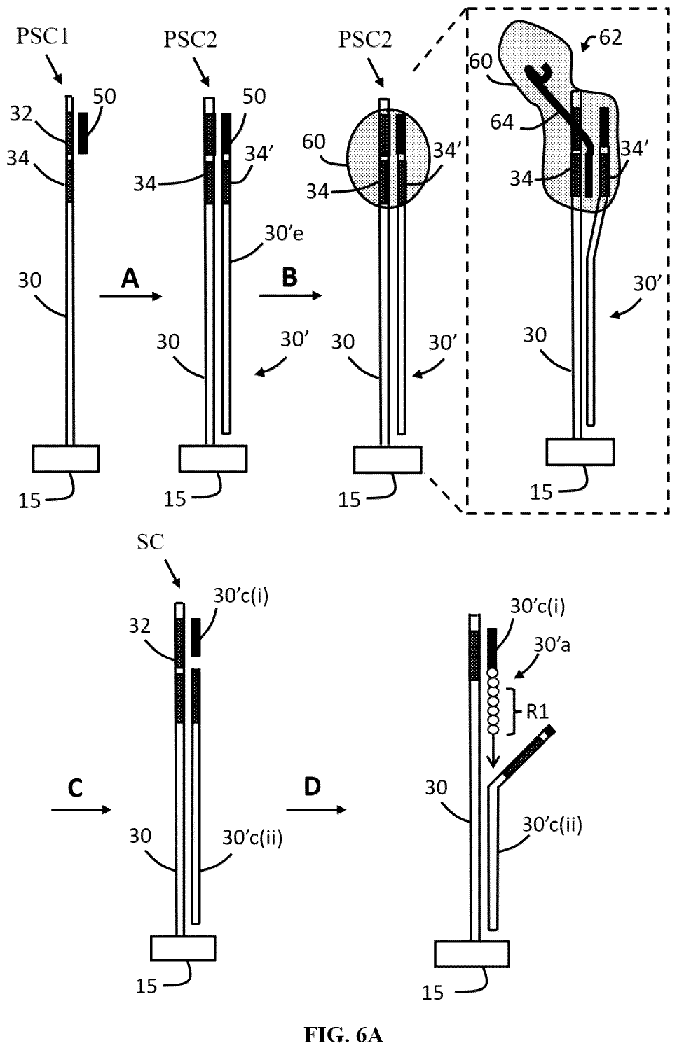

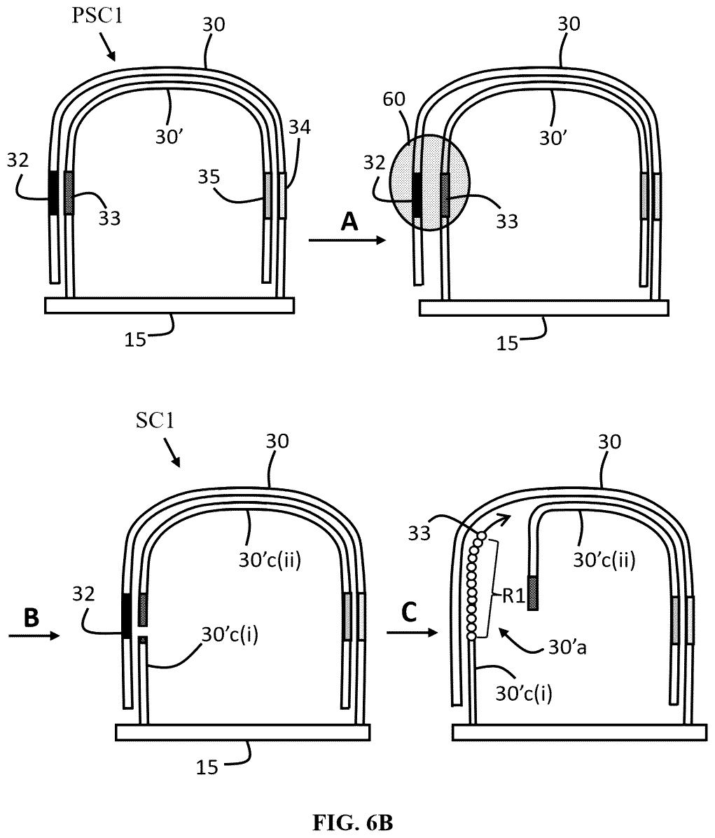

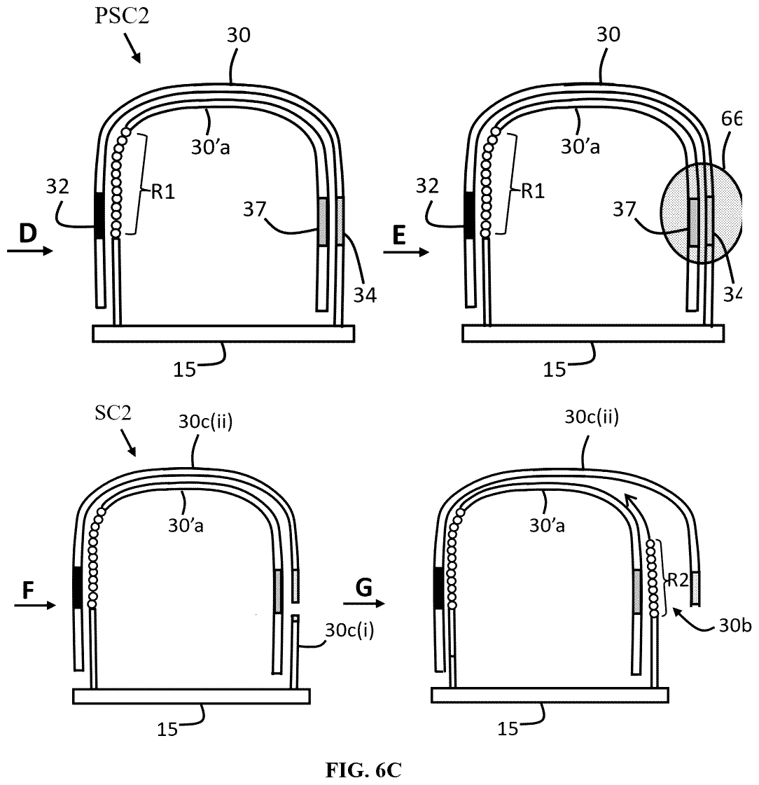

FIG. 5B provides a schematic overview of a double-stranded surface sequencing method consistent with some embodiments of the present disclosure, for example, the double-stranded sequencing method via nick translation 500 of FIG. 1. The workflow of FIG. 5A includes providing a sequencing complex SC. Sequencing complex SC includes a first oligonucleotide strand 30 having a 5′ end bound to a surface 15. The first oligonucleotide strand 30 includes an extension primer binding region 92. In some embodiments where the first oligonucleotide strand 30 includes adapters, the adapter sequence proximate the 5′ end of the first oligonucleotide strand 30 includes the primer binding region 92. In some embodiments, the primer binding region 92 is proximate the 3′ end of the first oligonucleotide strand 30. The SC also includes a primer 90 hybridized to at least a portion of the primer binding region 92 of the first oligonucleotide strand 30. The primer is a sequencing primer; that is, the primer is used as a primer for sequencing at least a portion of the first oligonucleotide strand 30. The primer 90 has a free 3′ end. In some embodiments, the primer 90 has a free 5′ end (as shown in FIG. 5B). In other embodiments, the primer has a 5′ end that is bound to the surface. For example, in some sequencing methods described later herein (e.g., method 200), the sequencing primer is formed through the cleaved of an oligonucleotide strand bound to the surface and the 5′ end of the sequencing primer is therefore bound to the surface (e.g., see 30′c(i) in FIG. 6B). The terminal nucleotide on the 3′ end of the primer 90 is hybridized to nucleotide of the first oligonucleotide strand 30 that is 3′ to the nucleotide of the first oligonucleotide strand 30 that the terminal 5′ nucleotide of the second oligonucleotide 30′ strand is hybridized to. Stated differently, from the perspective of the first oligonucleotide strand 30, the primer 90 is hybridized 3′ to the second oligonucleotide strand 30′.

In step A of FIG. 5B, at least a portion of the first oligonucleotide strand 30 is sequenced as a first read region R1 (e.g., step 504 of method 500). Sequencing may include sequencing by synthesis where the 3′ end of the primer 90 serves as a sequencing primer. As such, the primer 90 is enzymatically extended in the 5′ to 3′ direction by adding nucleotides DD thereby creating a portion of a third oligonucleotide strand 30′a that is complementary to and hybridized to the first oligonucleotide strand 30. In some embodiments, the third oligonucleotide strand 30′a has a 5′ end that is immobilized to the surface (e.g., when the primer has a 5′ end that is immobilized on the surface. In other embodiments, the third oligonucleotide strand 3-′a has a free 5′ end (e.g., when the primer has a free 5′ end). The portion of the third oligonucleotide strand 30′a generated during sequencing is the first read region R1. The enzymatic extension uses the first oligonucleotide strand 30 as the template and thee primer 90 sequencing primer. Before, during, or after one or more nucleotides DD are added to primer 90 or the third oligonucleotide strand 30′a the second oligonucleotide stand is nicked to removed one or more 5′ nucleotides of the second oligonucleotide strand 30′ impeding the growth of the third oligonucleotide strand 30′a via nick translation.

Any flap nuclease of flap nuclease operably linked to a polymerase, such as those described herein, may be used to nick and/or enzymatically introduce new nucleotides to the primer or third oligonucleotide strand 30′a. In some embodiments, the second oligonucleotide strand 30′ is nicked before, during, or after each nucleotide is added to extend the primer 90 or third oligonucleotide strand 30′a. In such embodiments, the flap nuclease or flap nuclease operably linked to a polymerase is present at each nucleotide incorporation step. In other embodiments, the second oligonucleotide strand is nicked after more than one nucleotide is added to extend the primer 90 or third oligonucleotide strand 30′a. In such embodiments, a flap nuclease or flap nuclease operably linked to a polymerase may be added after one or more nucleotides are added to extend the primer 90 or third oligonucleotide strand 30′a; that is, a flap is allowed to form.

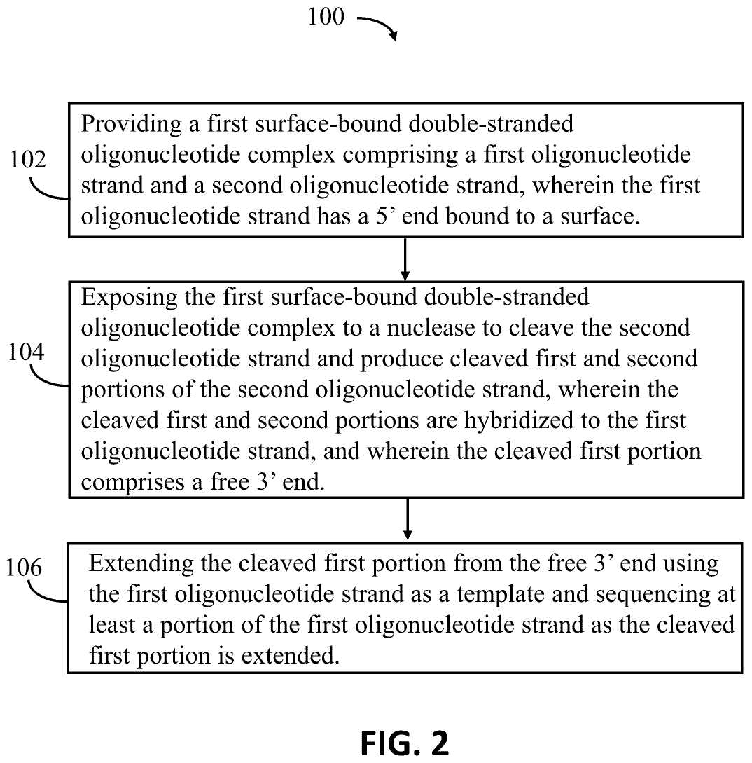

FIG. 2 is a flow chart illustrating an overview of a first double-stranded sequencing method (100) consistent with some embodiments of the present disclosure. The method includes providing a surface-bound double-stranded oligonucleotide complex (102). The surface-bound double-stranded complex includes a first oligonucleotide strand having a 5′ end bound to the surface and a second oligonucleotide strand. The method further includes exposing the first surface bound double-stranded oligonucleotide complex to a nuclease to cleave the second oligonucleotide strand producing a cleaved first portion and a cleaved second portion of the second oligonucleotide strand (104). The cleaved first portion and cleaved second portions of the second oligonucleotide strand are hybridized to the first oligonucleotide strand. The cleaved first portion of the second oligonucleotide strand includes a free 3′ end. The method further includes extending the cleaved first portion of the second oligonucleotide strand from the free 3′ end using the first oligonucleotide strand as a template and sequencing at least a portion of the first oligonucleotide strand as the cleaved first portion of the second oligonucleotide strand is extended (106).

In some embodiments of method 100, in step 102, the second oligonucleotide strand has a 5′ end bound to the surface. In some such embodiments, in step 104, the cleaved first portion of the second oligonucleotide strand includes a 5′ end bound to the surface. Extending the cleaved first portion of the second oligonucleotide strand generates a second surface-bound double-stranded oligonucleotide complex that includes the first oligonucleotide strand and the nascent third oligonucleotide strand both bound to the surface.

In some embodiments of method 100, the method further includes exposing the second surface bound double-stranded oligonucleotide complex to a nuclease to cleave the first oligonucleotide strand and produce a cleaved third portion and a cleaved fourth portion of the first oligonucleotide strand. The cleaved third portion and cleaved fourth portion of the first oligonucleotide strand are hybridized to the third oligonucleotide strand. For clarity regarding other cleaved portions of oligonucleotides of method 100, the cleaved portions of the first oligonucleotide strand are referred to as third and fourth portions to distinguish from the cleaved first and cleaved second portions of the second oligonucleotide strand but can be thought of as cleaved first and cleaved second portions of the first oligonucleotide strand. The cleaved third portion of the first oligonucleotide includes a surface bound 5′ end and a free 3′ end. In some embodiments, the method further includes extending the cleaved third portion of the first oligonucleotide strand from the free 3′ end using the third oligonucleotide strand as a template and sequencing at least a portion of the third oligonucleotide strand as the cleaved third portion of the first oligonucleotide strand is extended.

FIGS. 6A-6C provide a schematic overview of a double-stranded surface sequencing method consistent with some embodiments of the present disclosure, for example, the double-stranded sequencing method 100 of FIG. 2. The workflow of FIG. 6A includes providing a pre-sequencing complex PSC1 (an optional step of method 100). Pre-sequencing complex PSC1 includes a first oligonucleotide strand 30 having a free 3′ end and a 5′ end bound to a surface 15. The first oligonucleotide strand 30 includes an extension primer binding region 32. In some embodiments, the first oligonucleotide strand includes a nuclease binding region 34. The extension primer binding region 32 and the nuclease binding region 34 may be located at any location on the first oligonucleotide strand 30. The extension primer binding region 32 and the nuclease binding region 34 may be the same, different, or have overlapping portions. In some embodiments where the first oligonucleotide strand 30 includes adapters, the adapter sequence proximate the 3′ end of the first oligonucleotide strand 30 includes the extension primer binding region 32, the nuclease binding region 34, or both. The PSC1 also includes an extension primer 50 hybridized to at least a portion of the extension primer binding region 32 of the first oligonucleotide strand 30. The extension primer 50 has a free 3′ end and a free 5′ end.

In step A of FIG. 6A, the extension primer 50 is enzymatically extended from the free 3′ end using the first oligonucleotide strand 30 as a template to create as second oligonucleotide strand 30′ and a second pre-sequencing complex PSC2. The second pre-sequencing complex PSC2 includes the first oligonucleotide strand 30 hybridized to the second oligonucleotide strand 30′. The second oligonucleotide strand 30′ has a free 3′ end, a free 5′ end, and includes the extension primer 50 and an extended portion 30′e. In some embodiments, the second oligonucleotide strand 30′ includes a nuclease binding region 34′. Pre-sequencing complex PSC2 is an example of the surface bound double-stranded oligonucleotide complex of step 102 of method 100 wherein the second oligonucleotide strand is not bound to the surface.

In step B of FIG. 6A, the pre-sequencing complex (i.e., surface bound double-stranded oligonucleotide complex) is exposed to nuclease 60 (e.g., step 104 of method 100). A nuclease is a protein that is capable of cleaving the phosphodiester backbone between two adjacent nucleotides. The nuclease recognizes and binds to the nuclease binding region 34 of the first oligonucleotide strand 30 or the nuclease binding region 34′ of the second oligonucleotide strand 30′.

In step C of FIG. 6A, the nuclease 60, cleaves the second oligonucleotide strand 30′ into a cleaved first portion 30′c(i) and a cleaved second portion 30′c(ii) to produce the sequencing complex SC (e.g., step 104 of method 100). The sequencing complex SC includes the first oligonucleotide strand 30 hybridized to the cleaved first portion 30′c(i) and cleaved second portion 30′c(ii) of the second oligonucleotide strand 30′. The cleaved first portion 30′c(i) and the cleaved second portion 30′c(ii) each have a free 3′ end and a free 5′ end.

In step D of FIG. 6A, at least a portion of the first oligonucleotide strand 30 is sequenced as a first read region R1. Sequencing may include sequencing by synthesis where the 3′ end of the cleaved first portion 30′c(i) of the second oligonucleotide strand 30′ serves as a sequencing primer. As such, the cleaved first portion 30′c(i) of the second oligonucleotide strand 30′ is enzymatically extended in the 5′ to 3′ direction thereby creating a portion of a third oligonucleotide strand 30′a that is complementary to the first oligonucleotide strand 30. The portion of the third oligonucleotide strand 30′a generated during sequencing is the first read region R1. The enzymatic extension uses the first oligonucleotide strand 30 as the template and the cleaved first portion 30′c(i) of the second oligonucleotide strand 30′ as the sequencing primer. In some embodiments, the double-stranded sequencing may proceed by strand displacement, nick translation, or a combination thereof.

Step B of FIG. 6A includes exposing the pre-sequencing complex to a nuclease. Any suitable nuclease may be used. In some embodiments, the nuclease is a restriction endonuclease having single strand nicking activity. In some embodiments, the nuclease comprises a ribonucleoprotein having a ribonucleic acid component that guides a protein component having single strand nicking activity to a nuclease binding region to nick a strand at a predefined location. In some embodiments, the pre-sequencing complex is exposed to a CRISPR-Cas nuclease, a ZINC finger nuclease, or a transcription activator-like effector nuclease (TALEN).

In some embodiments, a nuclease cleaves a phosphodiester backbone between two adjacent nucleotides such that a first oligonucleotide strand including the 3′ nucleotide of the adjacent nucleotides having a free 3′ hydroxyl and a second oligonucleotide strand including the 5′ nucleotide of the adjacent nucleotides having a 5′ phosphate are formed. In some embodiments, a nuclease cleaves a phosphodiester backbone between two adjacent nucleotides such that a first oligonucleotide strand including the 3′ nucleotide of the adjacent nucleotides having a free 3′ phosphate and a second oligonucleotide strand including the 5′ nucleotide of the adjacent nucleotides having a 5′ hydroxyl are formed. A nuclease may be employed to cleave the phosphodiester backbone between two adjacent nucleotides of an oligonucleotide strand to produce a first oligonucleotide strand (e.g., a cleaved first portion 30′c(i)) having a free 3′ hydroxyl or a free 3′ phosphate and a second oligonucleotide strand (e.g., a cleaved second portion 30′c(ii)) having a free 5′ phosphate or a free 5′ phosphate at the location of the cleavage. In embodiments where the nuclease produces a first oligonucleotide strand having a free 3′ phosphate, the phosphate group can be removed to expose a 3′ hydroxyl suitable for chain extension using techniques disclosed herein.

In some embodiments, the nuclease is a Cas nuclease from a CRISPR-Cas gene editing system. In such embodiments, the nuclease is complexed with a guide RNA to form a ribonucleoprotein (RNP). The RNP is able to selectively cleave a phosphodiester backbone between two adjacent nucleotides.

In some embodiments, the first double-stranded sequencing method (100) employs a ribonucleoprotein (RNP) to cleave the second oligonucleotide strand 30′ at a specific location to produce a cleaved first portion 30′c(i) having a free 3′ end that is used as a sequencing primer. An RNP is a complex between one or more ribonucleic acids and a ribonucleic acid binding protein. The ribonucleic acid binding protein may have single strand nicking activity.

In some embodiments, the nucleases of the present disclosure are a Cas nuclease of CRISPR-Cas gene editing systems or a variant thereof. A nuclease variant may be a nuclease that is truncated; fused to another protein such as another nuclease; includes one or more mutations that increase binding affinity, decrease binding affinity, increase cleavage efficacy, decrease cleavage efficacy, remove cleavage ability; or any combination thereof. In embodiments, a nuclease variant may have at least 80%, at least 85%, at least 90%, at least 95%, at least 96%, at least 97%, at least 98%, at least 99%, or 100% sequence identity to the nuclease from which it was derived. An active fragment of a nuclease is a nuclease variant that includes a functional cleavage domain.

The Cas nuclease may be a Class 1 Cas nuclease or a Class 2 Cas nuclease or a variant thereof. In nature, Class 1 Cas nucleases use a complex of multiple Cas proteins to degrade foreign nucleic acids and Class 2 Cas nucleases using a single Cas protein to degrade foreign nucleic acids. Class 1 Cas nucleases include Type 1, Type 3, and Type 4. Class 2 Cas nuclease include Type 2, Type 5, and Type 6. Table 3 shows example Cas proteins and their respective Class and Type. In some embodiments, the nucleases of the present disclosure are or are a variant of any one of the nucleases in Table 3.

TABLE 3

| Cas Protein | Class | Type | Cas Protein | Class | Type |
|---|---|---|---|---|---|
| Cas3 | 1 | 1 | Cas4 | 2 | 2 |
| Cas8a | 1 | 1 | Cas12 | 2 | 5 |
| Cas8b | 1 | 1 | Cas12a (Cpf1) | 2 | 5 |
| Cas8c | 1 | 1 | Cas12b (C2c1) | 2 | 5 |
| Cas10d | 1 | 1 | Cas12c (C2c3) | 2 | 5 |
| Cse1 | 1 | 1 | Cas12d (CasY) | 2 | 5 |
| Cse2 | 1 | 1 | Cas12e (CasX) | 2 | 5 |
| Cys1 | 1 | 1 | Cas12f (Cas14, C2c10) | 2 | 5 |
| Csy2 | 1 | 1 | Cas12g | 2 | 5 |
| Cys3 | 1 | 1 | Cas12h | 2 | 5 |
| GSU0054 | 1 | 1 | Cas12i | 2 | 5 |
| Cas10 | 1 | 3 | Cas12k (C2c5) | 2 | 5 |
| Csm2 | 1 | 3 | C2c4 | 2 | 5 |
| Cmr5 | 1 | 3 | C2c8 | 2 | 5 |
| Cas10 | 1 | 3 | C2c9 | 2 | 5 |
| Csx11 | 1 | 3 | Cas13 | 2 | 6 |
| Csx10 | 1 | 3 | Cas13a (C2c2) | 2 | 6 |
| Csf1 | 1 | 4 | Cas13b | 2 | 6 |
| Cas9 | 2 | 2 | Cas13c | 2 | 6 |
| Csn2 | 2 | 2 | Cas13d | 2 | 6 |

The Cas protein can be a Cas protein from any organism. For example, a Cas protein can be or derived from a Cas protein isolated from Streptococcus pyogenes, Staphylococcus aureus, Neisseria meningitidis, Francisella novicida, Campylobacter jejuni, Lachnospiraceae bacterium, Acidaminococcus sp., Streptococcus canis, or Staphylococcus auricularis.

The Cas protein can be chosen based on the protospacer adjacent motif (PAM) recognized by the Cas protein. A PAM is a sequence of oligonucleotides recognized by the Cas protein often downstream of the cleavage location. Different Cas proteins can have different PAMs. Examples of Cas proteins and the PAMs they recognize are shown in Table 4.

TABLE 4

| PAM sequence (5′ | | |
|---|---|---|
| to 3′) | Nuclease | Isolated (I) of Derived (D) from |
| NGG | SpCas9 | (I) Streptococcus pyogenes |
| NGRRT or NGRRN | SaCas9 | (I) Staphylococcus aureus |
| NNNNGATT | NmeCas9 | (I) Neisseria meningitidis |
| NNNNRYAC | CjCas9 | (I) Campylobacter jejuni |
| NNAGAAW | StCas9 | (I) Streptococcus thermophiles |
| TTTV | LbCpf1 | (I) Lachnospiraceae bacterium |
| TTTV | AsCpf1 | (I) Acidaminococcus sp. |
| NGAG | SpCAs9-VQR | (D) Streptococcus pyogenes |
| NGCG | SpCas9-VRER | (D) Streptococcus pyogenes |
| NGN | SpCas9-NG | (D) Streptococcus pyogenes |
| NG, GAA, GAT | SpCas9-xCas9 | (D) Streptococcus pyogenes |
| NNG | SpCas9-Sc++ | (D) Streptococcus pyogenes |
| NGN | SpCas9-SpG | (D) Streptococcus pyogenes |
| N(G/A)N | SpCas9-SpRY | (D) Streptococcus pyogenes |
| NGG | FnCas9 | (I) Francisella novicida |
| (C/T)G | FnCas9-RHA | (D) Francisella novicida |
| NNN(G/A)(G/A)T | SaCas9-KKH | (D) Staphylococcus aureus |
| NNAGAA(A/T) | St1Cas9 | (I) Streptococcus thermophilus |
| NNNNC(G/A)AA | GeoCas9 | (I) Geobacillus stearothermophilus |
| T(C/T)C(A/C/G) | AsCas 12a-RR | (D) Acidaminococcus sp. |
| TAT(A/C/G) | AsCas 12-RVR | (D) Acidaminococcus sp. |
| TTT(A/C/G) | FnCas12a | (I) Francisella novicida |
| TTN | Cas12j | phage |
| TTCN | Cas12e | |
| TTTN | Un1Cas12f1 | Uncultured archaeon |
| CCN | CnCas12f1 | |

In some embodiments, the Cas protein of the present disclosure is or is a variant of a Cas protein isolated from Streptococcus pyogenes. In some such embodiments, the Cas protein is SpCas9, SpCas9-HF, eSpCas9, SpCas9-VQR, SpCas9-VRER, SpCas9-NG, SpCas9-xCas9, SpCas9-Sc++, SpCas9-SpG, SpCas9-SpRY, or a variant thereof.

In some cases, Cas proteins include two nucleases or two nuclease domains, with one nuclease or domain configured to nick one strand of a double-stranded oligonucleotide and the other nuclease or domain configured to nick the other strand of a double-stranded oligonucleotide. In some embodiments, one of the two nucleases or domains is mutated such that cleaving activity of the mutated nuclease is prevented, providing one active nuclease or domain that will nick only a single strand. For example, Cas9 includes two nucleases, RuvC and HNH each of which cleave a different strand of a double-stranded complex. In some embodiments where Cas9 is the nuclease, the RuvC domain is deactivated. In other embodiments, the NHN is deactivated.

RNPs may include an oligonucleotide component that guides the nuclease to a sequence-specific location of a double stranded oligonucleotide. The oligonucleotide component may be referred to as a “guide RNA”, or “gRNA.” The gRNA includes a sequence that is at least partially complementary to a portion of the first oligonucleotide strand or the second oligonucleotide strand (e.g., a complementary to a nuclease binding region of the first or second strand). Depending on the nuclease or variant thereof used in connection with the gRNA, the gRNA may nick the strand to which the gRNA is hybridized or the strand opposite to which the gRNA is hybridized.

The gRNA also includes a scaffold sequence that has an affinity for the Cas protein allowing RNP formation. Suitable scaffold sequences for use with various Cas proteins and variants are well known in the art.

The length and composition of the gRNA binding sequence can vary depending on the desired location of the cleavage. In some embodiments, the gRNA binding sequence is at least partially complementary to an adapter sequence. The gRNA binding sequence can include any modified nucleobases, modified sugars, and/or modified internucleoside backbone linkages that still allow for hybridization to the first oligonucleotide strand or the second oligonucleotide strand. For example, the gRNA binding sequence may include one or more modifications to increase the stability of the gRNA. In some embodiments, the gRNA binding sequence is 5 to 50, 5 to 40, 5 to 30, 5 to 25, 5 to 20, 5 to 15, 5 to 10, 10 to 30, 10 to 25, 15 to 25, or 15 to 25 nucleotides in length.

FIG. 6A shows an example of an RNP 62 binding to the second pre-sequencing complex PSC2. The RNP includes a gRNA 64 and a nuclease 60. The gRNA 64 includes a binding sequence that is at least partially complementary to the nuclease binding region 34 of the first oligonucleotide strand 30. A portion of the second oligonucleotide strand 30′ is displaced from being hybridized to the first oligonucleotide strand 30 upon the RNP binding the first oligonucleotide strand 30. The nuclease 60 cleaves the second oligonucleotide strand 30′ giving a cleaved first portion 30′c(i) and a cleaved second portion 30′c(ii). A Cas nuclease able to cleave the strand not involved in hybridization to the gRNA may be used.

Although not depicted in FIG. 6A, the gRNA can include a binding sequence that is at least partially complementary to the nuclease binding region 34′ on the second oligonucleotide strand 30′. In this case, a portion of the first oligonucleotide strand 30 is displaced from being hybridized to the second oligonucleotide strand 30 upon the RNP binding the second oligonucleotide strand 30′. The nuclease 60 cleaves the second oligonucleotide strand 30′ giving a cleaved first portion 30′c(i) and a cleaved second portion 30′c(ii). A Cas nuclease able to cleave the strand hybridized to the gRNA may be used.

In some embodiments, the nuclease 60 is a zinc finger nuclease. The term “zinc finger nuclease,” refers to a nuclease comprising a nucleic acid cleavage domain conjugated to a binding domain that includes a zinc finger array. A zinc finger is a nucleic acid-binding protein structural motif characterized by a fold and the coordination of one or more zinc ions that stabilize the fold. Zinc fingers can be designed to bind a specific sequence of nucleotides. Two or more zinc fingers can be fused to make a zinc finger array. Such arrays can be designed to bind to any desired sequence. Zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs.

In some embodiments, the nuclease 60 is a TALEN. The term “Transcriptional Activator-Like Element Nuclease,” (TALEN) refers to an artificial nuclease comprising a transcriptional activator like (TAL) effector DNA binding domain and a DNA cleavage domain, for example, a FokI domain. TALENS can be engineered to bind to and cleave at specific sequences. A number of modular assembly schemes for generating engineered TALEN constructs have been reported.

In some embodiments of the first double-stranded sequencing method (100), both the first oligonucleotide strand and the second oligonucleotide strand have 5′ ends that are bound to the surface and free 3′ ends. FIG. 6B shows the workflow of such a method. The workflow includes providing a pre-sequencing complex PSC1 (e.g., step 102 of method 100). The first pre-sequencing complex PSC1 includes a first oligonucleotide strand 30 having a free 3′ end and a 5′ end bound to a surface 15. In some embodiments, the first oligonucleotide strand 30 includes a first nuclease binding region 32 and/or a second nuclease binding region 34. The first pre-sequencing complex PSC1 includes a second oligonucleotide strand 30′ having a free 3′ end and a 5′ end bound to the surface 15. In some embodiments, the second oligonucleotide strand 30′ includes a third nuclease binding region 33 and/or a fourth nuclease binding region 35. The first oligonucleotide strand 30 and the second oligonucleotide strand 30′ are at least partially complementary and are hybridized in a double-stranded bridged structure. The first pre-sequencing complex PCS1 may be provided upon the completion of bridge amplification (described herein).

In step A of FIG. 6B, the first pre-sequencing complex PSC1 (i.e., surface bound double-stranded oligonucleotide complex) is exposed to a nuclease 60 (e.g., step 104 of method 100). The nuclease may be any nuclease described herein. The nuclease binds to either the first nuclease binding region 32 of the first oligonucleotide strand 30 or the third nuclease binding region 33 of the second oligonucleotide strand 30′.

In step B of FIG. 6B, the nuclease 60, cleaves the second oligonucleotide strand 30′ into a cleaved first portion 30′c(i) and a cleaved second portion 30′c(ii) to produce a first sequencing complex SC1 (e.g., step 104 of method 100). The first sequencing complex SC1 includes the first oligonucleotide strand 30 hybridized to the cleaved first portion 30′c(i) and cleaved second portion 30′c(ii) of the second oligonucleotide strand 30′. The cleaved first portion 30′c(i) has a free 3′ end and a 5′ end bound to the surface 15. The cleaved second portion 30′c(ii) has a free 3′ end and a free 5′ end.

In step C of FIG. 6B, at least a portion of the first oligonucleotide strand 30 is sequenced as a first read region R1 (e.g., step 106 of method 100). Sequencing may include sequencing by synthesis where the 3′ end of the cleaved first portion 30′c(i) of the second oligonucleotide strand 30′ serves as a sequencing primer. As such, the cleaved first portion 30′c(i) of the second oligonucleotide strand 30′ is enzymatically extended in the 5′ to 3′ direction thereby creating a portion of a third oligonucleotide strand 30′a that is complementary to and hybridized to the first oligonucleotide strand 30 and has a 5′ end that is surface bound. The portion of the third oligonucleotide strand 30a generated during sequencing is the first read region R1. The enzymatic extension uses the first oligonucleotide strand 30 as the template and the cleaved first portion 30′c(i) of the second oligonucleotide strand 30′ as the sequencing primer. In some embodiments, the double-stranded sequencing may proceed via strand displacement or nick translation.

As depicted in FIG. 6C, the steps of FIG. 6B can be repeated after the formation of a second pre-sequencing complex PSC2 to sequence at least a portion of the third oligonucleotide strand 30′a. In step D of FIG. 6C, the third oligonucleotide strand 30′a is extended from the first read region R1 through the incorporation of nucleotides using the first oligonucleotide strand 30 as a template creating a second presequencing complex PSC2. The nucleotides incorporated in step D may be blocked nucleotides, such as those used in SBS, or unblock nucleotides allowing for rapid chain extension. In the second pre-sequencing complex PSC2, the third oligonucleotide strand 30′a is covalently bonded to the surface 15 at its 5′ end and has a free 3′ end. In some embodiments, the third oligonucleotide strand 30′a includes a fifth nuclease binding region 37. Additionally, the third oligonucleotide strand 30′a is complementary to the first oligonucleotide strand 30a and includes the first read region R1 proximate its 5′ end. The first oligonucleotide strand 30a and the third oligonucleotide strand 30′a are at least partially hybridized in a double-stranded bridged structure.

In step E of FIG. 6C, the second pre-sequencing complex PSC2 is exposed to a second nuclease 66. The nuclease may be any nuclease described herein. In some embodiments, the second nuclease 66 is the same as the first nuclease 60. In other embodiments, the second nuclease 66 is different than the first nuclease 60. The second nuclease 66 recognizes and binds to the second nuclease binding region 34 of the first oligonucleotide strand 30 or the fifth nuclease binding region 37 of the third oligonucleotide strand 30′a.

In step F of FIG. 6C, the second nuclease 66, cleaves the first oligonucleotide strand 30 into a cleaved first portion 30c(i) and a cleaved second portion 30c(ii) to produce a second sequencing complex SC2. The second sequencing complex SC2 includes the third oligonucleotide strand 30′a hybridized to the cleaved first portion 30c(i) and cleaved second portion 30c(ii) of the first oligonucleotide strand 30. The cleaved first portion 30c(i) has a free 3′ end and a 5′ end bound to the surface 15. The cleaved second portion 30c(ii) has a free 3′ end and a free 5′ end.

In step G of FIG. 6C, at least a portion of the third oligonucleotide strand 30′a is sequenced as a second read region R2. Sequencing may include sequencing by synthesis where the 3′ end of the cleaved first portion 30c(i) of the first oligonucleotide strand 30 serves as a sequencing primer. As such, the cleaved first portion 30c(i) of the first oligonucleotide strand 30′ is enzymatically extended in the 5′ to 3′ direction thereby creating a portion of a fourth oligonucleotide strand 30b that is complementary to and hybridized to the third oligonucleotide strand 30′a and has a 5′ end that is surface bound. The portion of the third oligonucleotide strand 30b generated during sequencing is the second read region R2. The enzymatic extension uses the third oligonucleotide strand 30′a as the template and the cleaved first portion 30c(i) of the first oligonucleotide strand 30 as the sequencing primer.

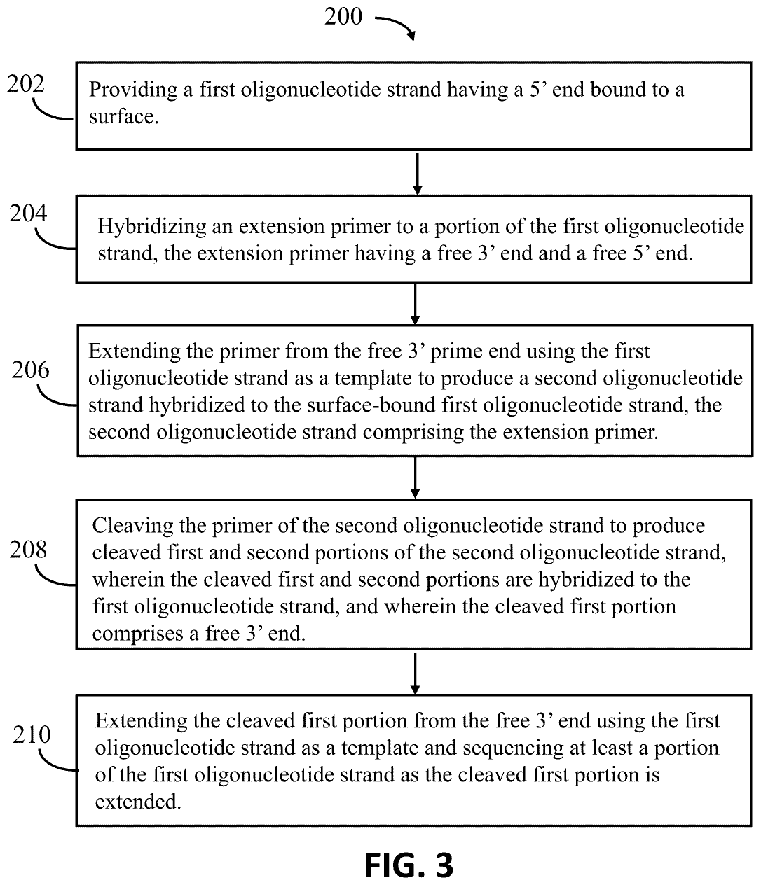

FIG. 3 is a flow chart illustrating an overview of a second double-stranded sequencing method (200) consistent with some embodiments of the present disclosure. The second method includes providing a first oligonucleotide strand having a 5′ end bound to a surface (202). The method further includes hybridizing an extension primer to a portion of the first oligonucleotide strand, the primer having a free 5′ end and a free 3′ end (204). The method further includes extending the primer from the free 3′ end using the first oligonucleotide strand as a template to produce a second oligonucleotide strand hybridized to the surface-bound first oligonucleotide strand (206). The second oligonucleotide strand includes the extension primer. The method further includes cleaving the extension primer of the second oligonucleotide strand to produce a cleaved first portion and a cleaved second portion of the second oligonucleotide strand (208). The cleave first portion and the cleaved second portion are hybridized to the first oligonucleotide strand. Additionally, the cleaved first portion includes a free 3′ end. The method further includes extending the cleaved first portion from the free 3′ end using the first oligonucleotide strand as a template and sequencing at least a portion of the first oligonucleotide strand as the cleaved first portion is extended (210).

In some embodiments of the second double-stranded sequencing method 200, step 202 further comprises providing a surface oligonucleotide having a free 3′ end and having a 5′ end bound to the surface. In such embodiments, the first oligonucleotide strand includes a 3′ portion that is at least partially complementary to at least a portion of the surface oligonucleotide. As such, the 3′ portion of the first oligonucleotide strand is hybridized to the surface oligonucleotide forming a single stranded bridge structure. In step 206, the single stranded bridged structure is converted into a double-stranded bridged structure.

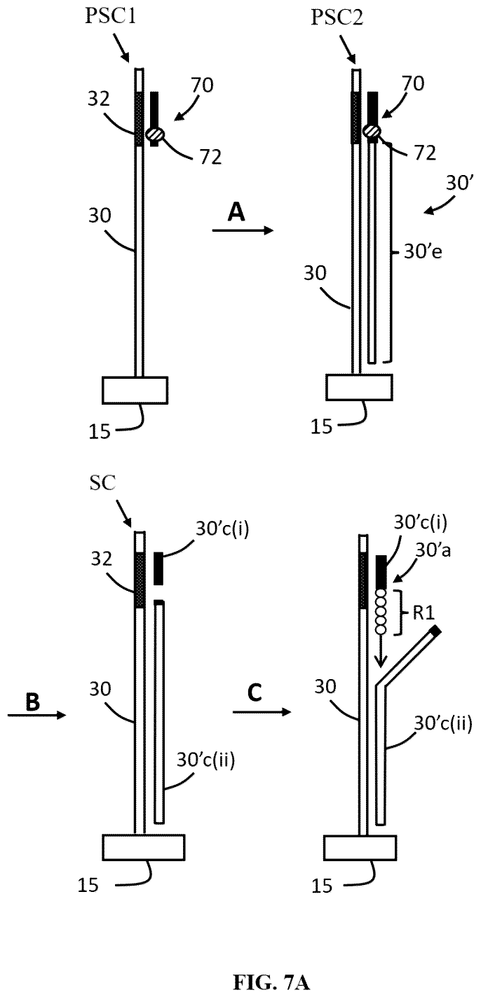

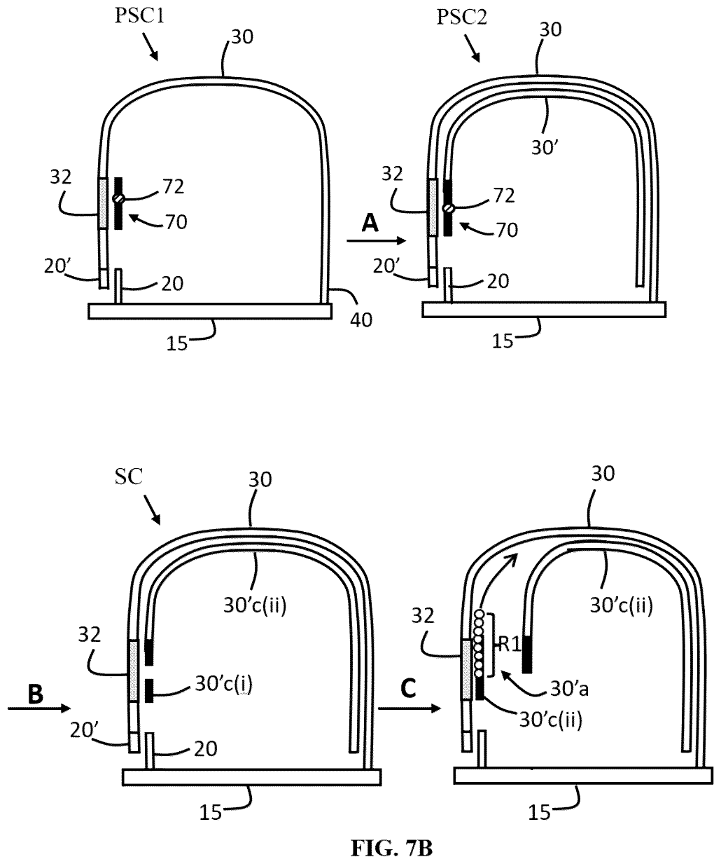

FIGS. 7A and 7B provide a schematic overview of a single strand surface sequencing method consistent with some embodiments of the present disclosure, for example, the second double-stranded sequencing method of FIG. 3. The workflow of FIG. 7A includes providing a pre-sequencing complex PSC1. Pre-sequencing complex PSC1 includes a first oligonucleotide strand 30 having a free 3′ end and a 5′ end bound to a surface 15. The first oligonucleotide strand 30 includes an extension primer binding region 32. The extension primer binding region 32 may be located at any location on the first oligonucleotide strand 30. In some embodiments where the first oligonucleotide strand 30 includes adapters, the adapter sequence proximate the 3′ end of the first oligonucleotide strand 30 includes the extension primer binding region 32. The first pre-sequencing complex PSC1 also includes an extension primer 70 hybridized to at least a portion of the extension primer binding region 32 of the first oligonucleotide strand 30. The extension primer 70 has a free 3′ end and a free 5′ end and includes at least one cleavage site 72.

In step A of FIG. 7A, the extension primer 70 is enzymatically extended from the free 3′ end using the first oligonucleotide strand 30 as a template to create a second oligonucleotide strand 30′ and a second pre-sequencing complex PSC2. The second pre-sequencing complex PSC2 includes the first oligonucleotide strand 30 hybridized to the second oligonucleotide strand 30′. The second oligonucleotide strand 30′ has a free 3′ end, a free 5′ end, includes the extension primer 70, and includes extended region 30′e.

In step B of FIG. 7A, the extension primer 70 of the second oligonucleotide strand 30′ is cleaved at the cleavage site 72 creating a sequencing complex SC. The sequencing complex SC includes a cleaved first portion 30′c(i) and a cleaved second portion 30′c(ii) of the second oligonucleotide strand 30′. The cleaved first portion 30′c(i) includes a free 5′ end, a free 3′ end having a 3′ hydroxyl, and at least a portion of the extension primer 70. The cleaved second portion 30′c(ii) includes a free 5′ end and a free 3′ end. In some embodiments, the cleaved second portion 30′c(ii) includes a portion of the extension primer 70 at its free 5′ end. The sequencing complex also includes the cleaved first portion 30′c(i) and cleaved second portion 30′c(ii) of the second oligonucleotide strand 30′ hybridized to the first oligonucleotide strand 30 in a double-stranded structure.

In step C of FIG. 7A, at least a portion of the first oligonucleotide strand 30 is sequenced as a first read region R1. Sequencing may include sequencing by synthesis where the free 3′ end of the cleaved first portion 30′c(i) of the second oligonucleotide strand 30′ serves as a sequencing primer. As such, the cleaved first portion 30′c(i) of the second oligonucleotide strand 30′ is enzymatically extended in the 5′ to 3′ direction thereby creating a portion of a third oligonucleotide strand 30′a that is complementary to and hybridized to the first oligonucleotide strand 30. The portion of the third oligonucleotide strand 30′a generated during sequencing is the first read region R1. The enzymatic extension uses the first oligonucleotide strand 30 as the template and the cleaved first portion 30′c(i) of the second oligonucleotide strand 30′ as the sequencing primer. The double-stranded sequencing may occur via strand displacement or nick translation.

In step B of FIG. 7A, the extension primer 70 of the second oligonucleotides strand 30′ is cleaved to give a cleaved first portion 30′c(i) and a cleaved second portion 30′c(ii) of the second oligonucleotide strand 30′. Various cleavage methods may be used including, for example, abasic cleavage, chemical cleavage, cleavage of ribonucleotides, photochemical cleavage, hemimethylated DNA cleavage, nicking endonuclease cleavage, and restriction enzyme cleavage, some of which are described in more detail below.

### Abasic Cleavage

In some embodiments, abasic cleavage is used to cleave the extension primer 70 of the second oligonucleotide strand 30′. In some embodiments, the extension primer 70 of the second oligonucleotide strand 30′ includes an excisable base at the cleavage location 72. The excisable base is generally configured to be removed from the second oligonucleotide strand 30′. The excisable base may be located anywhere along the extension primer 70.

In some embodiments, the excisable base is removed from the extension primer 70 of the second oligonucleotide strand 30′ resulting in an abasic site. An “abasic site” is a nucleotide position in an oligonucleotide strand from which the base component of the nucleotide has been removed. Abasic sites can be formed chemically under artificial conditions or by the action of enzymes.

In some embodiments, an abasic site may be created at a pre-determined position on the primer 70 of the second oligonucleotide strand 30′. This can be achieved, by incorporating a specific excisable base at the pre-determined position.

The excisable base may be any base or modified base that can be removed from a double-stranded DNA. Example excisable bases include, but are not limited to, deoxyuridine (dU); 8-oxo-guanine (8-oxo-G); deoxyinosine; 7,8-dihydro-8-oxoguanine (8-oxoguanine); 8-oxoadenine; fapy-guanine; methyl-fapy-guanine; fapy-adenine; aflatoxin B1-fapy-guanine; 5-hydroxy-cytosine; 5-hydroxy-uracil; and the like. In some embodiments, deoxyuridine may be provided by heat assisted deamination of 5-methyl cytosine (methyl-C), bisulfite assisted deamination of methyl-C, or both. Enzymes that may be used to create an abasic site include, but are not limited to, uracil DNA glycosylase (UDG); an uracil specific excision reagent enzyme such as USER (available from New England BioLabs located in Ipswich, MA); FPG glycosylase; AlkA glycosylase; oxoguanine glycosylase, and the like. In some embodiments, the excisable base is deoxyuridine (dU). In some such embodiments, UDG and/or an uracil specific excision reagent enzyme is used to create the abasic site. In some embodiments, the excisable base is 8-oxo-G. In some such embodiments, FPG glycosylase is used to create an abasic site.

Once formed, an abasic site may be cleaved providing a means for site-specific cleavage of an oligonucleotide strand. For example, removal of the abasic site generated after the removal of the excisable base in the extension primer 70 of the second oligonucleotide strand 30′, will generate the cleaved first portion 30′c(i) and cleaved second portion 30′c(ii) of the second oligonucleotide strand 30′. The oligonucleotide strand that includes the abasic site can then be cleaved at the abasic site by treatment with an endonuclease such as DNA glycosylase-lyase endonuclease VIII; AP lyase; FPG glycosylase; heat; or alkali conditions to yield a 3′ phosphate on the 3′ terminal end on one of the portions of the cleaved oligonucleotide (e.g., the cleaved first portion 30′c(i)). In some embodiments, a mixture containing the appropriate glycosylase and one or more suitable endonucleases, typically in an activity ratio of at least about 2:1, is used to generate the abasic site and cleave the oligonucleotide strand at the abasic site in a single step. For example, in some embodiments, the surface is treated with a mixture of uracil DNA glycosylase and endonuclease VII to generate an abasic site at the excisable base and cleave the oligonucleotide strand having the excisable base to generate two oligonucleotide strands one of which having a terminal 3′ phosphate on the free 3′ end. In reference to FIG. 6A, the surface is treated with a mixture of uracil DNA glycosylates and endonuclease VII to generate an abasic site at the excisable base of the primer region 70 of the second oligonucleotide strand 30′ and cleave the second oligonucleotide strand 30′ at the abasic site to generate a cleaved first portion 30′c(i) having a terminal 3′ phosphate on the free 3′ end.

For enzymatic oligonucleotide strand extension, the terminal 3′ nucleotide of an oligonucleotide strand is required to have a hydroxyl at the 3′ position of the deoxyribose. In embodiments where abasic cleavage results in a 3′ terminal nucleotide having a 3′ terminal phosphate, additional treatments can be done to remove the phosphate producing a 3′ hydroxyl. Examples of enzymes that can be used to generate a hydroxyl from a phosphate include, but are not limited to, T4 polynucleotide kinase (T4PNK), endonuclease IV, and suitable phosphatases such as calf intestinal phosphatase, shrimp alkaline phosphatase, and Pyrococcus abysii alkaline phosphatase.

Advantages of the abasic cleavage method may include the option of releasing a free 3′ phosphate group on the cleaved strand, which after treatment to generate terminal 3′ hydroxyl group can provide an initiation point for sequencing. Because the cleavage reaction requires a residue, e.g., deoxyuridine, which does not occur naturally in DNA, but is otherwise independent of sequence context, if only one non-natural base is included there is no possibility of glycosylase-mediated cleavage occurring elsewhere at unwanted positions in the double-stranded structure. An advantage gained by cleavage of abasic sites in a double-stranded section of an immobilized polynucleotide templates generated by action of UDG on uracil is that the first base incorporated in a sequencing-by-synthesis reaction initiating at the free 3′ hydroxyl group formed by cleavage will always be T. As a result, for all clonal clusters at different amplification sites of an array which are cleaved in this manner to produce sequencing templates the first base universally incorporated across the whole array will be T. This can provide a sequence-independent assay for individual cluster intensity at the start of a sequencing run.

In some embodiments, the abasic cleavage of an excisable base and the generation of the hydroxyl at the 3′ position of the deoxyribose of the free 3′ end of a cleaved oligonucleotide strand (e.g., the cleaved first portion 30′c(i)) may be accomplished in one step. In some embodiments, a single reagent may be used to excise the excisable base of the primer region 70 of the second oligonucleotide strand 30′ and generate a terminal hydroxyl at the 3′ position on the deoxyribose of the free 3′ end of the cleaved first portion 30′c(i). For example, EndoQ from Pyrococcus furious (Pfu) recognizes and cuts the 5′ phosphodiester bond of uracil to generate a hydroxyl at the 3′ position of the deoxyribose on the nucleotide that is on the 5′ side of uracil (Ishino et al., Sci Rep. 2016 May 6; 6:25532. Doi: 10.1038/srep25532; Ishino et al., Nucleic Acids Res. 2015 Mar. 11; 43(5):2853-63. Doi: 10.1093/nar/gkv121). Thus, EndoQ may be used to both cleave the excisable base of the primer region 70 of the second oligonucleotide strand 30′ and generate a hydroxyl at the 3′ position of the deoxyribose of the free 3′ end of the cleaved first portion 30′c(i) of the second oligonucleotide strand 30′.

### Chemical Cleavage

In some embodiments, chemical cleavage methods are used to cleave at cleavage sites within an oligonucleotide to create two cleaved strands from the original uncleaved oligonucleotide strand. The term “chemical cleavage” encompasses any method which uses a non-enzymatic chemical reagent in order to promote/achieve cleavage of an oligonucleotide strand. If required, the oligonucleotide strand to be cleaved may include one or more non-nucleotide chemical moieties and/or non-natural nucleotides and/or non-natural backbone linkages, such as allyl-dNTPs, in order to permit a chemical cleavage reaction at the desired cleavage site.

In some embodiments, the oligonucleotide strand to be cleaved includes one or more ally-dNTPs such as, for example, allyl-T, allyl-A, allyl-G, or allyl-C at the desired cleavage site. The allyl-dNTP provides a site for chemical cleavage. In some embodiments, the allyl-dNTP allows for single step or two step cleavage and 3′ hydroxyl generation.

In some embodiments, an oligonucleotide strand including an allyl-dNTP at the desired cleavage site is cleaved and hydroxylated in two steps by treatment with Pd(0) and a hydroxyl forming reagent. For example, in reference to FIG. 7A, in some embodiments, the first step includes cleavage of cleavage site 72 within the primer region 70 of the second oligonucleotide strand 30′ with Pd(0) to produce a cleaved first portion 30′c(i) that has a free 3′ end that includes a terminal phosphate at the 3′ carbon of the deoxyribose sugar. In such embodiments, further treatment with one or more hydroxyl forming groups converts the phosphate into a terminal hydroxyl thereby generating a first cleave portion 30′c(i) that can be used as a primer (e.g., a sequencing primer). Examples of hydroxyl forming groups include, but are not limited to, T4 polynucleotide kinase (T4PNK), endonuclease IV, suitable phosphatases such as those described herein, or combinations thereof.

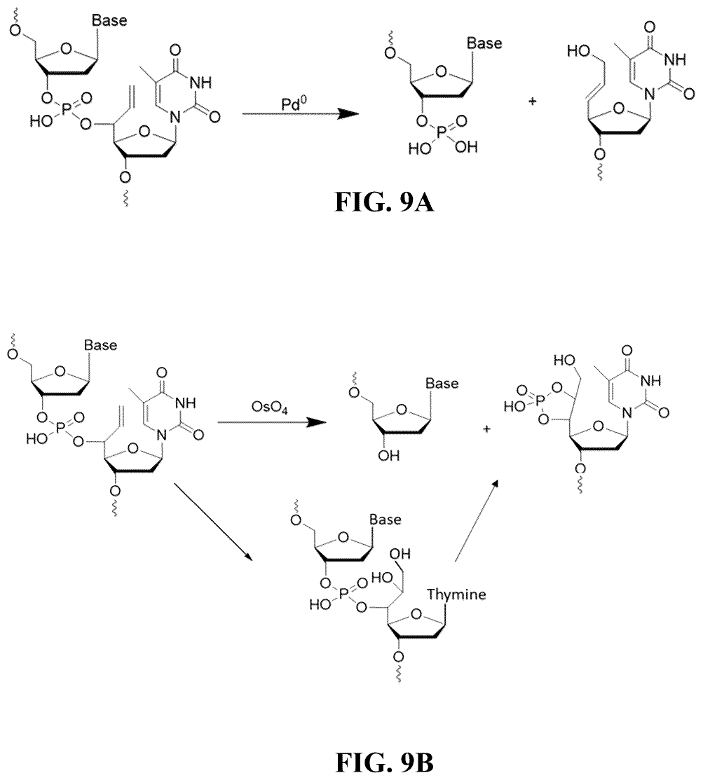

FIG. 9A shows an example cleavage reaction at the allyl-T cleavage site of an oligonucleotide strand using Pd(0). In FIG. 9A, the oligonucleotide (e.g., the extension primer region 70 of the second oligonucleotide strand 30′ in FIG. 7A) includes an allyl-T. Treatment with Pd(0) results in the cleavage of the oligonucleotide strand at the 5′ carbon of the deoxyribose sugar of allyl-T to produce two oligonucleotide strands. The 3′ end of the first oligonucleotide strand (e.g., the cleaved first portion 30′c(i)) has a phosphate group at the 3′ carbon of the terminal deoxyribose sugar. The 5′ end of the second oligonucleotide strand (e.g., the cleaved second portion 30′c(ii)) has an alkene and an alcohol extending from the 5′ carbon of the deoxyribose sugar of the allyl-T. The terminal phosphate at the 3′ carbon of the deoxyribose sugar of the first oligonucleotide can be further converted to a terminal hydroxyl through treatment with one or more hydroxyl forming reagents such as, for example, T4 polynucleotide kinase (T4PNK), endonuclease IV, or combinations thereof.

In some embodiments, an oligonucleotide that includes an allyl-dNTP at a cleavage site is cleaved to produce a 3′ hydroxyl in a single step via treatment with one or more reagents that dihydroxylate the alkene of the allyl-dNTP. In such embodiments, cleavage of an oligonucleotide strand to produce a first oligonucleotide strand having a free 3′ end that includes a terminal hydroxyl at the 3′ carbon of the deoxyribose includes treatment with one or more reagents that allow for a dihydroxylation reaction.

Dihydroxylation is the formation of a vicinal diol from an alkene. Without wishing to be bound by theory, it is thought that when an oligonucleotide containing an allyl-dNTP is subjected to a dihydroxylation reagent or reagents, the vicinal diol intermediate will decompose to form two oligonucleotides: a first oligonucleotide that includes a free 3′ end having a terminal hydroxyl on the 3′carbon of the terminal deoxyribose and a second oligonucleotide.

Any suitable dihydroxylation reagent or mixture of reagents may be used. Various alkene dihydroxylation reactions and the corresponding reagents are known, such as, for example, Sharpless asymmetric dihydroxylation, Milas dihydroxylation, Upjohn dihydroxylation, and Prevost and Woodward dihydroxylation. The Sharpless asymmetric dihydroxylation, Milas dihydroxylation, and Upjohn dihydroxylation use a catalyst and a stoichiometric oxidant to accomplish the dihydroxylation reaction. A common catalyst is osmium tetroxide (OsO₄). Stoichiometric oxidants include, but are not limited to, K₃[Fe(CN)₆], peroxide, water, and N-methylmorpholine N-oxide (NMO). The Prevost and Woodward dihydroxylation use iodine (I₂) and a silver salt (e.g., OHCO₂Ag) to accomplish dihydroxylation.

In some embodiments, cleavage of an oligonucleotide strand to form a cleaved first oligonucleotide strand (and a second cleaved oligonucleotide strand) that has a free 3′ end that includes a terminal hydroxyl at the 3′ carbon of the deoxyribose, includes treatment with a catalyst and a stoichiometric oxidant. In some embodiments, the catalyst is osmium tetroxide. In embodiments, the stoichiometric oxidant is K,[Fe(CN)A], peroxide, N-methylmorpholine N-oxide (NMO), water, or any combination thereof. In some embodiments, cleavage of an oligonucleotide strand to form a cleaved first oligonucleotide strand (and a second cleaved oligonucleotide strand) that has a free 3′ end that includes a terminal hydroxyl at the 3′ carbon of the deoxyribose, includes treatment with iodine and a silver salt.

In some embodiments, additional compounds, buffering agents, and/or solvents may be included in a dihydroxylation reaction. For example, various solvents may be included such as, water, t-butanol, isopropanol, or combinations thereof may be included in a dihydroxylation reaction.

In some embodiments in which OsO₄ is used, the OsO₄ may be formed in situ.

FIG. 9B shows an example cleavage reaction at the allyl-T cleavage site of an oligonucleotide strand using a dihydroxylation reaction. In FIG. 9B the oligonucleotide (e.g., the primer region 70 of the second oligonucleotide strand 30′) includes an allyl-T. Treatment with the dihydroxylation reagent, osmium tetroxide (OsO₄) and a stoichiometric oxidant results in the cleavage of the oligonucleotide strand at the 5′ carbon of the deoxyribose sugar of allyl-T to produce two oligonucleotide strands. The 3′ end of the first oligonucleotide strand (e.g., the cleaved first portion 30′c(i)) has a terminal hydroxyl group on 3′ carbon of the terminal deoxyribose sugar. Although the structure is unknown and not wishing to be bound by theory, the 5′ end of the second oligonucleotide strand (e.g., the cleaved second portion 30′c(ii)) is thought to have a five-member phosphate containing ring structure extending from the 5′ carbon of the terminal deoxyribose sugar of the allyl-T. The proposed five-member phosphate containing ring structure may decompose to form a different chemical group.

FIG. 9B also shows a proposed vicinal diol oligonucleotide intermediate structure that may occur post dihydroxylation but prior to separation of the oligonucleotide. Not wishing to be bound by theory, it is thought that the vicinal diol intermediate decomposes to give the first oligonucleotide and the second oligonucleotide having the terminal chemical groups as described above. In one embodiment, the surface oligonucleotides or polynucleotides include a diol linkage which permits cleavage by treatment with periodate (e.g., sodium periodate). It will be appreciated that more than one diol can be included at the cleavage site. Diol linker units based on phosphoramidite chemistry suitable for incorporation into a surface oligonucleotides or polynucleotides are commercially available from Fidelity systems Inc. (Gaithersburg, MD., USA). One or more diol units may be incorporated into oligonucleotides using standard methods for automated chemical DNA synthesis. Hence, the extension primers including one or more diol linkers can be conveniently prepared by chemical synthesis.

The diol linker is cleaved by treatment with a “cleaving agent,” which can be any substance that promotes cleavage of the diol. The preferred cleaving agent is periodate, such as aqueous sodium periodate (NaIO₄). Following treatment with the cleaving agent (e.g., periodate) to cleave the diol, the cleaved product may be treated with a “capping agent” in order to neutralize reactive species generated in the cleavage reaction. Suitable capping agents for this purpose include amines, such as ethanolamine. Advantageously, the capping agent (e.g., ethanolamine) can be included in a mixture with the cleaving agent (e.g., periodate) so that reactive species are capped as soon as they are formed. The resulting oligonucleotide may be treated to contain a 3′ hydroxyl group to enable use of the oligonucleotide as a primer for sequencing, chain extension, or sequencing and chain extension.

In another embodiment, the oligonucleotide strand to be cleaved can include a disulfide group which permits cleavage with a chemical reducing agent, e.g., tris(2-carboxyethyl)-phosphate hydrochloride (TCEP).

After chemical cleavage, one or more additional reagents, such as a phosphatase, may be needed to generate a terminal 3′ hydroxyl on a cleaved oligonucleotide strand generated from the cleavage (e.g., cleaved first portion 30′c(i) of the second oligonucleotide 30′).

### Cleavage of Ribonucleotides

Incorporation of one or more ribonucleotides into an oligonucleotide, which is otherwise made up of deoxyribonucleotides (with or without additional non-nucleotide chemical moieties, non-natural bases or non-natural backbone linkages) can provide a site for cleavage using a chemical agent capable of selectively cleaving the phosphodiester bond between a deoxyribonucleotide and a ribonucleotide or using a ribonuclease (RNAse). The oligonucleotide strand (e.g., the extension primer region 70 of the second oligonucleotide strand 30′) may be cleaved at a site containing one or more consecutive ribonucleotides using such a chemical cleavage agent or an RNase. In one embodiment, the strand to be cleaved contains a single ribonucleotide to provide a site for chemical cleavage.

Suitable chemical cleavage agents capable of selectively cleaving the phosphodiester bond between a deoxyribonucleotide and a ribonucleotide include metal ions, for example rare-earth metal ions (e.g., La³⁺, Tm³⁺, Yb³⁺, or Lu³⁺; Chen et al. Biotechniques. 2002, 32: 518-520; Komiyama et al. Chem. Commun. 1999, 1443-1451)), Fe(III) or Cu(III), or exposure to elevated pH (e.g., treatment with a base such as sodium hydroxide). By “selective cleavage of the phosphodiester bond between a deoxyribonucleotide and a ribonucleotide” is meant that the chemical cleavage agent is not capable of cleaving the phosphodiester bond between two deoxyribonucleotides under the same conditions.

The base composition of the ribonucleotide(s) is generally not material but can be selected in order to optimize chemical (or enzymatic) cleavage. By way of example, rUMP or rCMP are generally preferred if cleavage is to be carried out by exposure to metal ions, especially rare earth metal ions.

The phosphodiester bond between a ribonucleotide and a deoxyribonucleotide, or between two ribonucleotides may also be cleaved by an RNase. Any endocytic ribonuclease of appropriate substrate specificity can be used for this purpose. For cleavage with a ribonuclease it is preferred to include two or more consecutive ribonucleotides, such as from 2 to 10 or from 5 to 10 consecutive ribonucleotides. The precise sequence of the ribonucleotides is generally not material, except that certain RNases have specificity for cleavage after certain residues. Suitable RNases include, for example, RNaseA, which cleaves after C and U residues. Hence, when cleaving with RNaseA the cleavage site must include at least one ribonucleotide which is C or U.

Oligonucleotides incorporating one or more ribonucleotides can be readily synthesized using standard techniques for oligonucleotide chemical synthesis with appropriate ribonucleotide precursors.

After ribonuclease cleavage, one or more additional reagents, such as a phosphatase, may be needed to generate a terminal 3′ hydroxyl on a cleaved oligonucleotide strand.

### Photochemical Cleavage

The term “photochemical cleavage” encompasses any method which uses light energy in order to achieve cleavage of a nucleic acid. A site for photochemical cleavage can be provided by a non-nucleotide chemical spacer unit in an oligonucleotide strand. Suitable photochemical cleavable spacers include the PC spacer phosphoramidite (4-(4,4′-Dimethoxytrityloxy)butyramidomethyl)-1-(2-nitrophenyl)-ethyl]-2-cyanoethyl-(N,N-diisopropyl)-phosphoramidite) supplied by Glen Research, Sterling, Va., USA (cat number 10-4913-XX) which has the structure:

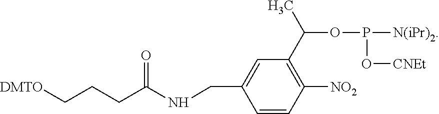

The spacer unit can be cleaved by exposure to a UV light source.

This spacer unit can be attached to the 5′ end of a polynucleotide, together with a thiophosphate group which permits attachment to a solid surface using standard techniques for chemical synthesis of oligonucleotides.

After photochemical cleavage, one or more additional reagents, such as a phosphatase, may be needed to generate a terminal 3′ hydroxyl on a cleaved oligonucleotide strand.

### Cleavage of Hemimethylated DNA

Site-specific cleavage of the surface oligonucleotide can also be achieved by incorporating one or more methylated nucleotides into an oligonucleotide, and then cleaving with an endonuclease enzyme specific for a recognition sequence including the methylated nucleotide(s).

The methylated nucleotide(s) will be opposite of non-methylated deoxyribonucleotides on the complementary strand, such that annealing of the two strands produces a hemimethylated duplex structure. The hemimethylated duplex may then be cleaved by the action of a suitable endonuclease.

Oligonucleotides incorporating one or more methylated nucleotides may be prepared using standard techniques for automated DNA synthesis, using appropriately methylated nucleotide precursors.

After cleavage of hemimethylated DNA, one or more additional reagents, such as a phosphatase, may be needed to generate a terminal 3′ hydroxyl.

### Nicking Endonuclease Cleavage

Nicking endonucleases are enzymes that selectively cleave or “nick” one strand of a double-stranded nucleic acid. Essentially any nicking endonuclease may be used, provided that a suitable recognition sequence can be included at the cleavage site present on the nucleic acid. Examples of nicking endonucleases include, but are not limited to, Nt.BspQI, Nt.CviPII, Nt.Btsl, and Nb.Bsml (all available from New England Biolabs, MA). Preferably, endonucleases that have long recognition sequences (e.g., 12-40 bp), such as homing endonucleases, are used as nicking endonuclease in order to prevent nonspecific nicking of an oligonucleotide strand. Homing endonucleases may be converted to nicking endonucleases for example, as described in Niu et al, (2008) JMB Vol 382: 188-20 and Molina et al., (2015) JBC Vol 290: 18534-18544. Examples of commercially available homing endonucleases that are nicking endonucleases include, but are not limited to, I-CeuI, I-SceI, PI-PspI, and PI-SceI (all available from New England Biolabs, MA).

After nicking endonuclease cleavage, one or more additional reagents, such as a phosphatase, may be needed to generate a terminal 3′ hydroxyl on the cleaved oligonucleotide strand.

In some embodiments of the second double-stranded sequencing method (200), the method includes sequencing at least a portion of the first oligonucleotide strand while the first oligonucleotide strand is a part of a double-stranded bridged structure. FIG. 7B shows the workflow of such a method. The workflow includes providing a first presequencing complex PSC1. The first pre-sequencing complex PSC1 includes a first oligonucleotide strand 30 having a free 3′ end and a 5′ end bound to the surface 15. The first oligonucleotide strand includes a 3′ portion 20′ and an extension primer binding site 32. The first pre-sequencing complex also includes surface oligonucleotide 20 having a free 3′ end and a 5′end bound to the surface 15. The first 3′ portion 20′ of the first oligonucleotide strand 30 is hybridized to at least a portion of a surface oligonucleotide 20 forming a bridged structure. The first pre-sequencing complex PSC1 also includes an extension primer 70 bound to the extension primer binding site 32. The extension primer 70 has a free 3′ end and a free 5′ end and include at least one cleavage site 72. The first presequencing complex PCS1 may be provided upon the completion of bridge amplification (described herein).

In step A of FIG. 7B, the extension primer 70 is enzymatically extended from the free 3′ end using the first oligonucleotide strand 30 as a template to create as second oligonucleotide strand 30′ and a second pre-sequencing complex PSC2. The second pre-sequencing complex PSC2 includes the first oligonucleotide strand 30 hybridized to the second oligonucleotide strand 30′ in a double-stranded bridge structure. The second oligonucleotide strand 30′ has a free 3′ end, a free 5′ end, and includes the extension primer 70.

In step B of FIG. 7B, the extension primer 70 of the second oligonucleotides strand 30′ is cleaved at the cleavage site 72 creating a sequencing complex SC. Any cleavage method may be used such as those discussed herein. The sequence complex SC includes a cleaved first portion 30′c(i) and a cleaved second portion 30′c(ii) of the second oligonucleotide strand 30′. The cleaved first portion 30′c(i) includes a free 5′ end and a free 3′ end having a 3′ hydroxyl. The cleaved second portion 30′c(ii) includes a free 5′ end and a free 3′ end. The sequencing complex also includes the cleaved first portion 30′c(i) and cleaved second portion 30′c(ii) of the second oligonucleotide strand hybridized to the first oligonucleotide 30 in a double-stranded bridged structure.

In step C of FIG. 7B, at least a portion of the first oligonucleotide strand 30 is sequenced as a first read region R1. Sequencing may include sequencing by synthesis where the free 3′ end of the cleaved first portion 30′c(i) of the second oligonucleotide strand 30′ serves as a sequencing primer. As such, the cleaved first portion 30′c(i) of the second oligonucleotide strand 30′ is enzymatically extended in the 5′ to 3′ direction thereby creating a portion of a third oligonucleotide strand 30′a that is complementary to and hybridized to the first oligonucleotide strand 30. The portion of the third oligonucleotide strand 30′a generated during sequencing is the first read region R1. The enzymatic extension uses the first oligonucleotide strand 30 as the template and the cleaved first portion 30′c(i) of the second oligonucleotide strand 30′ as the sequencing primer. The double-stranded sequencing may occur via strand displacement or nick translation.

In some cases where the surface 15 includes an array, individual complexes may undergo the double-stranded sequencing method 200 according to FIG. 7A, FIG. 7B, or both. For example, the first oligonucleotide strand 30 of FIG. 7A may include 3′ portion capable of hybridizing to a surface oligonucleotide to form a bridge structure. Additionally, each step of the double-stranded sequencing method 200 may occur in a bridged structure or a linear structure; that is, the double-stranded complex (or single stranded complex prior to extension primer extension) may transition from a bridged structure to a linear structure one or more times during the completion of method 200.

FIG. 3 is a flow chart illustrating an overview of a third double-stranded sequencing method (300) consistent with some embodiments of the present disclosure. The method includes providing a first oligonucleotide strand having a 5′ end bound to a surface (302). The method further includes hybridizing an extension primer to a portion of the first oligonucleotide strand (304). The extension primer includes a free 3′ end, a free 5′ end, and two or more cleavage sites. The method further includes extending the extension primer from the free 3′ end using the first oligonucleotide strand as a template to produce a second oligonucleotide strand (306). The second oligonucleotide strand includes the extension primer and an extended portion. The second oligonucleotide strand is hybridized to the first oligonucleotide strand. The method further includes cleaving the two or more cleavage sites in the extension primer to produce multiple fragments and a second portion of the second oligonucleotide strand (308). The second portion of the second oligonucleotide strand is hybridized to the first oligonucleotide strand and includes the extended second portion. The method further includes hybridizing a sequencing primer to the first oligonucleotide strand (310). The sequencing primer includes a free 5′ end and a free 3′ end. Additionally, the sequencing primer hybridizes to a region of the first oligonucleotide strand to which at least a portion of the extension primer hybridized. The method further includes extending the sequencing primer from the free 3′ end using the first oligonucleotide as a template and sequencing at least a portion of the first oligonucleotide strand as the sequencing primer is extended (312).

In some embodiments of the third double-stranded sequencing method 300, step 302 further comprises providing a surface oligonucleotide having a free 3′ end and having a 5′ end bound to the surface. In such embodiments, the first oligonucleotide strand includes a 3′ portion that is at least partially complementary to at least a portion of the surface oligonucleotide. As such, the 3′ portion of the first oligonucleotide strand is hybridized to the surface oligonucleotide forming a single stranded bridge structure. In step 306, the single stranded bridged structure is converted into a double-stranded bridged structure.

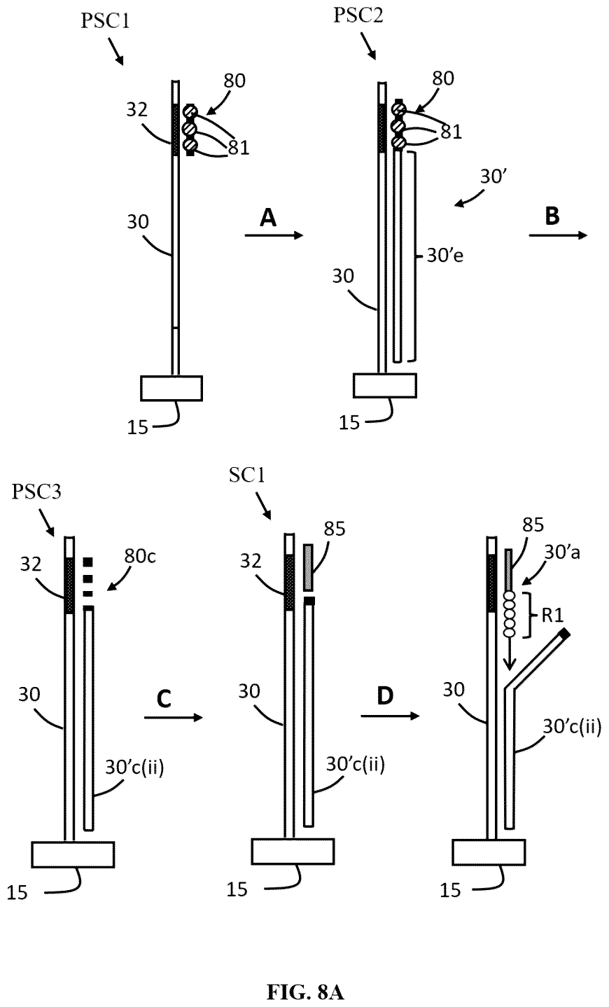

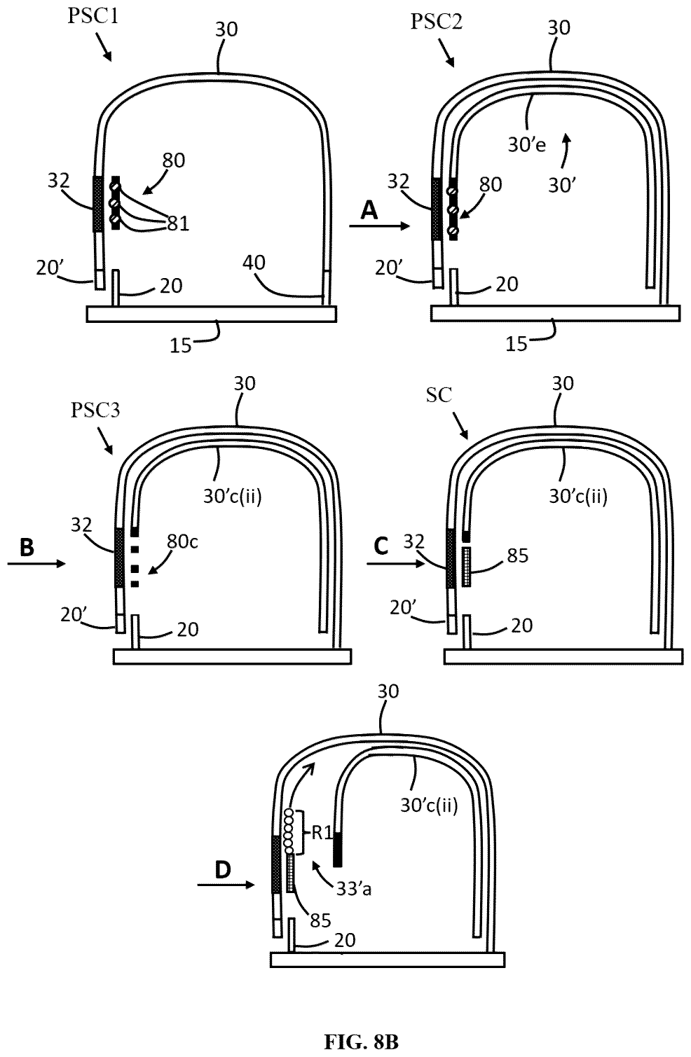

FIGS. 8A-8B provide a schematic overview of a double-stranded surface sequencing method consistent with some embodiments of the present disclosure, for example, the third double-stranded sequencing method of FIG. 4. The workflow of FIG. 8A includes providing a pre-sequencing complex PSC1 (step 302 of method 300). Pre-sequencing complex PSC1 includes a first oligonucleotide strand 30 having a free 3′ end and a 5′ end bound to a surface 15. The first oligonucleotide strand 30 includes an extension primer binding region 32. The extension primer binding region 32 may be located at any location on the first oligonucleotide strand 30. In some embodiments where the first oligonucleotide strand 30 includes adapters, the adapter sequence proximate the 3′ end of the first oligonucleotide strand 30 includes the extension primer binding region 32. The first pre-sequencing complex PSC1 also includes an extension primer 80 hybridized to at least a portion of the extension primer binding region 32 of the first oligonucleotide strand 30. The extension primer 85 has a free 3′ end and a free 5′ end and includes at least two cleavage sites 81.

In step A of FIG. 8A, the extension primer 80 is enzymatically extended from the free 3′ end using the first oligonucleotide strand 30 as a template to create a second oligonucleotide strand 30′ and a second pre-sequencing complex PSC2. The second pre-sequencing complex PSC2 includes the first oligonucleotide strand 30 hybridized to the second oligonucleotide strand 30′. The second oligonucleotide strand 30′ has a free 3′ end, a free 5′ end, and includes the extension primer 80 and an extended portion 30′e.

In step B of FIG. 8A, the extension primer 80 of the second oligonucleotides strand 30′ is cleaved at two or more of the cleavage sites 81 creating a third pre-sequencing complex PSC3. Any cleavage method or combination of cleavage methods may be used such as those described herein. The third pre-sequencing complex PSC3 includes multiple fragments 80c (multiple extension primer fragments) and a cleaved second portion 30′c(ii) of the second oligonucleotide strand 30′. The second portion 30′c(ii) includes the extended portion 30′e. The second portion 30′c(ii) includes a free 5′ end and a free 3′ end. The second portion 30′c(ii) is hybridized to the first oligonucleotide strand 30. One or more of the multiple fragments 80c may be hybridized to the first oligonucleotide strand 30.

In step C of FIG. 8A, a sequencing primer is hybridized to the first oligonucleotide strand 30 to form a sequencing complex SC. The sequencing complex SC includes the first oligonucleotide strand 30′ hybridized to the sequencing primer 85 and the second portion 30′c(ii) of the second oligonucleotide strand 30′. The sequencing primer 85 is hybridized to a region of the first oligonucleotide strand 30 to which at least a portion of the extension primer 80 hybridized to; that is, the sequencing primer is hybridized to at least a portion of the extension primer binding region 32 of the first oligonucleotide strand 30′.

In some embodiments, step C of FIG. 8A also includes dehybridizing the multiple fragments 80c from the first oligonucleotide strand 30. Dehybridization may be accomplished by exposing the third pre-sequencing complex PSC3 to an elevated temperature. The elevated temperature can be chosen such that the multiple fragments 80c dehybridize from the first oligonucleotide strand while the second portion 30′c(ii) remains hybridized to the first oligonucleotide strand 30. Alternatively or additionally, the sequencing primer 85 can be added to compete off the multiple fragments 80c.

In step D of FIG. 8A, at least a portion of the first oligonucleotide strand 30 is sequenced as a first read region R1. Sequencing may include sequencing by synthesis where the free 3′ end of the sequencing primer 85 serves as a sequencing primer. As such, the sequencing primer 85 is enzymatically extended in the 5′ to 3′ direction thereby creating a portion of a third oligonucleotide strand 30′a that is complementary to and hybridized to the first oligonucleotide strand 30. The portion of the third oligonucleotide strand 30′a generated during sequencing is the first read region R1. The enzymatic extension uses the first oligonucleotide strand 30 as the template.

Some embodiments of the third double-stranded sequencing method (300) include sequencing at least a portion of the first oligonucleotide strand while the first oligonucleotide strand is a part of a double-stranded bridged structure. FIG. 8B shows the workflow of such a method. The workflow includes providing a first presequencing complex PSC1. The first pre-sequencing complex PSC1 includes a first oligonucleotide strand 30 having a free 3′ end and a 5′ end bound to the surface 15. The first oligonucleotide strand includes a 3′ portion 20′ and an extension primer binding site 32. The first pre-sequencing complex also includes surface oligonucleotide 20 having a free 3′ end and a 5′ end bound to the surface 15. The first 3′ portion 20′ of the first oligonucleotide strand 30 is hybridized to at least a portion of the surface oligonucleotide 20 forming a bridged structure. The first pre-sequencing complex PSC1 also includes an extension primer 80 bound to the extension primer binding site 32. The extension primer 80 has a free 3′ end and a free 5′ end and includes two or more cleavage sites 81.

In step A of FIG. 8B, the extension primer 80 is enzymatically extended from the free 3′ end using the first oligonucleotide strand 30 as a template to create a second oligonucleotide strand 30′ and a second pre-sequencing complex PSC2. The second pre-sequencing complex PSC2 includes the first oligonucleotide strand 30 hybridized to the second oligonucleotide strand 30′ in a double-stranded bridged structure. The second oligonucleotide strand 30′ has a free 3′ end, a free 5′ end, and includes the extension primer 80 and an extended portion 30′e.

In step B of FIG. 8B, the extension primer 80 of the second oligonucleotides strand 30′ is cleaved at two or more of the cleavage sites 81 creating a third pre-sequencing complex PSC3. Any cleavage method or combination of cleavage methods may be used such as those described herein. The third pre-sequencing complex PSC3 includes multiple fragments 80c (multiple extension primer fragments) and a cleaved second portion 30′c(ii) of the second oligonucleotide strand 30′. The second portion 30′c(ii) includes the extended second portion 30′e. The second portion 30′c(ii) includes a free a free 5′ end and a free 3′ end. The second portion 30′c(ii) is hybridized to the first oligonucleotide strand. One or more of the multiple fragments 80c may be hybridized to the first oligonucleotide strand 30.

In step C of FIG. 8B, a sequencing primer 85 is hybridized to the first oligonucleotide strand 30 to form a sequencing complex SC. The sequencing complex SC includes the first oligonucleotide strand 30′ hybridized to the sequencing primer 85 and the second portion 30′c(ii) of the second oligonucleotide strand 30′. The sequencing primer 85 is hybridized to a region of the first oligonucleotide strand 30 to which at least a portion of the extension primer 80 hybridized to; that is, the sequencing primer is hybridized to at least a portion of the extension primer binding region 32 of the first oligonucleotide strand 30′. In some embodiments, step C of FIG. 8B also includes dehybridizing the multiple fragments 80c from the first oligonucleotide strand 30. Dehybridization may be accomplished by any means such as those disclosed herein.

In step D of FIG. 8B, at least a portion of the first oligonucleotide strand 30 is sequenced as a first read region R1. Sequencing may include sequencing by synthesis where the free 3′ end of the sequencing primer 85 serves as a sequencing primer. As such, the sequencing primer 85 is enzymatically extended in the 5′ to 3′ direction thereby creating a portion of a third oligonucleotide strand 30′a that is complementary to and hybridized to the first oligonucleotide strand 30. The portion of the third oligonucleotide strand 30′a generated during sequencing is the first read region R1. The enzymatic extension uses the first oligonucleotide strand 30 as the template. Double-stranded sequencing may occur via strand displacement or nick translation.

Similar to method 200, In some cases where the surface 15 includes an array, individual complexes may undergo the double-stranded sequencing method 300 according to FIG. 8A, FIG. 8B, or both. For example, the first oligonucleotide strand 30 of FIG. 7B may include 3′ portion capable of hybridizing to a surface oligonucleotide to form a bridge structure. Additionally, each step of the double-stranded sequencing method 200 may occur in a bridged structure or a linear structure; that is, the double-stranded complex (or single stranded complex prior to extension primer extension) may transition from a bridged structure to a linear structure one or more times during the completion of method 300.

The sequencing methods disclosed herein may be performed sequentially. For example, method 200 or 300 may be used to sequence at least a portion of the first oligonucleotide strand and method 100 may be used to sequence at least a portion of the third oligonucleotide strand generated during method 200 or 300. In such embodiments, the first oligonucleotide strand or the third oligonucleotide strand of method 200 or 300 includes a nuclease binding region allowing the nuclease to cleave the first oligonucleotide strand creating a cleaved oligonucleotide strand that can be used as a sequencing primer.

In some embodiments, a kit comprises all reagents needed for sequencing polynucleotides according to one or more of the double-stranded sequencing methods described herein. Any of the reagents disclosed herein may be included in the kit. For example, the kit may include a polymerase and labeled, blocked nucleotides. The kit may include unblocked nucleotides for extension, for example, after the first sequencing read. The kit may include a cleavage reagent and, if needed, a conversion reagent as described herein. The kit may include any or all reagents needed to accomplish chemical cleavage such as for example, OsO₄ or precursor compounds used to generate OsO₄ in situ, K₃[Fe(CN)₆], peroxide, N-methylmorpholine N-oxide (NMO), 12, silver salts, or any combination thereof. The kit may include reagents to carry out the pre-sequencing methods described herein. For example, the kit may comprise enzymes and nucleotides for amplification and cluster formation. The kit may comprise a flap nuclease or a polymerase-flap nuclease construct for use in embodiments of double-stranded surface sequencing methods. The kit may include extension primers and/or sequencing primers.

Throughout this application, various publications are referenced. The disclosures of these publications in their entireties are hereby incorporated by reference into this application.

### EXAMPLES OF EMBODIMENTS

The invention is defined in the claims. However, below there is provided a non-exhaustive listing of non-limiting examples of embodiments. Any one or more of the features of these aspects may be combined with any one or more features of another example, embodiment, or aspect described herein.

Embodiment 1 is a nucleotide sequencing method comprising: providing a first surface-bound double-stranded oligonucleotide complex comprising a first oligonucleotide strand and a second oligonucleotide strand, wherein the first oligonucleotide strand has a 5′ end bound to a surface; exposing the first surface-bound double-stranded oligonucleotide complex to a nuclease to cleave the second oligonucleotide strand and produce a cleaved first portion and a cleaved second portion of the second oligonucleotide strand, wherein the cleaved first and second portions are hybridized to the first oligonucleotide strand, and wherein the first cleaved portion comprises a free 3′ end; and extending the first cleaved portion from the free 3′ end using the first oligonucleotide strand as a template and sequencing at least a portion of the first oligonucleotide strand as the cleaved first portion is extended.

Embodiment 2 is the nucleotide sequencing method of Embodiment 1, wherein the cleaved first portion of the second oligonucleotide strand has a 5′ end bound to the surface.

Embodiment 3 is the nucleotide sequencing method of Embodiment 1 or 2, wherein extending the surface bound cleaved first portion generates a second surface-bound double-stranded oligonucleotide complex comprising the first oligonucleotide strand and a nascent third oligonucleotide strand comprising the surface bound cleaved first portion.

Embodiment 4 is the nucleotide sequencing method of any one of Embodiments 1 to 3, wherein a ribonucleoprotein comprises a guide RNA (gRNA) and the nuclease, and wherein the gRNA hybridizes to a portion of the first oligonucleotide strand or a portion of the second oligonucleotide strand.

Embodiment 5 is the is the nucleotide sequencing method of Embodiment 4, wherein the nuclease is a Cas nuclease or a Cas nuclease variant.

Embodiment 6 is the is the nucleotide sequencing method of Embodiment 4 or 5, wherein the nuclease is Cas9, spCas9, spCas9HF, or a variant thereof.

Embodiment 7 is the nucleotide sequencing method of any one of Embodiments 1 to 6, wherein extending the cleaved first portion from the free 3′ end results in the displacement of at least a 5′ portion of the cleaved second portion from the first oligonucleotide strand.

Embodiment 8 is the nucleotide sequencing method of any one of Embodiments 1 to 7, wherein extending the cleaved first portion from the free 3′ end further comprises: removing nucleotides and/or oligonucleotides from the cleaved second portion using a flap nuclease.

Embodiment 9 is the nucleotide sequencing method of Embodiment 8, wherein the flap nuclease is operably linked to a second protein.

Embodiment 10 is the nucleotide sequencing method of Embodiment 9, wherein the second protein comprises a polymerase.

Embodiment 11 is the nucleotide sequencing method of Embodiment 10, wherein the polymerase comprises GINS-associated nuclease (GAN), Taq DNA polymerase, Bst DNA polymerase, or FEN1.

Embodiment 12 is the nucleotide sequencing method of Embodiment 9, wherein the second protein comprises a DNA binding domain.

Embodiment 13 is the nucleotide sequencing method of Embodiment 12, wherein the DNA binding protein comprises a dsDNA binding domain.

Embodiment 14 is the nucleotide sequencing method of Embodiment 13, wherein the DNA binding domain comprises a sequence-nonspecific DNA binding domain.

Embodiment 15 is the nucleotide sequencing method of Embodiment 14, wherein the DNA binding protein comprises Sso7d or portion thereof having affinity for DNA.

Embodiment 16 is the nucleotide sequencing method of any one of Embodiments 9 to 15, wherein the flap nuclease and the second protein are coupled by a linker of SEQ ID NO. 5, SEQ ID NO. 6, SEQ ID NO. 7, or SEQ ID NO. 10.

Embodiment 17 is nucleotide sequencing of any one of Embodiments 2 through 16, further comprising: exposing the second surface-bound double-stranded oligonucleotide complex to a second nuclease to cleave the first oligonucleotide strand and produce cleaved third and cleaved fourth portions of the first oligonucleotide strand, wherein the cleaved third and cleaved fourth portions are hybridized to the third oligonucleotide strand, and wherein the cleaved third portion comprises a surface bound 5′ end and a free 3′ end; and extending the cleaved third portion from the free 3′ end using the third oligonucleotide strand as a template and sequencing at least a portion of the third oligonucleotide strand as the cleaved third portion is extended.

Embodiment 18 is a nucleotide sequencing method comprising: providing a first oligonucleotide strand having a 5′ end bound to a surface; hybridizing an extension primer to a portion of the first oligonucleotide strand, the primer having a free 3′ end, a free 5′ end, and a cleavage site; extending the extension primer from the free 3′ end using the first oligonucleotide strand as a template to produce a second oligonucleotide strand hybridized to the surface-bound first oligonucleotide strand and comprising the extension primer; cleaving the extension primer of the second oligonucleotide strand at the cleavage site to produce cleaved first and second portions of the second oligonucleotide strand, wherein the cleaved first and second portions are hybridized to the first oligonucleotide strand, and wherein the cleaved first portion comprises a free 3′ end; and extending the cleaved first portion from the free 3′ end using the first oligonucleotide strand as a template and sequencing at least a portion of the first oligonucleotide strand as the cleaved first portion is extended.

Embodiment 19 is the nucleotide sequencing method of Embodiment 18, wherein the first oligonucleotide strand comprises a 3′ portion, and wherein the method further comprises providing a surface oligonucleotide having a free 3′ end and having a 5′ end bound to the surface, wherein the 3′ portion of the first oligonucleotide is hybridized to at least a portion of the surface oligonucleotide.

Embodiment 20 is the nucleotide sequencing method of Embodiment 18 or 19, wherein cleaving the extension primer of the second oligonucleotide strand at the cleavage site further comprises: removing a first excisable base from the primer to produce the cleaved first portion; or treating the extension primer of the second oligonucleotide strand with one or more dihydroxylation reagents to produce the cleaved first portion.

Embodiment 21 is the nucleotide sequencing method of any one of Embodiments 18 to 20, wherein extending the cleaved first portion from the free 3′ end results in the displacement of at least a 5′ portion of the cleaved second portion from the first oligonucleotide strand.

Embodiment 22 is the nucleotide sequencing method of any one of Embodiments 18 to 21, wherein extending the cleaved first portion from the free 3′ end further comprises: removing nucleotides and/or oligonucleotides from the cleaved second portion using a flap nuclease.

Embodiment 23 is the nucleotide sequencing method of Embodiment 22, wherein the flap nuclease is operably linked to a second protein.

Embodiment 24 is the nucleotide sequencing method of Embodiment 23, wherein the second protein comprises a polymerase.

Embodiment 25 is the nucleotide sequencing method of Embodiment 24, wherein the polymerase comprises GINS-associated nuclease (GAN), Taq DNA polymerase, Bst DNA polymerase, or FEN1.

Embodiment 26 is the nucleotide sequencing method of Embodiment 23, wherein the second protein comprises a DNA binding domain.

Embodiment 27 is the nucleotide sequencing method of Embodiment 26, wherein the DNA binding protein comprises a dsDNA binding domain.

Embodiment 28 is the nucleotide sequencing method of Embodiment 26 or 27, wherein the DNA binding domain comprises a sequence-nonspecific DNA binding domain.

Embodiment 29 is the nucleotide sequencing method of any one of Embodiments 26 to 29, wherein the DNA binding domain comprises Sso7d or a portion thereof having DNA affinity.

Embodiment 30 is the nucleotide sequencing method of any one of Embodiments 23 to 29, wherein the flap nuclease and the second protein are coupled by a linker of SEQ ID NO. 5, SEQ ID NO. 6, SEQ ID NO. 7, or SEQ ID NO. 10.

Embodiment 31 is a nucleotide sequencing method comprising: providing a first oligonucleotide strand having a 5′ end bound to a surface; hybridizing an extension primer to a portion of the first oligonucleotide strand, the extension primer comprising two or more cleavage sites and comprising a free 3′ end and a free 5′ end; extending the extension primer from the free 3′ prime end using the first oligonucleotide strand as a template to produce a second oligonucleotide strand hybridized to the surface-bound first strand, the second oligonucleotide strand comprising the extension primer and an extended second portion; cleaving the extension primer of the second oligonucleotide at the two or more cleavage sites to produce multiple fragments and a second portion of the second oligonucleotide strand, wherein the second portion of the second oligonucleotide strand comprises the extended second portion, wherein the second portion is hybridized to the first oligonucleotide strand; hybridizing a sequencing primer to the first oligonucleotide strand, wherein at least a portion of the sequencing primer hybridizes to a region of the first oligonucleotide strand to which at least a portion of the first primer hybridized, the sequencing primer comprising a free 3′ end and a free 5′ end; extending the sequencing primer from the free 3′ end using the first oligonucleotide strand as a template and sequencing at least a portion of the first oligonucleotide strand as the sequencing primer is extended.

Embodiment 32 is the nucleotide sequencing method of Embodiment 31, wherein the first oligonucleotide strand comprises a 3′ portion, and wherein the method further comprising providing a surface oligonucleotide strand having a free 3′ end and a 5′ end bound to the surface, wherein the 3′ portion of the first oligonucleotide strand is hybridized to at least a portion of the surface oligonucleotide.

Embodiment 33 is the nucleotide sequencing method of Embodiment 31 or 22, wherein cleaving the extension primer of the second oligonucleotide strand at the two or more cleavage sites to produce multiple fragments further comprises dehybridizing at least one of the multiple primers fragments.

Embodiment 34 is the nucleotide sequencing method of Embodiment 33, wherein dehybridizing at least one of the multiple fragments comprises exposing the second oligonucleotide strand to an elevated temperature.

Embodiment 35 is the nucleotide sequencing method of any one of Embodiments 31 to 34, wherein extending the sequencing primer from the free 3′ end results in the displacement of at least a 5′ portion of a second portion of the second oligonucleotide strand from the first oligonucleotide strand.

Embodiment 36 is the nucleotide sequencing method of Embodiment 35, wherein the flap nuclease is operably linked to a second protein.

Embodiment 37 is the nucleotide sequencing method of Embodiment 36, wherein the second protein comprises a polymerase.

Embodiment 38 is the nucleotide sequencing method of Embodiment 37, wherein the polymerase comprises GINS-associated nuclease (GAN), Taq DNA polymerase, Bst DNA polymerase, or FEN1.

Embodiment 39 is the nucleotide sequencing method of Embodiment 36, wherein the second protein comprises a DNA binding domain.

Embodiment 40 is the nucleotide sequencing method of Embodiment 39, wherein the DNA binding protein comprises a dsDNA binding domain.

Embodiment 41 is the nucleotide sequencing method of Embodiment 40, wherein the DNA binding domain comprises a sequence-nonspecific DNA binding domain.

Embodiment 42 is the nucleotide sequencing method of any one of Embodiments 39 to 41, wherein the DNA binding domain comprises Sso7d or a portion thereof having DNA affinity.

Embodiment 43 is the nucleotide sequencing method of any one of Embodiments 36 to 42, wherein the flap nuclease and the second protein are coupled by a linker of SEQ ID NO. 5, SEQ ID NO. 6, SEQ ID NO. 7, or SEQ ID NO. 10.

Embodiment 44 is a nucleotide sequencing method comprising: (a) providing a surface-bound double-stranded oligonucleotide complex comprising a first oligonucleotide strand, a second oligonucleotide strand hybridized to the first nucleotide strand, and a primer hybridized to the first oligonucleotide strand, wherein the first oligonucleotide strand has a 5′ end bound to a surface, and wherein a free 3′ end of the primer is hybridized to a nucleotide of the first oligonucleotide strand that is 3′ of a nucleotide of the first oligonucleotide strand to which a 5′ end of the second oligonucleotide strand is hybridized; (b) extending the primer from the free 3′ end using the first oligonucleotide strand as a template and sequencing at least a portion of the first oligonucleotide strand via sequencing by synthesis as the primer is extended; and (c) nicking the second strand to remove a 5′ portion of the second strand before, during, or after one or more nucleotides are added to extend the primer in step (b).

Embodiment 45 is the nucleotide sequencing method of Embodiment 44, wherein the second strand is nicked in step (c) before, during, or after each nucleotide is added to extend the primer in step (b).

Embodiment 45 is the nucleotide sequencing method of Embodiment 44, wherein the second strand is nicked in step (c) after more than one nucleotide is added to extend the primer in step (b).

### EXAMPLES

The polymerases used in the examples can be found in US Patent Application Number U.S. Ser. No. 16/703,569 (U.S. Ser. No. 11/001,816B2) (Pol(X)).

### Example 1

Illustration of two double-stranded sequencing techniques commensurate with one or more embodiments of sequencing methods 100 or 200 of the present disclosure.

Sequencing performance of sequencing techniques commensurate with embodiments of method 100 or method 200 were evaluated using an Illumina iSeq100 sequencing by synthesis system. All samples prepared and sequenced used a DNA library containing a portion of the human genome.

Three samples were prepared and sequenced. A control sample (Run A), was prepared by forming DNA clusters using the standard iSeq100 protocol according to manufacturer instructions. Single stranded sequencing by synthesis was carried out as described in U.S. Pat. No. 11,293,061B2 and US Patent Pub. No. US 2021/0403500A1.

A second sample (Run B) was prepared and sequenced using a technique commensurate with embodiments of method 100 of the present disclosure. In this sample, DNA clusters were amplified using the standard iSeq100 protocol, but were not linearized. This produced clusters of oligonucleotide complexes as shown in the first step of FIG. 6B (e.g., PSC1).

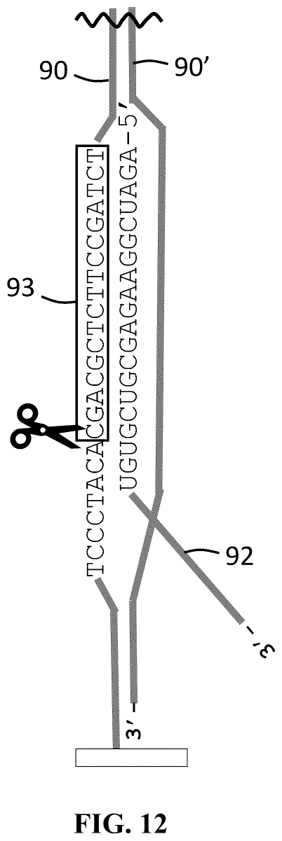

Method 100 was then used as shown in FIG. 6B. A complex of Cas9 D10A nickase from Integrated DNA Technologies with gRNA was incubated with the clusters at 500 nM for 25 min at 40° C. to cleave one of the strands (e.g., step B of FIG. 6B). gRNA sequence was mA*mG*mA* rUrCrG rGrArA rGrArG rCrGrU rCrGrU rGrUrG rUrUrU rUrArG rArGrC rUrArG rArArA rUrArG rCrArA rGrUrU rArArA rArUrArArGrG rCrUrA rGrUrC rCrGrU rUrArU rCrArA rCrUrU rGrArA rArArA rGrUrG rGrCrA rCrCrG rArGrU rCrGrG rUrGrC mU*mU*mU* rU, with a crRNA sequence complementary to the target DNA of AGAUCGGAAGAGCGUCGUGU. This is illustrated in FIG. 12. gRNA 92 hybridized to target region 93 of the first oligonucleotide strand 90 that is hybridized to a second oligonucleotide strand 90′. The predicted cut site is marked.

Sequencing by synthesis was carried out as described in U.S. Pat. No. 11,293,061B2 and US Patent Pub. No. US 2021/0403500A1. Nick translation was performed using Pol(X) coupled to GINS-associated nuclease (GAN) to incorporate and nick simultaneously. The GAN nuclease was from T. nautili and the polymerase and GAN nuclease were connected by a AALGGAAAAAAS linker sequence. 17 cycles of SBS were performed without imaging at the beginning of the run to get through the Cas9 target region 93.

A third sample (Run C) was prepared and sequenced using a technique commensurate with embodiments of method 200 of the present disclosure. In this sample, DNA clusters were produced using the standard iSeq100 protocol, replacing the standard Read 1 primer with a primer containing an uracil. The primer was elongated to form a bridged double stranded oligonucleotide complex to produce clusters of double stranded oligonucleotide complexes as shown in the first step of FIG. 7B (PSC2). The uracil specific excision reagent (USER; available from New England BioLabs, Ipswich, MA) was incubated with the clusters to cleave the uracil from the primer (e.g., Step B of FIG. 7B) and to form a sequencing primer having a free 5′ end that includes at least a portion of the initial primer. Sequencing by synthesis was performed as for Run B (i.e., using Pol(X) coupled to GAN to incorporate nucleotides and nick simultaneously).

Table 5 shows sequencing metrics for Runs A, B and C. Starting intensity is the 90^{th} percentile intensity of the first Green channel image of SBS Cycle 6 for each run. Runs B and C had starting intensity comparable to baseline, indicating that techniques commensurate with embodiments of methods 100 and 200 were able to prepare DNA for sequencing successfully. Additionally, the percent passing filter (% PF) and percent of base calls having a Q score of 30 or greater (%>Q30) were evaluated. Q scores are used to estimate the accuracy of base calling during sequencing. Q is defined as −10×log 10(e) where e is the estimated probability of the base call being wrong. Higher Q scores indicate a small probability of error. Generally, Q scores above 30 indicate high accuracy. The % Q30 and % PF for Runs B and were comparable to baseline, indicating that sequencing using the methods of Run B and Run C was successful.

TABLE 5

| Sequencing | | | | | % soft clipping |
|---|---|---|---|---|---|
| Run | | Starting | | % > | in G-quadruplex |
| Reference | Run type | intensity | % PF | Q30 | regions |
| Run A | Baseline | 255 | 69.9 | 85.1 | 37.9 |
| Run B | Method 100 | 275 | 62.3 | 83.0 | 17.3 |
| Run C | Method 200 | 290 | 53.4 | 83.1 | 18.0 |

The DRAGEN Secondary Analysis aligner (available from Illumina) was used to align the sequenced DNA to the reference. Bases at the beginning or end of a Read that do not align to the reference are recorded by the aligner as ‘soft clipped’. The greater the percentage of soft clipping, the greater the number of sequencing errors. The ability to sequence regions containing a G quadruplex was assessed using the % of DNA bases in a predicted G quadruplex region that are soft clipped. Only predicted G quadruplex regions that had greater than 10% soft clipping in Run A, but sequenced successfully in the opposite strand to the G quadruplex were included. The percentage soft clipping in these regions for Runs A, B and C were 37.9, 17.3 and 18.0 respectively. This demonstrates improved sequencing performance in G quadruplex regions in Run B and Run C compared with standard single stranded sequencing by synthesis.

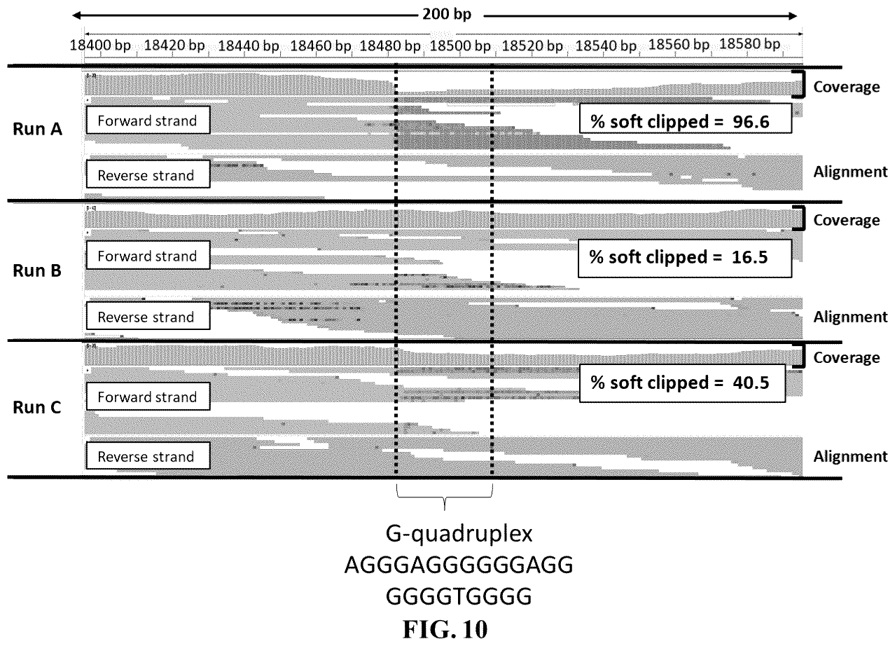

An example of improved sequencing performance at a G quadruplex region with Runs B and C is shown in the Broad Institute Integrative Genomics Viewer plot in FIG. 10. Run A had a high number of errors (darker colour) in the Forward strand, which contained a G quadruplex, with 96.6% soft clipping for the region shown. The reverse strand, which does not contain a G quadruplex, had very few errors. Runs B and C, which used methods 100 quad regions and 200 respectively, had 16.5% and 40.5% soft clipping in the Forward strand respectively in this region.

### Example 2

Illustration of a double-stranded sequencing techniques commensurate with one or more embodiments of sequencing method 300 of the present disclosure.

Sequencing performance of a sequencing technique commensurate with embodiments of method 300 was compared to a standard single stranded sequencing using an Illumina NextSeq2000 sequencing by synthesis system. All samples produced and sequenced used a DNA library containing a portion of the human genome.

Three samples were prepared and sequenced. A control sample (Run D) was prepared by forming DNA clusters using the standard NextSeq2000 protocol according to manufacturer instructions. Single stranded sequencing by synthesis was carried out as described in U.S. Pat. No. 11,293,061B2 and US Patent Pub. No. US 2021/0403500A1.

A second sample (Run E) was prepared and sequenced using a technique commensurate with embodiments of method 300 of the present disclosure. In this sample, DNA clusters were produced using the standard NextSeq2000 protocol, replacing the Read 1 primer with a primer containing four uracils. The primer was elongated to form a bridged double stranded oligonucleotide complex to produce clusters of double stranded oligonucleotide complexes as shown in the first step of FIG. 8B (PSC2). The uracil specific excision reagent (USER; available from New England BioLabs, Ipswich, MA) was incubated with the clusters to cleave the uracils from the primer (e.g., Step B of FIG. 8B) to produce multiple fragments (e.g., step B in FIG. 8B). Sequencing by synthesis was carried out as described in U.S. Pat. No. 11,293,061B2 and US Patent Pub. No. US 2021/0403500A1. Nick translation was performed using Pol(X) coupled to GAN to incorporate and nick simultaneously.

Table 6 shows sequencing metrics for Runs D, and E. Starting intensity % PF and %>Q30 for Run E were only slightly lower than baseline, indicating that sequencing using the techniques of Run E was successful. Percentage soft clipping in the G quadruplex regions for Runs D and E was 48.3 and 25.9% respectively. This demonstrates that the double stranded sequencing technique of Run E gives improved sequencing performance of G quadruplex regions, despite slightly lower primary sequencing metrics.

TABLE 6

| | | | | | % soft |
|---|---|---|---|---|---|
| Sequencing | | | | | clipping in |
| Run | | Starting | % | | G-quadruplex |
| Reference | Run type | intensity | PF | % > Q30 | regions |
| Run D | Baseline | 342 | 83.7 | 92.5 | 48.3 |
| Run E | Method 300 | 325 | 69.1 | 75.6 | 25.9 |

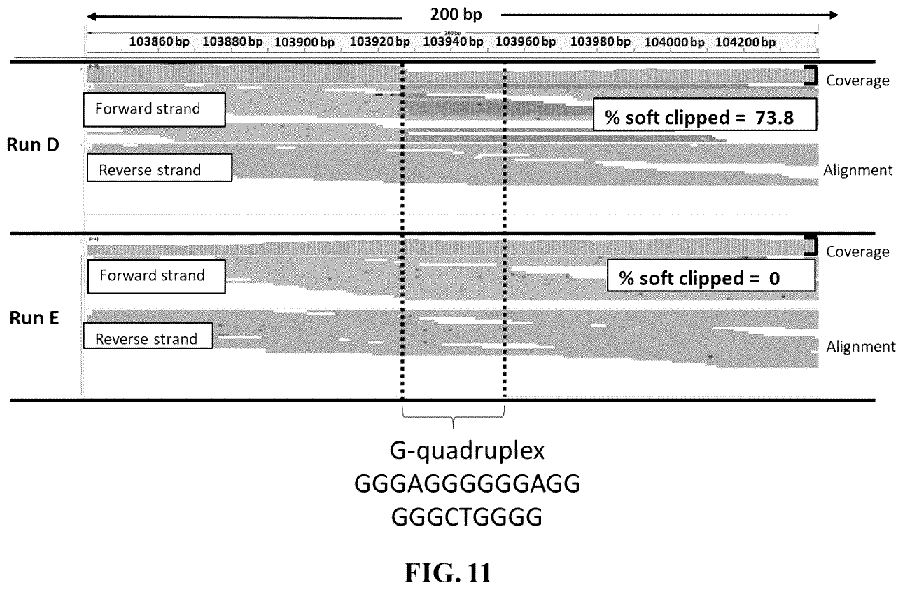

An example of improved sequencing performance at a G quadruplex region with Run E as compared to Run D is shown in the Broad Institute Integrative Genomics Viewer plot in FIG. 11. Run D had a high number of errors (darker color) in the Forward strand, which contained a G quadruplex, with 73.8% soft clipping. The reverse strand, which does not contain a G quadruplex, had very few errors. Run E, had 0% soft clipping in the Forward strand.

### Example 3

Illustration of a double-stranded sequencing technique commensurate with one or more embodiments of sequencing method 100 of the present disclosure. Sequencing performance of sequencing techniques commensurate with embodiments of method 100 were compared. All samples produced and sequenced used a DNA monotemplate with the sequence 5′-AATGATACGGCGACCACCGAGATCTACACCTAGGAGCTAAAGCGAGATCGG AAGAGCGTCGTGTAGGGAAAGAGTGTATCTCGTATGCCGTCTTCTGCTTG-3′ (SEQ ID NO: 8). This had sequences complementary to the Illumina flowcell surface primers, and a generic DNA sequence in the middle.

A control sample (Run F) was prepared and sequenced using a technique commensurate with embodiments of method 100 of the present disclosure. In this sample, DNA clusters were produced using the standard NextSeq2000 protocol, up to the linearization step. Linearization was carried out using the standard NextSeq2000 protocol to produce clusters of double stranded oligonucleotide complexes as shown in the second step of FIG. 6B (PSC1). The 3′ phosphor group on surface DNA primer 30′c(i) was converted to 3′-OH using standard NextSeq2000 deprotection protocol. Ssequencing by synthesis was carried out as described in U.S. Pat. No. 11,293,061B2 and US Patent Pub. No. US 2021/0403500A1. 35 cycles of SBS were performed using 3 μM Pol(X) without the addition or presence of a flap nuclease. A 1 M NaCl salt wash was performed after each incorporation step. The DNA strand containing the created flap of FIG. 6B (30′c(ii)) was then eluted with 0.1M NaOH, run on an Invitrogen precast 20% UREA-TBE gel and stained with SybrGold. A second sample (Run G) was prepared and sequenced using the same method and equipment as Run F, with the addition of nick translation performed by injection of Pol(X) coupled to GAN to incorporate and nick simultaneously. The nicked portions of DNA strand 30′c(ii) (FIG. 6B) were eluted, run on a 20% UREA-TBE gel and stained with SybrGold.

A third sample (Run H) was prepared and sequenced using the same method and equipment as Run F, with the addition of nick translation performed by addition of Sso7d coupled to GAN to incorporation mix to incorporate and nick simultaneously. The nicked portions of DNA strand 30′c(ii) (FIG. 6B) were eluted, run on a 20% UREA-TBE gel and stained with SybrGold.

Sso7d was encoded by the DNA sequence ATGGCAACCGTCAAGTTTAAATACAAAGGCGAGGAGAAGGAGGTGGACATC AGCAAAATCAAAAAAGTATGGCGTGTCGGGAAAATGATTTCGTTTACCTACG ACGAGGGCGGGGGGAAGACCGGACGTGGAGCAGTATCAGAAAAGGATGCCC CGAAAGAACTTTTGCAAATGCTTGAAAAACAGAAAAAG (SEQ ID NO: 9) and was attached to GAN with the linker sequence GCGGCATTAGGTGGTGCAGCAGCCGCGGCAGCGTCG (SEQ ID NO: 10).

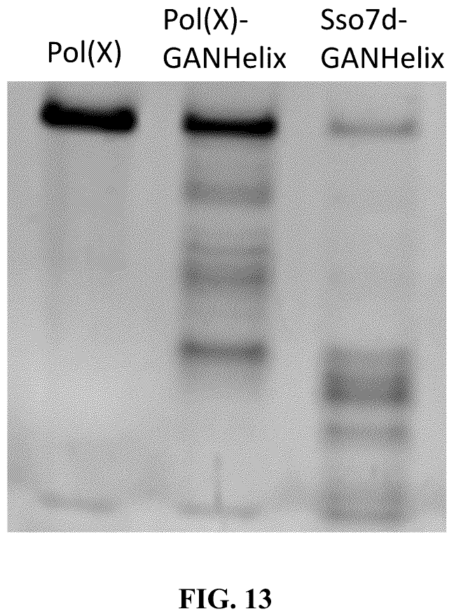

The results of the UREA-TBE gel electrophoresis of the digested flaps from Runs F, G, and H (FIG. 13) show that the flap fragments digested after injection of Sso7d coupled to GAN (Run H) were smaller than those digested after injection of Pol(X) coupled to GAN (Run G). The largest flap fragments were from Run F, which did not include a flap nuclease.

### Example 4

Illustration of a double-stranded sequencing technique commensurate with one or more embodiments of sequencing method 300 of the present disclosure.

Sequencing performance of sequencing techniques commensurate with embodiments of method 300 were compared to a standard single stranded sequencing (control sample Run I) using an Illumina NextSeq2000 sequencing by synthesis system. All samples produced and sequenced used a DNA library containing a portion of the human genome.

A second sample (Run J) was prepared and sequenced using a technique commensurate with embodiments of method 300 of the present disclosure. In this sample, DNA clusters were produced using the standard NextSeq2000 protocol, replacing the Read 1 primer with a primer containing four uracils. The primer was elongated to form a bridged double stranded oligonucleotide complex to produce clusters of double stranded oligonucleotide complexes as shown in the first step of FIG. 8B (PSC2). The uracil specific excision reagent (USER; available from New England BioLabs, Ipswich, MA) was incubated with the clusters to cleave the uracils from the primer (e.g., Step B of FIG. 8B) to produce multiple fragments (e.g., step B in FIG. 8B) sufficiently small that they dissociate at the temperature used (60° C.). The standard Read 1 primer (85) was then hybridized to the DNA strand (30). Ssequencing by synthesis was carried out, starting from the hybridized Read 1 primer, as described in U.S. Pat. No. 11,293,061B2 and US Patent Pub. No. US 2021/0403500A1. 151 cycles of SBS were performed using 3 μM Pol(X). Nick translation was performed by injection of Pol(X) coupled to GAN to incorporate and nick simultaneously. A 1 M NaCl salt wash was performed after each incorporation step.

A third sample (Run K) was prepared and sequenced using the same method and equipment as Run J, with the exception that nick translation was performed by injection of a mixture of Pol(X) and Sso7d coupled to GAN (instead of Pol(X) coupled to GAN) to incorporate and nick simultaneously.

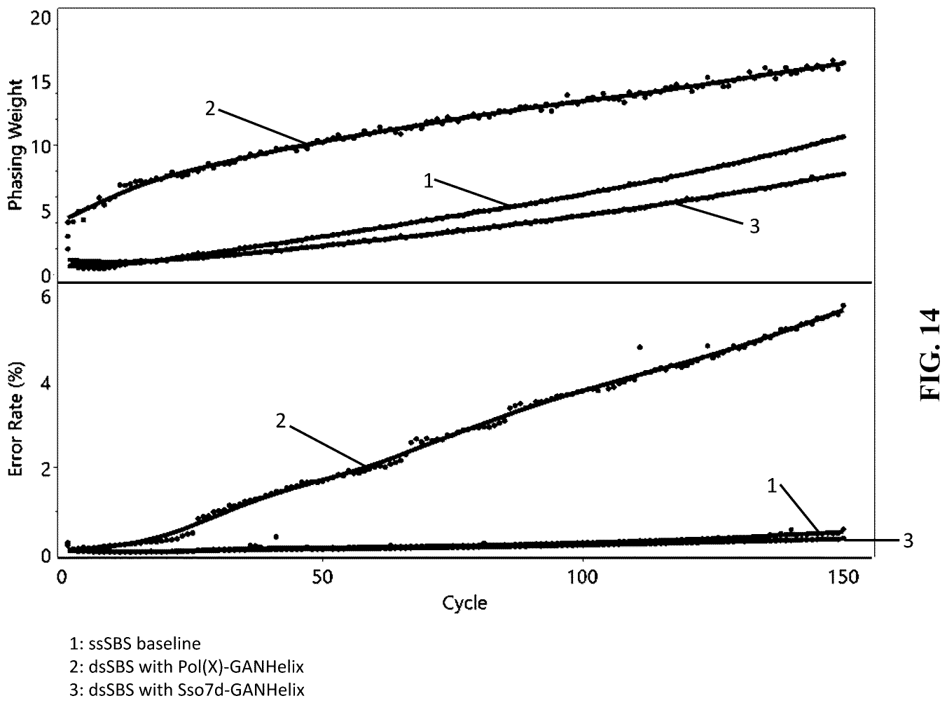

The results of FIG. 14 show the improved sequencing metrics for nick translation in Run K (injection of Sso7d coupled to GAN) compared to Run J (injection of Pol(X) coupled to GAN), as evidenced by the decreased error rate.

A number of embodiments have been described. Nevertheless, it will be understood that various modifications may be made. Accordingly, other embodiments are within the scope of the following claims.

SEQUENCE LISTING FREE TEXT

SEQ ID NO. 1: P5
TTTTTTTTTT AATGATACGG CGACCACCGA GANCTACAC
where N is uracil.
SEQ ID NO. 2: P7
TTTTTTTTTT CAAGCAGAAG ACGGCATACG ANAT
where N is 8-oxo-guanine.
SEQ ID NO. 3: P15
TTTTTTAATG ATACGGCGAC CACCGAGANC TACAC
where N is allyl T nucleoside.
SEQ ID NO. 4: P17
TTTTTINNNC AAGCAGAAGA CGGCATACGA GAT
where N is

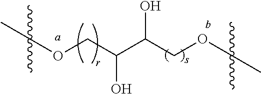

or

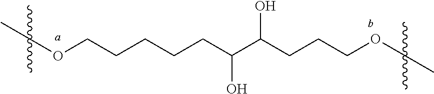

where r is 2, 3, 4, 5, or 6;
s is 2, 3, 4, 5, or 6;
the “a” oxygen is the 3 hydroxyl oxygen of a first
nucleotide and the “b” is the 5′ hydroxyl
oxygen of a second nucleotide.
SEQ ID NO. 5: Peptide Linker 1
GGGGSGGGGSGGGGS
SEQ ID NO. 6: Peptide Linker 2
AALGGAAAAAAS
SEQ ID NO. 7: Peptide Linker 3
ALEEAPWPPPWGA
SEQ ID NO. 8: DNA monotemplate
AATGATACGGCGACCACCGAGATCTACACCTAGGAGCTAAAGCGAGATCGG
AAGAGCGCGTGTAGGGAAAGAGTGTATCTCGTATGCCGTCTTCTGCTTG
SEQ ID NO. 9: Sso7d DNA sequence
ATGGCAACCGTCAAGTTTAAATACAAAGGCGAGGAGAAGGAGGTGGACATC
AGCAAAATCAAAAAAGTATGGCGTGTCGGGAAAATGATTTCGTTTACCTAC
GACGAGGGCGGGGGGAAGACCGGACGTGGAGCAGTATCAGAAAAGGATGCC
CCGAAAGAACTTTTGCAAATGCTTGAAAAACAGAAAAAG
SEQ ID NO. 10: Peptide Linker 4
GCGGCATTAGGTGGTGCAGCAGCCGCGGCAGCGTCG

1.-44. (canceled)

45. A nucleotide sequencing method comprising:
(a) providing a surface-bound double-stranded oligonucleotide complex comprising a first oligonucleotide strand, a second oligonucleotide strand hybridized to the first nucleotide strand, and a primer hybridized to the first oligonucleotide strand, wherein the first oligonucleotide strand has a 5′ end bound to a surface, and wherein a free 3′ end of the primer is hybridized to a nucleotide of the first oligonucleotide strand that is 3′ of a nucleotide of the first oligonucleotide strand to which a 5′ end of the second oligonucleotide strand is hybridized;
(b) extending the primer from the free 3′ end using the first oligonucleotide strand as a template and sequencing at least a portion of the first oligonucleotide strand via sequencing by synthesis as the primer is extended; and
(c) nicking the second strand to remove a 5′ portion of the second strand before, during, or after one or more nucleotides are added to extend the primer in step (b).

46. The method of claim 45, wherein the second strand is nicked in step (c) before, during, or after each nucleotide is added to extend the primer in step (b).

47. The method of claim 45, wherein the second strand is nicked in step (c) after more than one nucleotide is added to extend the primer in step (b).

48. The method of claim 45, wherein nicking the second strand to remove a 5′ portion of the second strand comprises removing nucleotides and/or oligonucleotides from the second strand using a flap nuclease.

49. The method of claim 48, wherein the flap nuclease comprises a GINS-associated nuclease (GAN), a Taq DNA polymerase, a Bst DNA polymerase, or FEN1.

50. The method of claim 48, wherein the flap nuclease is operably linked to a second protein.

51. The method of claim 50, wherein the second protein comprises a polymerase or a dsDNA binding domain.

52. The method of claim 51, wherein the dsDNA binding domain comprises a sequence-nonspecific DNA binding domain.

53. The method of claim 52, wherein the DNA binding domain comprises Sso7d.

54. A nucleotide sequencing method comprising:
providing a first surface-bound double-stranded oligonucleotide complex comprising a first oligonucleotide strand and a second oligonucleotide strand, wherein the first oligonucleotide strand has a 5′ end bound to a surface;
exposing the first surface-bound double-stranded oligonucleotide complex to a nuclease to cleave the second oligonucleotide strand and produce a cleaved first portion and a cleaved second portion of the second oligonucleotide strand, wherein the cleaved first and second portions are hybridized to the first oligonucleotide strand, and wherein the cleaved first portion comprises a free 3′ end; and
extending the cleaved first portion from the free 3′ end using the first oligonucleotide strand as a template and sequencing at least a portion of the first oligonucleotide strand as the cleaved first portion is extended.

55. The nucleotide sequencing method of claim 54, wherein the cleaved first portion of the second oligonucleotide strand has a 5′ end bound to the surface.

56. The nucleotide sequencing method of claim 54, wherein extending the surface bound cleaved first portion generates a second surface-bound double-stranded oligonucleotide complex comprising the first oligonucleotide strand and a nascent third oligonucleotide strand comprising the surface bound cleaved first portion.

57. The method of claim 54, wherein a ribonucleoprotein comprises a guide RNA (gRNA) and the nuclease, and wherein the gRNA hybridizes to a portion of the first oligonucleotide strand or a portion of the second oligonucleotide strand.

58. The method of claim 54, wherein extending the cleaved first portion from the free 3′ end results in the displacement of at least a 5′ portion of the cleaved second portion from the first oligonucleotide strand.

59. The method of claim 54, wherein extending the cleaved first portion from the free 3′ end further comprises:
removing nucleotides and/or oligonucleotides from the cleaved second portion using a flap nuclease.

60. The nucleotide sequencing of claim 54, further comprising:
exposing the second surface-bound double-stranded oligonucleotide complex to a second nuclease to cleave the first oligonucleotide strand and produce cleaved third and cleaved fourth cleaved portions of the first oligonucleotide strand, wherein the cleaved third and cleaved fourth portions are hybridized to the third oligonucleotide strand, and wherein the cleaved third portion comprises a surface bound 5′ end and a free 3′ end; and
extending the cleaved third portion from the free 3′ end using the third oligonucleotide strand as a template and sequencing at least a portion of the third oligonucleotide strand as the cleaved third portion is extended.

61. A nucleotide sequencing method comprising:
providing a first oligonucleotide strand having a 5′ end bound to a surface;
hybridizing an extension primer to a portion of the first oligonucleotide strand, the extension primer having a free 3′ end and a cleavage site;
extending the extension primer from the free 3′ end using the first oligonucleotide strand as a template to produce a second oligonucleotide strand hybridized to the surface-bound first oligonucleotide strand, the second oligonucleotide strand comprising the extension primer;
cleaving the extension primer of the second oligonucleotide strand at the cleavage site to produce cleaved first and second portions of the second oligonucleotide strand, wherein the cleaved first and second portions are hybridized to the first oligonucleotide strand, and wherein the cleaved first portion comprises a free 3′ end; and
extending the cleaved first portion from the free 3′ end using the first oligonucleotide strand as a template and sequencing at least a portion of the first oligonucleotide strand as the cleaved first portion is extended.

62. The nucleotide sequencing method of claim 61, wherein the first oligonucleotide strand comprises a 3′ portion, and wherein the method further comprises providing a surface oligonucleotide having a free 3′ end and having a 5′ end bound to the surface, wherein the 3′ portion of the first oligonucleotide is hybridized to at least a portion of the surface oligonucleotide.

63. The method of claim 61, wherein cleaving the extension primer of the second oligonucleotide strand at the cleavage site further comprises:
removing a first excisable base from the primer to produce the cleaved first portion; or
treating the extension primer of the second oligonucleotide strand with one or more dihydroxylation reagents to produce the cleaved first portion.

64. The method of claim 61, wherein extending the cleaved first portion from the free 3′ end further comprises:
removing nucleotides and/or oligonucleotides from the cleaved second portion using a flap nuclease.


SEQUENCE LISTING (10 sequences)

SEQ ID NO: 1 - DNA, 39 bp, synthetic construct
  source 1..39: mol_type = other DNA; note = N is uracil
ttttttttttaatgatacggcgaccaccgaganctacac

SEQ ID NO: 2 - DNA, 34 bp, synthetic construct
  source 1..34: mol_type = other DNA; note = n is 8-oxo-guanine
ttttttttttcaagcagaagacggcatacganat

SEQ ID NO: 3 - DNA, 35 bp, synthetic construct
  source 1..35: mol_type = other DNA; note = n is allyl T nucleoside
ttttttaatgatacggcgaccaccgaganctacac

SEQ ID NO: 4 - DNA, 24 bp, synthetic construct
  source 1..24: mol_type = other DNA
caagcagaagacggcatacgagat

SEQ ID NO: 5 - protein, 15 aa, synthetic construct
GGGGSGGGGSGGGGS

SEQ ID NO: 6 - protein, 12 aa, synthetic construct
AALGGAAAAAAS

SEQ ID NO: 7 - protein, 13 aa, synthetic construct
ALEEAPWPPPWGA

SEQ ID NO: 8 - DNA, 101 bp, synthetic construct
  source 1..101: mol_type = other DNA
aatgatacggcgaccaccgagatctacacctaggagctaaagcgagatcggaagagcgtcgtgtagggaaagagtgtatctcgtatgccgtcttctgcttg

SEQ ID NO: 9 - DNA, 192 bp, synthetic construct
  source 1..192: mol_type = other DNA
atggcaaccgtcaagtttaaatacaaaggcgaggagaaggaggtggacatcagcaaaatcaaaaaagtatggcgtgtcgggaaaatgatttcgtttacctacgacgagggcggggggaagaccggacgtggagcagtatcagaaaaggatgccccgaaagaacttttgcaaatgcttgaaaaacagaaaaag

SEQ ID NO: 10 - protein, 36 aa, synthetic construct
GCGGCATTAGGTGGTGCAGCAGCCGCGGCAGCGTCG